SEQUENCE LISTING (4 sequences)

SEQ ID NO: 1 - DNA, 2433 bp, Homo sapiens
atgattccggtgacagagctccgctactttgcggacacgcagccagcataccggatcctgaagccgtggtgggatgtgttcacagactacatctctatcgtcatgctgatgattgccgtcttcggggggacgctgcaggtcacccaagacaagatgatctgcctgccttgtaagtgggtcaccaaggactcctgcaatgattcgttccggggctgggcagcccctggcccggagcccacctaccccaactccaccattctgccgacccctgacacgggccccacaggcatcaagtatgacctggaccggcaccagtacaactacgtggacgctgtgtgctatgagaaccgactgcactggtttgccaagtacttcccctacctggtgcttctgcacacgctcatcttcctggcctgcagcaacttctggttcaaattcccgcgcaccagctcgaagctggagcactttgtgtctatcctgctgaagtgcttcgactcgccctggaccacgagggccctgtcggagacagtggtggaggagagcgaccccaagccggccttcagcaagatgaatgggtccatggacaaaaagtcatcgaccgtcagtgaggacgtggaggccaccgtgcccatgctgcagcggaccaagtcacggatcgagcagggtatcgtggaccgctcagagacgggcgtgctggacaagaaggagggggagcaagccaaggcgctgtttgagaaggtgaagaagttccggacccatgtggaggagggggacattgtgtaccgcctctacatgcggcagaccatcatcaaggtgatcaagttcatcctcatcatctgctacaccgtctactacgtgcacaacatcaagttcgacgtggactgcaccgtggacattgagagcctgacgggctaccgcacctaccgctgtgcccaccccctggccacactcttcaagatcctggcgtccttctacatcagcctagtcatcttctacggcctcatctgcatgtatacactgtggtggatgctacggcgctccctcaagaagtactcgtttgagtcgatccgtgaggagagcagctacagcgacatccccgacgtcaagaacgacttcgccttcatgctgcacctcattgaccaatacgacccgctctactccaagcgcttcgccgtcttcctgtcggaggtgagtgagaacaagctgcggcagctgaacctcaacaacgagtggacgctggacaagctccggcagcggctcaccaagaacgcgcaggacaagctggagctgcacctgttcatgctcagtggcatccctgacactgtgtttgacctggtggagctggaggtcctcaagctggagctgatccccgacgtgaccatcccgcccagcattgcccagctcacgggcctcaaggagctgtggctctaccacacagcggccaagattgaagcgcccgcgctggccttcctgcgcgagaacctgcgggcgctgcacatcaagttcaccgacatcaaggagatcccgctgtggatctatagcctgaagacactggaggagctgcacctgacgggcaacctgagcgcggagaacaaccgctacatcgtcatcgacgggctgcgggagctcaaacgcctcaaggtgctgcggctcaagagcaacctaagcaagctgccacaggtggtcacagatgtgggcgtgcacctgcagaagctgtccatcaacaatgagggcaccaagctcatcgtcctcaacagcctcaagaagatggcgaacctgactgagctggagctgatccgctgtgacctggagcgcatcccccactccatcttcagcctccacaacctgcaggagattgacctcaaggacaacaacctcaagaccatcgaggagatcatcagcttccagcacctgcaccgcctcacctgccttaagctgtggtacaaccacatcgcctacatccccatccagatcggcaacctcaccaacctggagcgcctctacctgaaccgcaacaagatcgagaagatccccacccagctcttctactgccgcaagctgcgctacctggacctcagccacaacaacctgaccttcctccctgccgacatcggcctcctgcagaacctccagaacctagccatcacggccaaccggatcgagacgctccctccggagctcttccagtgccggaagctgcgggccctgcacctgggcaacaacgtgctgcagtcactgccctccagggtgggcgagctgaccaacctgacgcagatcgagctgcggggcaaccggctggagtgcctgcctgtggagctgggcgagtgcccactgctcaagcgcagcggcttggtggtggaggaggacctgttcaacacactgccacccgaggtgaaggagcggctgtggagggctgacaaggagcaggcctga

SEQ ID NO: 2 - protein, 810 aa, Homo sapiens
MIPVTELRYFADTQPAYRILKPWWDVFTDYISIVMLMIAVFGGTLQVTQDKMICLPCKWVTKDSCNDSFRGWAAPGPEPTYPNSTILPTPDTGPTGIKYDLDRHQYNYVDAVCYENRLHWFAKYFPYLVLLHTLIFLACSNFWFKFPRTSSKLEHFVSILLKCFDSPWTTRALSETVVEESDPKPAFSKMNGSMDKKSSTVSEDVEATVPMLQRTKSRIEQGIVDRSETGVLDKKEGEQAKALFEKVKKFRTHVEEGDIVYRLYMRQTIIKVIKFILIICYTVYYVHNIKFDVDCTVDIESLTGYRTYRCAHPLATLFKILASFYISLVIFYGLICMYTLWWMLRRSLKKYSFESIREESSYSDIPDVKNDFAFMLHLIDQYDPLYSKRFAVFLSEVSENKLRQLNLNNEWTLDKLRQRLTKNAQDKLELHLFMLSGIPDTVFDLVELEVLKLELIPDVTIPPSIAQLTGLKELWLYHTAAKIEAPALAFLRENLRALHIKFTDIKEIPLWIYSLKTLEELHLTGNLSAENNRYIVIDGLRELKRLKVLRLKSNLSKLPQVVTDVGVHLQKLSINNEGTKLIVLNSLKKMANLTELELIRCDLERIPHSIFSLHNLQEIDLKDNNLKTIEEIISFQHLHRLTCLKLWYNHIAYIPIQIGNLTNLERLYLNRNKIEKIPTQLFYCRKLRYLDLSHNNLTFLPADIGLLQNLQNLAITANRIETLPPELFQCRKLRALHLGNNVLQSLPSRVGELTNLTQIELRGNRLECLPVELGECPLLKRSGLVVEEDLFNTLPPEVKERLWRADKEQA

SEQ ID NO: 3 - protein, 15 aa, Homo sapiens
CEVKERLWRADKEQA

SEQ ID NO: 4 - protein, 18 aa, Homo sapiens
LPTPDTGPTGIKYDLDRH

(19) United States
(12) Patent Application Publication
(10) Pub. No.: US 2021/0324057 A1
(43) Pub. Date: Oct. 21, 2021

(54) METHODS AND COMPOSITIONS FOR TREATING AND PREVENTING T CELL-DRIVEN DISEASES

(71) Applicant: THE CHILDREN'S MEDICAL CENTER CORPORATION, Boston, MA (US)

(72) Inventors: Janet S. CHOU, Cambridge, MA (US); Raif S. GEHA, Belmont, MA (US)

(73) Assignee: THE CHILDREN'S MEDICAL CENTER CORPORATION, Boston, MA (US)

(21) Appl. No.: 17/288,600

(22) PCT Filed: Oct. 25, 2019
(86) PCT No.: PCT/US2019/058114

§ 371 Date: Apr. 26, 2021

Related U.S. Application Data
(60) Provisional application No. 62/752,738, filed on Oct. 30, 2018.
(60) Provisional application No. 62/775,099, filed on Dec. 4, 2018.

(51) Int. Cl. C07K 16/18; A61K 38/28; A61K 45/06; C12N 15/113; C07K 14/705

(52) U.S. Cl. CPC C07K 16/18; A61K 38/28; A61K 45/06; C12N 15/113; C07K 2319/30; C12N 2320/31; C12N 2310/141; C12N 2310/531; C07K 14/70521

(57) ABSTRACT

Described herein are methods and compositions for treating graft versus host disease. Additionally, described herein are methods and compositions for treating diabetes. Aspects of the invention relates to administering to a subject an agent that inhibits LRRC8A as a monotherapy or in combination with additional therapeutics.

### CROSS REFERENCE TO RELATED APPLICATIONS

This application is a 371 National Phase Entry of International Patent Application No. PCT/US2019/058114 filed on Oct. 25, 2019, which claims benefit under 35 U.S.C. § 119(e) of U.S. Provisional Application Nos. 62/752,738 filed on Oct. 30, 2018 and 62/775,099 filed on Dec. 4, 2018, the contents of which are incorporated herein by reference in their entirety.

### GOVERNMENT SUPPORT

This invention was made with Government support under Grant No: 1K08A1116979-01 awarded by the National Institutes of Health. The Government has certain rights in the invention.

### FIELD OF INVENTION

The field of the invention relates to the treatment and prevention of T cell activation and T cell-driven diseases, namely graft versus host disease and diabetes.

### SEQUENCE LISTING

The instant application contains a Sequence Listing which has been submitted electronically in ASCII format and is hereby incorporated by reference in its entirety. Said ASCII copy, created on Apr. 20, 2021, is named 701039-093810WOPT_SL.txt and is 11,013 bytes in size.

### BACKGROUND

T cell-driven organ damage remains a challenge in the treatment of primary immunodeficiencies with T cell dysregulation, in acute graft versus host disease after hematopoietic stem cell transplantation, and in diabetes. The variable clinical response to existing immunosuppressive therapies indicates a need for defining and targeting additional mechanisms of T cell activation.

### SUMMARY

The present invention is based, in part, on the premise that costimulatory molecules enhance T cell activation by modulating TCR-driven signaling and cellular metabolism. Data presented herein indicate that LRRC8A co-localizes with the TCR and promotes a transcriptional signature characteristic of T cell activation. in vitro and in vivo data presented herein show that conditional deficiency of LRRC8A impairs the effector and metabolic functions of antigen-experienced CD4⁺ and CD8⁺ T cells. Conditional deletion of LRRC8A on donor CD4⁺ T cells attenuates acute GVHD, demonstrating the relevance of LRRC8A in T cell-driven diseases.

Accordingly, provided herein is a method for treating or preventing graft versus host disease, the method comprising administering to a subject having, or at risk of developing, graft versus host disease an agent that inhibits LRRC8A.

Another aspect provided herein is a method for treating or preventing graft versus host disease, the method comprising administering to a subject in need thereof an agent that inhibits LRRC8A, wherein the agent is an antibody binds an antigen having a sequence selected from the group consisting of: SEQ ID NO: 3 and SEQ ID NO: 4.

In one embodiment of any aspect, the method further comprises, prior to administering, the step of diagnosing a subject as having, or at risk of developing, graft versus host disease.

In one embodiment of any aspect, the method further comprises, prior to administering, the step of receiving the results from an assay that identifies a subject as having, or at risk of developing, graft versus host disease.

In one embodiment of any aspect, the subject is an organ transplant or hematopoietic stem cell transplant recipient.

In one embodiment of any aspect, LRRC8A is inhibited in a T cell or antigen presenting cell (APC e.g., a macrophage, a dendritic cell, a B-cell, an artificial APC, and the like).

In one embodiment of any aspect, inhibiting results in the blocking of an extracellular portion of LRRC8A on a T cell or antigen presenting cell.

In one embodiment of any aspect, the agent that inhibits LRRC8A is selected from the group consisting of a small molecule, an antibody, a peptide, a genome editing system, an antisense oligonucleotide, and an RNAi. In one embodiment of any aspect, the RNAi is a microRNA, an siRNA, or a shRNA.

In one embodiment of any aspect, the antibody targets an antigen having a sequence selected from the group consisting of: SEQ ID NO: 3 and SEQ ID NO: 4.

In one embodiment of any aspect, wherein inhibiting LRRC8A is inhibiting the expression level and/or activity of LRRC8A. In one embodiment of any aspect, the expression level and/or activity of LRRC8A is inhibited by at least 50%, at least 60%, at least 70%, at least 80%, at least 90%, or more as compared to an appropriate control.

In one embodiment of any aspect, the method further comprises administering at least a second therapeutic. In one embodiment of any aspect, the second therapeutic is Abatacept (Orencia®) or Belatacept (Nulojix®).

Another aspect provided herein is a method of treating diabetes comprising administering to a subject in need thereof an agent that inhibits LRRC8A. In one embodiment, diabetes is type 1 diabetes or type 2 diabetes.

Yet another aspect provided herein is a method for treating diabetes, the method comprising administering to a subject in need thereof an agent that inhibits LRRC8A, wherein the agent is an antibody binds an antigen having a sequence selected from the group consisting of: SEQ ID NO: 3 and SEQ ID NO: 4.

In one embodiment of any aspect, the method further comprises, prior to administering, the step of diagnosing a subject as having diabetes.

In one embodiment of any aspect, the method further comprises, prior to administering, the step of receiving the results from an assay that identifies a subject as having diabetes.

In one embodiment of any aspect, the method further comprises administering at least a second therapeutic. In one embodiment of any aspect, the second therapeutic is insulin, Abatacept (Orencia®), or Belatacept (Nulojix®).

Another aspect provided herein is a composition comprising an agent that inhibits LRRC8A, wherein the agent is an antibody binds an antigen having a sequence selected from the group consisting of: SEQ ID NO: 3 and SEQ ID NO: 4.

In one embodiment of any aspect, the composition further comprises at least a second therapeutic. In one embodiment of any aspect, the second therapeutic is selected from the group consisting of: insulin, Abatacept (Orencia®) or Belatacept (Nulojix®).

In one embodiment of any aspect, the composition further comprises a pharmaceutically acceptable carrier or diluent.

Another aspect provided herein is the use of any of the compositions described herein for the treatment of graft versus host disease.

Another aspect provided herein is the use of any of the compositions described herein for the treatment of diabetes.

### Definitions

For convenience, the meaning of some terms and phrases used in the specification, examples, and appended claims, are provided below. Unless stated otherwise, or implicit from context, the following terms and phrases include the meanings provided below. The definitions are provided to aid in describing particular embodiments, and are not intended to limit the claimed technology, because the scope of the technology is limited only by the claims. Unless otherwise defined, all technical and scientific terms used herein have the same meaning as commonly understood by one of ordinary skill in the art to which this technology belongs. If there is an apparent discrepancy between the usage of a term in the art and its definition provided herein, the definition provided within the specification shall prevail.

As used herein, the terms “treat,” “treatment,” “treating,” or “amelioration” refer to therapeutic treatments, wherein the object is to reverse, alleviate, ameliorate, inhibit, slow down or stop the progression or severity of a condition associated with an autoimmune disease, for example GVHD or diabetes. The term “treating” includes reducing or alleviating at least one adverse effect or symptom of an autoimmune disease (e.g., GVHD or diabetes). Treatment is generally “effective” if one or more symptoms or clinical markers are reduced. Alternatively, treatment is “effective” if the progression of a disease is reduced or halted. That is, “treatment” includes not just the improvement of symptoms or markers, but also a cessation of, or at least slowing of, progress or worsening of symptoms compared to what would be expected in the absence of treatment. Beneficial or desired clinical results include, but are not limited to, alleviation of one or more symptom(s), diminishment of extent of disease, stabilized (i.e., not worsening) state of disease, delay or slowing of disease progression, amelioration or palliation of the disease state, remission (whether partial or total), and/or decreased mortality, whether detectable or undetectable. The term “treatment” of a disease also includes providing relief from the symptoms or side-effects of the disease (including palliative treatment).

As used herein “preventing” or “prevention” refers to any methodology where the disease state or disorder (e.g., GVHD or diabetes) does not occur due to the actions of the methodology (such as, for example, administration of an agent that inhibits LRRC8A, or a composition described herein). In one aspect, it is understood that prevention can also mean that the disease is not established to the extent that occurs in untreated controls. For example, there can be a 5, 10, 15, 20, 25, 30, 35, 40, 50, 60, 70, 80, 90, or 100% reduction in the establishment of disease frequency relative to untreated controls. Accordingly, prevention of a disease encompasses a reduction in the likelihood that a subject will develop the disease, relative to an untreated subject (e.g. a subject who is not treated with a composition comprising a microbial consortium as described herein).

As used herein, the term “administering,” refers to the placement of a therapeutic (e.g., an agent that inhibits LRRC8A) or pharmaceutical composition as disclosed herein into a subject by a method or route which results in at least partial delivery of the agent to the subject. Pharmaceutical compositions comprising agents as disclosed herein can be administered by any appropriate route which results in an effective treatment in the subject.

As used herein, a “subject” means a human or animal. Usually the animal is a vertebrate such as a primate, rodent, domestic animal or game animal. Primates include, for example, chimpanzees, cynomolgus monkeys, spider monkeys, and macaques, e.g., Rhesus. Rodents include, for example, mice, rats, woodchucks, ferrets, rabbits and hamsters. Domestic and game animals include, for example, cows, horses, pigs, deer, bison, buffalo, feline species, e.g., domestic cat, canine species, e.g., dog, fox, wolf, avian species, e.g., chicken, emu, ostrich, and fish, e.g., trout, catfish and salmon. In some embodiments, the subject is a mammal, e.g., a primate, e.g., a human. The terms, “individual,” “patient” and “subject” are used interchangeably herein.

Preferably, the subject is a mammal. The mammal can be a human, non-human primate, mouse, rat, dog, cat, horse, or cow, but is not limited to these examples. Mammals other than humans can be advantageously used as subjects that represent animal models of disease e.g., GVHD or diabetes. A subject can be male or female. A subject can be a child (e.g., less than 18 years of age), or an adult (e.g., greater than 18 years of age).

A subject can be one who has been previously diagnosed with or identified as suffering from or having a T cell-driven disease or disorder in need of treatment (e.g., GVHD or diabetes) or one or more complications related to such a disease or disorder, and optionally, have already undergone treatment for the disease or disorder or the one or more complications related to the disease or disorder. Alternatively, a subject can also be one who has not been previously diagnosed as having such disease or disorder (e.g., GVHD or diabetes) or related complications. For example, a subject can be one who exhibits one or more risk factors for the disease or disorder or one or more complications related to the disease or disorder or a subject who does not exhibit risk factors.

As used herein, an “agent” refers to e.g., a molecule, protein, peptide, antibody, or nucleic acid, that inhibits expression of a polypeptide or polynucleotide, or binds to, partially or totally blocks stimulation, decreases, prevents, delays activation, inactivates, desensitizes, or down regulates the activity of the polypeptide or the polynucleotide. Agents that inhibit LRRC8A, e.g., inhibit expression, e.g., translation, post-translational processing, stability, degradation, or nuclear or cytoplasmic localization of a polypeptide, or transcription, post transcriptional processing, stability or degradation of a polynucleotide or bind to, partially or totally block stimulation, DNA binding, transcription factor activity or enzymatic activity, decrease, prevent, delay activation, inactivate, desensitize, or down regulate the activity of a polypeptide or polynucleotide. An agent can act directly or indirectly.

The term “agent” as used herein means any compound or substance such as, but not limited to, a small molecule, nucleic acid, polypeptide, peptide, drug, ion, etc. An “agent” can be any chemical, entity or moiety, including without limitation synthetic and naturally-occurring proteinaceous and non-proteinaceous entities. In some embodiments, an agent is nucleic acid, nucleic acid analogues, proteins, antibodies, peptides, aptamers, oligomer of nucleic acids, amino acids, or carbohydrates including without limitation proteins, oligonucleotides, ribozymes, DNAzymes, glycoproteins, siRNAs, lipoproteins, aptamers, and modifications and combinations thereof etc. In certain embodiments, agents are small molecule having a chemical moiety. For example, chemical moieties included unsubstituted or substituted alkyl, aromatic, or heterocyclyl moieties including macrolides, leptomycins and related natural products or analogues thereof. Compounds can be known to have a desired activity and/or property, or can be selected from a library of diverse compounds.

The agent can be a molecule from one or more chemical classes, e.g., organic molecules, which may include organometallic molecules, inorganic molecules, genetic sequences, etc. Agents may also be fusion proteins from one or more proteins, chimeric proteins (for example domain switching or homologous recombination of functionally significant regions of related or different molecules), synthetic proteins or other protein variations including substitutions, deletions, insertion and other variants.

As used herein, the term “small molecule” refers to a chemical agent which can include, but is not limited to, a peptide, a peptidomimetic, an amino acid, an amino acid analog, a polynucleotide, a polynucleotide analog, an aptamer, a nucleotide, a nucleotide analog, an organic or inorganic compound (e.g., including heterorganic and organometallic compounds) having a molecular weight less than about 10,000 grams per mole, organic or inorganic compounds having a molecular weight less than about 5,000 grams per mole, organic or inorganic compounds having a molecular weight less than about 1,000 grams per mole, organic or inorganic compounds having a molecular weight less than about 500 grams per mole, and salts, esters, and other pharmaceutically acceptable forms of such compounds.

The term “RNAi” as used herein refers to interfering RNA or RNA interference. RNAi refers to a means of selective post-transcriptional gene silencing by destruction of specific mRNA by molecules that bind and inhibit the processing of mRNA, for example inhibit mRNA translation or result in mRNA degradation. As used herein, the term “RNAi” refers to any type of interfering RNA, including but are not limited to, siRNA, shRNA, endogenous microRNA and artificial microRNA. For instance, it includes sequences previously identified as siRNA, regardless of the mechanism of down-stream processing of the RNA (i.e. although siRNAs are believed to have a specific method of in vivo processing resulting in the cleavage of mRNA, such sequences can be incorporated into the vectors in the context of the flanking sequences described herein).

Methods and compositions described herein require that the levels and/or activity of LRRC8A are inhibited. As used herein, “Lucine rich repeat containing 8 VRAC subunit A” or “LRRC8A”, also known as AGMS, LRRC8, and SWELL1, refers to a gene involved in diverse biological processes, including cell adhesion, cellular trafficking, and hormone-receptor interactions. This family member is a putative four-pass transmembrane protein that plays a role in B cell development. LRRC8A sequences are known for a number of species, e.g., human LRRC8A (NCBI Gene ID: 56262) polypeptide (e.g., NCBI Ref Seq NP_001120716.1) and mRNA (e.g., NCBI Ref Seq NM_001127244.1). LRRC8A can refer to human LRRC8A, including naturally occurring variants, molecules, and alleles thereof. LRRC8A refers to the mammalian LRRC8A of, e.g., mouse, rat, rabbit, dog, cat, cow, horse, pig, and the like. The nucleic sequence of SEQ ID NO: 1 comprises a nucleic sequence which encodes LRRC8A.

The term “decrease”, “reduced”, “reduction”, or “inhibit” are all used herein to mean a decrease by a statistically significant amount. In some embodiments, “decrease”, “reduced”, “reduction”, or “inhibit” typically means a decrease by at least 10% as compared to an appropriate control (e.g. the absence of a given treatment) and can include, for example, a decrease by at least about 10%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, at least about 99%, or more. As used herein, “reduction” or “inhibition” does not encompass a complete inhibition or reduction as compared to a reference level. “Complete inhibition” is a 100% inhibition as compared to an appropriate control.

The terms “increase”, “enhance”, or “activate” are all used herein to mean an increase by a reproducible statistically significant amount. In some embodiments, the terms “increase”, “enhance”, or “activate” can mean an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, a 20 fold increase, a 30 fold increase, a 40 fold increase, a 50 fold increase, a 6 fold increase, a 75 fold increase, a 100 fold increase, etc. or any increase between 2-fold and 10-fold or greater as compared to an appropriate control. In the context of a marker, an “increase” is a reproducible statistically significant increase in such level.

As used herein, a “reference level” refers to a normal, otherwise unaffected cell population or tissue (e.g., a biological sample obtained from a healthy subject, or a biological sample obtained from the subject at a prior time point, e.g., a biological sample obtained from a patient prior to being diagnosed with an autoimmune disease (e.g., GVHD or diabetes) or a biological sample that has not been contacted with an agent disclosed herein).

As used herein, an “appropriate control” refers to an untreated, otherwise identical cell or population (e.g., a patient who was not administered an agent, e.g., an agent that inhibits LRRC8, described herein, or was administered by only a subset of agents described herein, as compared to a non-control cell).

The term “statistically significant” or “significantly” refers to statistical significance and generally means a two standard deviation (2SD) or greater difference.

As used herein the term “comprising” or “comprises” is used in reference to compositions, methods, and respective component(s) thereof, that are essential to the method or composition, yet open to the inclusion of unspecified elements, whether essential or not.

The singular terms “a,” “an,” and “the” include plural referents unless context clearly indicates otherwise. Similarly, the word “or” is intended to include “and” unless the context clearly indicates otherwise. Although methods and materials similar or equivalent to those described herein can be used in the practice or testing of this disclosure, suitable methods and materials are described below. The abbreviation, “e.g.” is derived from the Latin exempli gratia, and is used herein to indicate a non-limiting example. Thus, the abbreviation “e.g.” is synonymous with the term “for example.”

### BRIEF DESCRIPTION OF THE DRAWINGS

This application file contains at least one drawing executed in color. Copies of this patent application publication with color drawings will be provided by the Office upon request and payment of the necessary fee.

FIG. 1 shows summary of specific aims. APC, antigen-presenting cell; MEW, major histocompatibility complex; TCR, T cell receptor.

FIGS. 2A and 2B show intact development of (FIG. 2A) thymocytes and (FIG. 2B) T cells in Cd4CreLrrc8a^{f/f} (cKO) mice. DN, CD4⁻CD8″ double negative; DP, CD4⁺CD8⁺ double positive; SP, CD4⁺ or CD8⁺ single positive. Naïve, CD44^{lo}CD62L^{hi}; CM, CD44^{hi}CD62L^{hi} central memory; Ef/EM, CD44^{hi}CD62L^{lo} effector/memory.

FIG. 3A-3C show impaired 2° activation in cKO-OTII CD4⁺T cells. A. Methods of primary (1°) and secondary (2°) activation. FIGS. 3B and 3C. WT-OTII vs cKO-OTII CD4⁺ T cell proliferation (FIG. 3B) or IFN-γ secretion (FIG. 3C) after primary or secondary activation. N=6 mice/genotype. **p<0.01, ***p<0.001, 1-way ANOVA, Holm-Šídák post-hoc test.

FIG. 4A-4C show LRRC8A regulates gene expression downstream of the TCR. Antigen-primed WT-OTII vs cKO-OTII CD4⁺ T cells were re-stimulated with IFN-γ-treated fibroblasts as in FIG. 3A. FIG. 4A. Pathways of downregulated genes in cKO-OTII CD4⁺ T cells. FIG. 4B, 4C. Expression of genes downstream of the TCR (FIG. 4B), CD28, and ICOS (FIG. 4C). n=7 mice per genotype.

FIG. 5A-5D show conditional deletion of LRRC8A impairs granzyme B expression and cytotoxicity in CD8⁺ T cells. FIG. 5A. Methods. FIG. 5B. WT-OTI vs cKO-OTI CD8⁺ T cell proliferation after 1° activation. FIG. 5C. Intracellular IFN-γ and granzyme B after 1° activation for 1 or 3 days, respectively. ***p<0.001 by 1-way ANOVA with Holm-Šídák post-hoc test. FIG. 5D. Cytotoxicity of Ag-primed WT-OTI or cKO-OTI CD8⁺ T cell against Ova₂₅₇₋₂₆₄-loaded CD4⁺ T cells after 4 hours. **p<0.01 by 2-way ANOVA.

FIGS. 6A and 6B show CD28 compensates for LRRC8A deficiency during 1° activation. FIG. 6A. Proliferation of WT-OTII or cKO-OTII CD4⁺ T cells cultured with Ova₃₂₃₋₃₂₉, WT B cells±CTLA4-Ig or isotype for 3 days. FIG. 6B. Proliferation of WT-OTII or cKO-OTII CD4⁺ T cells to crosslinked α-CD3±α-CD28. % divided, >2 divisions by dye dilution. N=2 mice per genotype. **p<0.01 by 1-way ANOVA with Holm-Šídák post-hoc test.

FIG. 7A-7C show that on T cells, LRRC8A has an extracellular C-terminus and colocalizes with CD3ε. FIGS. 7A and 7B. L1 binding LRRC8A is detected in intact and permeabilized HEK293T cells and fibroblasts, but only in permeabilized T cells. FIG. 7C. PLA of LRRC8A and CD3ε in WT, but not cKO, resting T cells. Quantification of 100 cells per genotype in 2 experiments, ***p<0.001 by Student's t test.

FIG. 8A-8D show LRRC8A is necessary for immunity to T-dependent antigens. WT and cKO mice were immunized (imm) with TNP-KLH on days 0 and 14. Draining lymph nodes harvested on specified days. FIG. 8A. B220⁺GL7⁺Fas⁺ GC B cells on day 7. FIG. 8B. α-KLH IgG on day 21. FIG. 8C. T cell proliferation to KLH on day 21. FIG. 8D. CD4⁺CXCR5⁺PD1⁺ T_{FH} cells on day 7. FIG. 8C. n=7 mice/genotype; *p<0.05 by t test; ***p<0.001 by 1-way ANOVA with Holm-Šídák post-hoc test.

FIG. 9A-9C show LRRC8A is required for mitochondrial function in antigen-primed CD8⁺ and CD4⁺ T cells. FIG. 9A. In vitro differentiation of CD8⁺CD44⁺ T cells was comparable from WT-OTI and cKO-OTI CD8⁺ T cells. FIG. 9B. Extracellular flux analysis of purified WT-OTI vs cKO-OTI CD8⁺CD44⁺ T cells. FIG. 9C. Extracellular flux analysis of WT-OTII vs cKO-OTII CD4⁺ T cells after 2° activation for 3 days. *p<0.05, **p<0.01, ***p<0.001 by 1-way ANOVA with Holm-Šídák post-hoc test.

FIG. 10 shows immunoblot of LRRC8A in lysates from T cells (total) and purified mitochondria (Mito). Mitochondrial purity was confirmed by the presence of the mitochondrial protein COX IV and the absence of proteins in other subcellular fractions.

FIG. 11 show conditional LRRC8A deficiency impairs glycolysis in antigen-experienced CD4⁺ T cells during 2° activation. 2° activation of WT-OTII vs cKO-OTII CD4⁺ T cells was performed as in FIG. 3A. The conversion of glucose to lactate releases protons from cells, indicated by the extracellular acidification rate (ECAR). The addition of 10 mM glucose (Glc) reveals the capacity for catabolizing saturating (10 mM) glucose concentrations through glycolysis and aerobic respiration (1). The mitochondrial inhibitor oligomycin causes a compensatory increase in glycolysis, revealing the maximum glycolytic capacity (2). 2-deoxy-glucose (2-DG) inhibits glycolysis, indicating the rate of non-glycolytic acidification (3). N=2 mice per genotype, *p<0.05, **p<0.01 by 1-way ANOVA with Holm-Šídák post-hoc test.

FIG. 12 shows glucose restriction conditions for testing metabolic flexibility.

FIG. 13A-13C show conditional deletion of LRRC8A in donor CD4⁺ T cells reduces acute GVHD. FIG. 13A. Transplantation methods. FIG. 13B. Kaplan Meier survival curve. ***p<0.001 by 2-way ANOVA. FIG. 13C. Serum IFN-γ 7 days and weight loss 14 days after transplantation. ud, undetectable. N=10 mice per genotype. **p<0.01, ***p<0.001 by 1-way ANOVA.

FIG. 14A show histologic scores and representative sections of recipient colons 14 days post-transplantation. Scale, 100 px. FIG. 14B show numbers of total CD4⁺ and CD4⁺CD62¹⁰CD44^{hi} effector/effector memory T cells in the lamina propria. Numbers of lamina propria CD4⁺ T cells positive for the indicated intracellular cytokines. *p<0.05, **p<0.01,***p<0.001 by 1-way ANOVA with the Holm-Šídák post-hoc test. N=10 mice per genotype.

FIGS. 15A and 15B show cKO mice have intact regulatory T cell (Treg) percentages and function. FIG. 15A. Percentages of CD4⁺FOXP3⁺ Treg cells in WT vs cKO spleens. FIG. 15B. In vitro suppression assay of anti-CD3-stimulated WT T effector cells by WT vs cKO CD4⁺CD25⁺CD39⁺ Treg cells.

FIG. 16 show proposed approach for investigating how LRRC8A modulates diabetes induced by self-reactive CD4⁺ and CD8⁺ T cells.

FIGS. 17A and 17 B show cKO T cells have intact mitochondrial function before and after 1° activation. Extracellular flux analysis of oxygen consumption rate (OCR). Oligomycin (Olig) inhibits the mitochondrial ATP synthase, revealing decreased OCR equivalent to basal cellular respiration (1). Carbonyl cyanide-4(trifluoromethoxy) phenylhydrazone (FCCP) ablates the inner mitochondrial membrane potential, maximizing electron flow through the electron transport chain and OCR (2). The difference between the maximal and basal OCR (3) corresponds to the spare respiratory capacity (SRC) for generating ATP during increased energy demands. Rotenone and actinomycin (R+A) abrogate mitochondrial respiration, revealing the rate of non-mitochondrial respiration (4). FIG. 17A. OCR of purified WT-OTI vs cKO-OTI CD8⁺ T cells before and after 1 hour of 1° stimulation with OVA₂₅₇₋₂₆₄-loaded WT B cells. FIG. 17B. OCR of purified WT-OTII vs cKO-OTII CD4⁺ T cells at rest and after 1 day of stimulation with OVA₃₂₃₋₃₃₉-loaded WT B cells. Time points shown are maximal OCR during 1° activation.

FIG. 18 shows surface staining on non-permeabilized cells using an anti-LRRC8A antibody directed against the loop between the first and second transmembrane regions (L1). Wild-type OTII T cell proliferation in response to Ova peptide presented by IFN-g treated fibroblasts (lower left) vs T cell-depleted splenocytes (lower right), in the presence or absence of isotype control, anti-LRRC8A L1 pAb, or anti-LRRC8A C14 pAb.

FIG. 19A-FIG. 19J show that LRRC8A promotes the optimal primary activation of CD4⁺ T cells. FIG. 19A. Immunoblot of LRRC8A in WT and Cd4-Cre Lrrc8a^{fl/fl} T and B cells, representative of 8 experiments. FIG. 19B. Numbers of thymocytes and splenic T cells from WT and Cd4-Cre Lrrc8a^{fl/fl} mice. n=4/genotype, representative of 4 experiments. FIG. 19C. Quantification of proximity ligation assay signal between LRRC8A and CD3ε on Cd4-Cre and Cd4-Cre Lrrc8a^{fl/fl} CD4⁺ T cells. Each graphed point corresponds to the average number of PLA spots per nucleus in a field of 100 cells. Data representative of four experiments. FIG. 19D-FIG. 19F. Mean fluorescence intensity (MFI) of FIG. 19D CD25, (FIG. 19E) CD44, (FIG. 19F) granzyme B (GzmB) of WT-OTII or Cd4-Cre Lrrc8a^{fl/fl}-OTII CD4⁺ T cells after activation with OVA₃₂₃₋₃₂₉ peptide (OVAp) on B cells. (FIG. 19H-FIG. 19I) Mixed lymphocyte reaction of C57BL/6J WT or Cd4-Cre Lrrc8a^{fl/fl} CD4⁺ T cells, stained with CellTrace™ Violet (CTV), and mitomycin c-treated FVB splenocytes. Alloreactive (Allo+) cells proliferate and become CTV^{low}, while non-responding cells (Allo−) remain CTV^{hi}. Cells were assessed for (FIG. 19H) CD44 and (FIG. 19I) GzmB expression. (FIG. 19I-FIG. 19J) Mixed lymphocyte reaction of CTV-loaded WT or ebo CD4⁺ T cells and mitomycin c-treated C57BL/6J splenocytes, followed by staining for (FIG. 19J) CD44 and GzmB. For (FIG. 19G-FIG. 19J), n=3-4/genotype pooled from 2 experiments *p<0.05, **p<0.01, ***p<0.001 by the Holm-Šídák test.

FIG. 20-20E shows that LRRC8A is essential for the secondary CD4⁺ T cell activation. (FIG. 20A) Schematic of WT or Cd4-Cre Lrrc8a^{fl/fl} OTII CD4⁺ T cells undergoing primary activation, followed by secondary activation by hematopoietic APCs (B cells) or non-hematopoietic APCs (IFN-γ-treated fibroblasts). (FIG. 20B-FIG. 20D) (FIG. 20B) Proliferation, (FIG. 20C) survival, and (FIG. 20D) IFN-γ secretion from WT-OTII or Cd4-Cre Lrrc8a^{fl/fl}-OTII CD4⁺ T cells after secondary activation by splenic B cells. n=3/genotype, representative of four independent experiments. (FIG. 20E-FIG. 20E) (FIG. 20E) Proliferation survival, and IFN-γ secretion of CD4⁺ T cells after secondary activation by IFN-γ-treated fibroblasts. n=4/genotype, representative of six experiments. **p<0.01, ***p<0.001 by the Holm-Šídák test.

FIG. 21A-21H shows the selective deletion of LRRC8A in T cells impairs the TCR-driven transcriptional signature. WT-OTII or Cd4-Cre Lrrc8a^{fl/fl}-OTII CD4⁺ T cells underwent secondary activation by OVAp-presenting fibroblasts as in FIG. 2A. (FIG. 21A) Volcano plot with significantly upregulated (red) and downregulated (blue) genes in re-activated Cd4-Cre Lrrc8a^{fl/fl}-OTII CD4⁺ T cells, compared to controls. A false discovery rate (FDR)-adjusted p<0.05 and log₂ fold change ≥2 were considered significant. n=5/genotype from 2 experiments. (FIG. 21B) Pathways enriched for differentially expressed genes in re-activated WT-OTII and Cd4-Cre Lrrc8a^{fl/fl}-OTII CD4⁺ T cells. (FIG. 21C) Heatmap of genes downstream of the T cell receptor, determined by Ingenuity Pathway Analysis, in reactivated WT-OTII or Cd4-Cre Lrrc8a^{fl/fl}-OTII CD4⁺ T cells. (FIG. 21D) Protein expression of GzmB, CD25, and CCR4 and qPCR of Nur77, normalized to Hprt, in re-activated WT-OTII or Cd4-Cre Lrrc8a^{fl/fl}-OTII CD4⁺ T cells. n=4/genotype from 2 experiments (FIG. 21E, FIG. 21F) (FIG. 21E) Mitochondrial mass (Mitotracker Green, MTG), mitochondrial potential (CMXRos), and (FIG. 21F) mitochondrial respiration in re-activated WT-OTII or Cd4-Cre Lrrc8a^{fl/fl}-OTII CD4⁺ T cells. OCR, oxygen consumption rate. (FIG. 21G, FIG. 21H) (FIG. 21G) Protein expression of Glut1 and Glut3 and (FIG. 21H) glycolytic function in re-activated WT-OTII or Cd4-Cre Lrrc8a^{fl/fl}-OTII CD4⁺ T cells. ECAR, extracellular acidification rate. Data in (FIG. 21E-FIG. 21H) are representative of 4 experiments, each with 4 mice/genotype. *p<0.05, **p<0.01, ***p<0.001 by the Holm-Šídák test.

FIG. 22A-22F shows the selective deletion of LRRC8A in donor CD4⁺ T cells attenuates acute GVHD from mismatched minor histocompatibility antigens. (FIG. 22A) Schematic of transplantation model. (FIG. 22B) Combined survival from three experiments; n=10/genotype. ***p<0.001 by two-way ANOVA. (FIG. 22C) Serum IFN-γ seven days after transplantation. n=6/genotype pooled from 2 experiments. (FIG. 22D) Histologic scoring and H&E staining of recipient colons 14 days after transplantation. Scale, 26 mm. Images are representative of 10 mice per genotype. (FIG. 22E-FIG. 22F) Flow cytometric analysis of CD4⁺ T cells isolated from the lamina propria of recipients 14 days after transplantation. CD4⁺ effector/memory T cells were identified as CD4⁺CD62L^{lo}CD44⁺ T cells. For (FIG. 22B, FIG. 22E-FIG. 22F), n=6/genotype pooled from two experiments and representative of four experiments. *p<0.05, **p<0.01, ***p<0.001 by the Holm-Šídák test.

FIG. 23A-FIG. 23H depict the characterization of T cell development and function in mice with conditional deletion of LRRC8A in T cells (FIG. 23A) Schematic showing the generation of Cd4-Cre Lrrc8a^{fl/fl} mice. In the targeting Lrrc8a vector (Lac8a^{tm2a(EUCOMM)Hmgu}), FRT sites flank the bacterial lacZ gene and neomycin resistance gene (Neo) under the control of the human beta-actin promoter; loxP sites flank exon 3 of Lrrc8a, which encodes 719 of the protein's 811 amino acids. Cd4-Cre Lrrc8a^{fl/fl} mice were generated by breeding mice with Lrrc8a^{tm2a(EUCOMM)Hmgu} allele FLP1 recombinase with transgenic mice, followed by breeding with Cd4-Cre recombinase transgenic mice. (FIG. 23B, FIG. 23C) Flow cytometry plots of (FIG. 23A) thymocytes and (FIG. 23B) splenic T cells from Cd4-Cre and Cd4-Cre Lrrc8a^{fl/fl} mice, representative of eight experiments. (FIG. 23C) Numbers of double negative (DN)1-DN4 thymocytes and naïve, central memory (CM), and effector memory (EM) CD8⁺ splenic T cell subpopulations from Cd4-Cre and Cd4-Cre Lrrc8a^{fl/fl} mice. n=4/genotype, pooled from two experiments. (FIG. 23D) Representative image of proximal ligation assay using Cd4-Cre and Cd4-Cre Lrrc8a^{fl/fl} CD4⁺ T cells. TRITC, fluorescent DNA templates formed between proteins <30-40 nm apart; DAPI nuclear stain shown in blue. (FIG. 23E-FIG. 23H) CD4⁺ T cells from WT-OTII and Cd4-Cre Lrrc8a^{fl/fl}-OTII mice underwent primary activation by OVAp-presenting B cells, followed by quantification of (FIG. 23E) CD4⁺C25⁺ T cells, CD4⁺GzmB⁺ T cells, (FIG. 23F) CD4⁺C44⁺ T cells, (FIG. 23G) proliferation, (FIG. 23H) IFN-γ secretion. Percentages shown are gated on the CD4⁺ T cell population. n=4/genotype, pooled from two experiments. ***p<0.001 by the Holm-Šídák test.

FIG. 24A-FIG. 24D show that LRRC8A preserves optimal CD25 activation independently of its channel activity. (FIG. 24A, FIG. 24B) Mixed lymphocyte reaction of Cd4-Cre (WT) and Cd4-Cre Lrrc8a^{fl/fl} T cells, loaded with CellTrace™ Violet (CTV), and MHC-mismatched, mitomycin c-treated FVB/NJ splenocytes, followed by assessment of (FIG. 24A) CD25⁺ expression in alloreactive (Allo+) cells and non-responding cells (Allo−) and (FIG. 24B) proliferation. (FIG. 24C, FIG. 24D) Mixed lymphocyte reaction of CTV-loaded WT or ebo CD4⁺ T cells and MHC-mismatched, mitomycin c-treated C57BL/6J splenocytes, followed by assessment of (FIG. 24C) CD25 expression and (FIG. 24D) proliferation, n=3-4/genotype pooled from two experiments. The ebo mice express a truncated form of LRRC8A that lacks channel activity (17). ***p<0.001 by the Holm-Šídák test.

FIG. 25A-FIG. 25C show that the conditional deletion of Lrrc8a in T cells impairs mitochondrial biogenesis, potential, and expression of glucose receptors. WT-OTII or Cd4-Cre Lrrc8a^{fl/fl} OTII CD4⁺ T cells underwent secondary activation by OVAp-presenting fibroblasts as in FIG. 2A, followed by assessment of (FIG. 25A) mitochondrial mass (Mitotracker Green, MTG), (FIG. 25B) mitochondrial membrane potential (chloromethyl-X-rosamine, CMXRos), and (FIG. 25C) Glut1 and Glut3 expression. Histograms are representative of four independent experiments.

FIG. 26A and FIG. 26B show that Cd4-Cre Lrrc8a^{fl/fl} mice have intact percentages and suppressor function of regulatory T cells. (FIG. 26A) CD4⁺FOXP3⁺ regulatory T cell (Treg) percentages in the bone marrow from Cd4-Cre or Cd4-Cre Lrrc8a^{fl/fl} mice. (FIG. 26B) Suppression assay using Tregs from Cd4-Cre or Cd4-Cre Lrrc8a^{fl/fl} mice.

FIG. 27A-FIG. 27C show that the conditional deletion of LRRC8A in donor CD4+ T cells permits survival in a mouse model of lethal acute GVHD. (FIG. 27A) In this model, either wild-type (WT) or LRRC8A-deficient (cKO) CD4+ donor T cells (C57BL/6 background, 3×10⁶ cells of each genotype) are infused with T cell-depleted bone marrow into an irradiated Balb/c recipient. This is a model of acute GVHD arising from mismatched MHC I and MHC II antigens, resulting in 100% mortality by Day 7 when WT donor T cells are infused. In contrast, 100% of recipients receiving LRRC8A-deficient donor T cells survived. (FIG. 27B) Recipients of either WT or cKO donor CD4+ T cells had equivalent numbers of donor CD4+ T cells in the spleen. Thus, the survival of recipients of cKO donor CD4+ T cells was not due to premature death of donor T cells. (FIG. 27C) Recipients of cKO donor CD4+ T cells had reduced expression of effector proteins and cytokines important for the pathogenesis of acute GVHD: Granzyme B (GzmB), interferon gamma (IFN-γ), and interleukin 6 (IL-6). This demonstrates that the deletion of LRRC8A from CD4+ donor T cells impairs protein expression known to be downstream of T cell activation.

FIG. 28A and FIG. 28B show that the addition of the anti-LRRC8A antibody (C14) impairs the mixed lymphocyte reaction, and that an in vitro assay correlates with the severity of acute GVHD. In this model, irradiated Balb/c splenocytes are cultured for seven days with donor CD4+ T cells from WT mice and either an isotype IgG (Iso) as a negative control or the anti-LRRC8A antibody C14. (FIG. 28A) In this well-established in vitro assay, the degree of donor CD4+ T cell activation, demonstrated by the expression of effector proteins such as granzyme B (GzmB), is known to correlate with the severity of acute GVHD. Viability of cultures with the isotype control and C14 are comparable, indicating that the C14 antibody has no detrimental effect on the survival of donor CD4+ T cells. (FIG. 28B) The addition of C14 significantly reduces the expression of GzmB, a protein indicative of sustained CD4+ T cell activation. These result complement FIG. 18 in the provisional patent by using an in vitro model that is well known to correlate with acute GVHD in vivo.

FIG. 29A and FIG. 29B show that the addition of the anti-LRRC8A antibody (C14) impairs the mixed lymphocyte reaction using human cells. A mixed lymphocyte reaction was set up between MHC Class I and Class II human donors and recipients (3 different individuals), with either isotype control or the C14 antibody. (FIG. 29A) As seen with mouse cells, the C14 antibody has no detrimental effect on the survival of human donor CD4+ T cells. (FIG. 29B) The addition of C14 significantly reduces the expression of CD25, a marker used in clinical medicine to measure the level of T cell activation, which correlates with acute GVHD and the inflammatory response.

### DETAILED DESCRIPTION

### Treating or Preventing GVHD

Demonstrated herein is a requirement for the transmembrane protein LRRC8A in thymocyte development.³ Using a conditional knockout model that bypasses the requirement for LRRC8A in thymocyte development, it was shown that LRRC8A is essential for the activation and metabolism of antigen-experienced T cells. LRRC8A also functions as the pore-forming subunit of the volume-regulated anion channel in the plasma membrane, which gates anion and osmolyte efflux in response to hypo-osmotic stress.^{4,5}

It is further shown that LRRC8A contributes to the primary activation of CD4+ T cells and is essential for the secondary response of antigen-experienced CD4+ effector T cells. Conditional deletion of LRRC8A in T cells impaired the generation of germinal center B cells and antibody responses to T-dependent antigens. Additionally, LRRC8A-deficient CD8+ T cells had impaired expression of granzyme B after primary activation and defective cytolytic activity during secondary stimulation with antigen-loaded targets. LRRC8A activates pathways downstream of the TCR, CD28, and ICOS and promotes the expression of genes important for glycolysis. Notably, it was found that LRRC8A localizes to the mitochondria in addition to the plasma membrane. A C-terminal fragment of LRRC8A binds to electron transfer flavoprotein and ATP synthase, two components of the mitochondrial electron transport chain. Conditional deletion of LRRC8A in T cells impaired the mitochondrial oxidative function of antigen-primed CD4+ and CD8+ T cells. Identified herein is a role for LRRC8A in CD4+ T cell-driven, acute GVHD arising from mismatched minor histocompatibility antigens. Donor CD4+ T cells lacking LRRC8A had reduced expression of inflammatory cytokines and induced minimal gastrointestinal pathology, thus demonstrating the biologic relevance of LRRC8A-driven CD4+ T cell activation. Data presented herein show blocking LRRC8A using an antibody specific for the C-terminus of LRRC8A inhibits activation of mouse or human T cells stimulated by the mixed lymphocyte reaction, an in vitro assay that predicts that severity of acute GVHD.

One aspect of the invention is for treating or preventing graft versus host disease, the method comprising administering to a subject having, or at risk of developing, graft versus host disease (GVHD) an agent that inhibits LRRC8A. GVHD can be acute or chronic.

Another aspect of the invention is a method for treating or preventing graft versus host disease comprising administering to a subject in need thereof an agent that inhibits LRRC8A, wherein the agent is an antibody binds an antigen having a sequence selected from the group consisting of: SEQ ID NO: 3 and SEQ ID NO: 4.

As used herein, an “GVHD” refers to a disease characterized by the active process of donor cells attacking the recipient's own cells. GVHD can develop soon after a transplant, e.g., within weeks or months (acute GVHD), or can occur much later after the transplant, e.g., at least 3-6 months later (chronic GVHD). Symptoms of acute GVHD include, but are not limited to, skin rash or blisters, abdominal pain or discomfort, diarrhea, jaundice, and edema. Symptoms of chronic GVHD include, but are not limited to, changes to skin or nail texture, hair loss or thinning, muscle pain or weakness, blurred vision, mouth sores, shortness of breath, persistent cough, abdominal pain or discomfort, and diarrhea.

In one embodiment, an agent that inhibits LRRC8A is administered as a prophylactic treatment to prevent GVHD in a subject at risk of developing GVHD, for example, following an organ or tissue transplant. Risk factors for developing GVHD, include but are not limited to a subject or donor of advanced age (for example, an age greater that 60), female subject who has had a previous pregnancy, donor-recipient disparity in human leukocyte antigen (HLA) haplotypes or gender, source of transplant material, dosage and choice of conditioning, chemotherapeutic, immunosuppressive, and antimicrobial agents in the peri-transplantation period, pre-existing immune dysregulation, splenectomy, prior history of infections, and history of pre- and/or post-transplant blood transfusions.

In one embodiment, the subject has received an organ transplant or a hematopoietic stem cell transplant. In one embodiment, the subject has received an organ transplant or a hematopoietic stem cell transplant at least 1, 2, 3, 4, 5, 6 days, or 1, 2, 3 weeks, or 1, 2, 3, 4, 5, 6, 7, 8, 9, 10, 11 months, or 1 year, or more prior to administration. In another embodiment, the agent is administered prior to, during, or immediately following (e.g., during the initial recovery of) the organ transplant or a hematopoietic stem cell transplant.

In one embodiment, the method further comprises, prior to administering, the step of diagnosing a subject as having, or at risk of developing, graft versus host disease. In another embodiment, the method further comprises, prior to administering, the step of receiving the results from an assay that identifies a subject as having graft versus host disease. A subject can be identified as having or be at risk of having GVHD by a skilled clinician. Diagnostic tests useful in identifying a subject having GVHD are known in the art and will vary based on the type of transplant the subject has received. The diagnosis of GVHD is made by, for example, physical examination for the signs and symptoms for GVHD known in the art, serologic testing for dysfunction of the liver, gall bladder, kidney, and hematopoietic cells, histologic analysis of biopsies obtained from affected organs, and radiologic imaging of affected organs. In one embodiment, the method further comprises administering at least a second therapeutic. In one embodiment, the agent described herein that inhibits LRRC8A is administered in combination with Abatacept (Orencia®) or Belatacept (Nulojix®). Abatacept and Belatacept, developed by Bristol-Meyers Squibb, are fusion proteins composed of the Fc region of the immunoglobulin IgG1 fused to the extracellular domain of CTLA-4. Abatacept is currently approved by the FDA for treatment of rheumatoid arthritis. Belatacept, which only differs from Abatacept by two amino acids, is an immunosuppressant intended to prevent rejection following a kidney transplant.

### Treating or Preventing Diabetes

Demonstrated herein is a requirement for the transmembrane protein LRRC8A in CD8+ T cell function. CD4+ and CD8+ T cells are known to be important contributors to the development of Type 1 and Type 2 diabetes. In particular, the oxidative metabolism of T cells, controlled by the mitochondria, is a driver of T cell-driven pancreatic destruction. LRRC8A-deficient CD8+ T cells had impaired expression of granzyme B after primary activation and defective cytolytic activity during secondary stimulation with antigen-loaded targets. Using a conditional knockout model that bypasses the requirement for LRRC8A in thymocyte development, it was shown that LRRC8A is essential for the metabolism of antigen-experienced T cells. Notably, it was found that LRRC8A localizes to the mitochondria in addition to the plasma membrane. A C-terminal fragment of LRRC8A binds to electron transfer flavoprotein and ATP synthase, two components of the mitochondrial electron transport chain. Conditional deletion of LRRC8A in T cells impaired the mitochondrial oxidative function of antigen-primed CD4+ and CD8+ T cells. Additionally, LRRC8A-deficient CD4+ T cells have impaired expression of genes important for glycolysis and demonstrate reduced glycolytic function. Identified herein is the requirement for LRRC8A in the activation of CD4+ T cells by antigen-presenting cells. LRRC8A-deficient CD4+ T cells manifested reduced proliferation and secretion of the inflammatory cytokine interferon-gamma in response to antigen presented by fibroblasts.

One aspect of the invention is for treating diabetes, the method comprising administering to a subject in need thereof an agent that inhibits LRRC8A. In one embodiment, diabetes is type I diabetes. In another embodiment, diabetes is type 2 diabetes.

Another aspect of the invention is a method for treating diabetes comprising administering to a subject in need thereof an agent that inhibits LRRC8A, wherein the agent is an antibody binds an antigen selected from the group consisting of: SEQ ID NO: 3 and SEQ ID NO: 4.

As used herein, “type I diabetes (T1D)” refers to a disease characterized the inability for a pancreatic cell to produce insulin. T1D is caused by an autoimmune reaction (the body attacks itself by mistake) that destroys the cells in the pancreas that make insulin, called beta cells. This process can go on for months or years before any symptoms appear. Some subjects have certain genes that make them more likely to develop T1D, though many won't go on to have TID even if they have the genes. Being exposed to a trigger in the environment, such as a virus, is also thought to play a part in developing T1D. Diet and lifestyle habits do not cause the onset of T1D. Symptoms of chronic T1D include, but are not limited to, excessive thirst, fatigue, persistent hunger, persistent sweating, nausea and vomiting, excessive urination, blurred vision, rapid heart rate, headaches, restlessness, and unexplained weight loss. The requirement for CD8+ T cell activation in the pathogenesis of Type I diabetes is known in the art. The cytotoxicity of CD8+ T cells infiltrating the pancreas lead to organ destruction and the development of diabetes.

As used herein, “type 2 diabetes (T2D) refers to a disease characterized by high blood sugar, insulin resistance, and lack of insulin relative to a healthy individual (e.g., and individual not having T2D). T2D is due to insufficient insulin production from beta cells in the setting of insulin resistance. Insulin resistance is a pathological condition in which cells fail to respond normally to insulin. Common symptoms of T2D include, but are not limited to, frequent urination, increased thirst, increased hunger, unexplained weight loss, blurred vision, itchiness, peripheral neuropathy, recurrent vaginal infections in females, and fatigue. Subjects at risk of having or developing T2D include those subjects who are overweight or obese (e.g., a body-mass index greater than 25), increased stress levels, poor diet (e.g., increased sugar consumption), lack of exercise, of advanced age, female, have T1D, or have or have had gestational diabetes (diabetes during, and caused by, a pregnancy). CD4+ T cell activation contributes to the pathogenesis of T2D. The contribution of T cell metabolism in the pathogenesis of T2D is known in the art, as demonstrated by the extensive use of metformin, an inhibitor of oxidative phosphorylation and mitochondrial function, in the treatment of patients with T2D.

In one embodiment, an agent that inhibits LRRC8A is administered as a prophalytic treatment to prevent diabetes in a subject at risk of developing diabetes, for example. Risk factors for developing diabetes, include but are not limited family history of diabetes, race and ethnicity (Caucasians are more susceptible that other ethnicities), geography, and exposure to certain viral infections.

In one embodiment, the method further comprises, prior to administering, the step of diagnosing a subject as having diabetes. In another embodiment, the method further comprises, prior to administering, the step of receiving the results from an assay that identifies a subject as having diabetes. A subject can be identified as having or at risk of developing diabetes by a skilled clinician. Diagnostic tests useful in identifying a subject having diabetes are known in the art.

In one embodiment, the method further comprises administering at least a second therapeutic. In one embodiment, the agent described herein that inhibits LRRC8A is administered in combination with insulin, Abatacept (Orencia®) or Belatacept (Nulojix®). Insulin replacement therapy is the most common treatment for managing T1D and T2D.

### Agents

In one aspect, an agent that inhibits LRRC8A is administered to a subject having, or at risk of developing GVHD or diabetes (e.g., T1D or T2D). In one embodiment, the agent that inhibits LRRC8A is a small molecule, an antibody or antibody fragment, a peptide, an antisense oligonucleotide, a genome editing system, or an RNAi.

In one embodiment, the agent described herein can inhibit the level or activity of LRRC8A. An agent is considered effective for inhibiting LRRC8A if, for example, upon administration, it inhibits the presence, amount, activity and/or level of LRRC8A in the cell. In one embodiment, the agent inhibits LRRC8A on a T cell or antigen presenting cell. In one embodiment, inhibits LRRC8A on at least 5%, 10%, 15%, 20%, 25%, 30%, 35%, 40%, 45%, 50%, 55%, 60%, 65%, 70%, 75%, 80%, 85%, 90% 95%, 99%, or more of T cells or antigen presenting cell in the subject. A skilled practitioner will be able to determine if LRRC8A is inhibited on a T cell or antigen presenting cell, for example, by sorting T cells or antigen presenting cell via flow cytometry and assessing the level of LRRC8A protein or mRNA via western blotting or PCR-based assays, respectively.

An agent can inhibit e.g., the transcription, or the translation of LRRC8A in the cell. An agent can inhibit the activity (e.g., promoting the function of volume-regulated anion channel) or alter the activity (e.g., such that the activity no longer occurs, or occurs at a reduced rate) of LRRC8A in the cell (e.g., LRRC8A's expression).

In one embodiment, an agent that inhibits LRRC8A inhibits the expression level or activity of LRRC8A. To determine if an agent is effective at inhibiting LRRC8A levels, mRNA and protein levels of a given target (e.g., LRRC8A) can be assessed using RT-PCR and western-blotting, respectively. Biological assays that detect the activity LRRC8A, for example promoting the function of volume-regulated anion channel, are known in the art can be used to assess if a decrease in LRRC8A activity has occurred following administration of the agent.

In one embodiment, an agent that inhibits the level and/or activity (e.g., promoting the function of volume-regulated anion channel) of LRRC8A by at least 10%, by at least 20%, by at least 30%, by at least 40%, by at least 50%, by at least 60%, by at least 70%, by at least 80%, by at least 90%, by at least 100% or more as compared to an appropriate control. As used herein, an “appropriate control” refers to the level and/or activity of LRRC8A prior to administration of the agent, or the level and/or activity of LRRC8A in a population of cells that was not in contact with the agent.

The agent may function directly in the form in which it is administered. Alternatively, the agent can be modified or utilized intracellularly to produce something which inhibits LRRC8A, such as introduction of a nucleic acid sequence into the cell and its transcription resulting in the production of the nucleic acid and/or protein inhibitor of LRRC8A. In some embodiments, the agent is any chemical, entity or moiety, including without limitation synthetic and naturally-occurring non-proteinaceous entities. In certain embodiments the agent is a small molecule having a chemical moiety. For example, chemical moieties included unsubstituted or substituted alkyl, aromatic, or heterocyclyl moieties including macrolides, leptomycins and related natural products or analogues thereof. Agents can be known to have a desired activity and/or property, or can be identified from a library of diverse compounds.

In various embodiments, the agent is a small molecule that inhibits LRRC8A. Methods for screening small molecules are known in the art and can be used to identify a small molecule that is efficient at, for example, inhibiting the function of volume-regulated anion channel

In various embodiments, the agent that inhibits LRRC8A is an antibody or antigen-binding fragment thereof, or an antibody reagent that is specific for LRRC8A. As used herein, the term “antibody reagent” refers to a polypeptide that includes at least one immunoglobulin variable domain or immunoglobulin variable domain sequence and which specifically binds a given antigen. An antibody reagent can comprise an antibody or a polypeptide comprising an antigen-binding domain of an antibody. In some embodiments of any of the aspects, an antibody reagent can comprise a monoclonal antibody or a polypeptide comprising an antigen-binding domain of a monoclonal antibody. For example, an antibody can include a heavy (H) chain variable region (abbreviated herein as VH), and a light (L) chain variable region (abbreviated herein as VL). In another example, an antibody includes two heavy (H) chain variable regions and two light (L) chain variable regions. The term “antibody reagent” encompasses antigen-binding fragments of antibodies (e.g., single chain antibodies, Fab and sFab fragments, F(ab′)2, Fd fragments, Fv fragments, scFv, CDRs, and domain antibody (dAb) fragments (see, e.g. de Wildt et al., Eur J. Immunol. 1996; 26(3):629-39; which is incorporated by reference herein in its entirety)) as well as complete antibodies. An antibody can have the structural features of IgA, IgG, IgE, IgD, or IgM (as well as subtypes and combinations thereof). Antibodies can be from any source, including mouse, rabbit, pig, rat, and primate (human and non-human primate) and primatized antibodies. Antibodies also include midibodies, nanobodies, humanized antibodies, chimeric antibodies, and the like.

In one embodiment, the agent that inhibits LRRC8A is a humanized, monoclonal antibody or antigen-binding fragment thereof, or an antibody reagent. As used herein, “humanized” refers to antibodies from non-human species (e.g., mouse, rat, sheep, etc.) whose protein sequence has been modified such that it increases the similarities to antibody variants produce naturally in humans. In one embodiment, the humanized antibody is a humanized monoclonal antibody. In one embodiment, the humanized antibody is a humanized polyclonal antibody. In one embodiment, the humanized antibody is for therapeutic use. Methods for humanizing a non-human antibody are known in the art.

In one embodiment, the antibody or antibody reagent binds to an amino acid sequence that corresponds to the amino acid sequence encoding LRRC8A (SEQ ID NO: 2).

(SEQ ID NO: 2)
MIPVTELRYFADTQPAYRILKPWWDVFTDYISIVMLMIAVFGGTLQVTQD
KMICLPCKWVTKDSCNDSFRGWAAPGPEPTYPNSTILPTPDTGPTGIKYD
LDRHQYNYVDAVCYENRLHWFAKYFPYLVLLHTLIFLACSNEWFKFPRTS
SKLEHFVSILLKCFDSPWTTRALSETVVEESDPKPAFSKMNGSMDKKSST
VSEDVEATVPMLQRTKSRIEQGIVDRSETGVLDKKEGEQAKALFEKVKKF
RTHVEEGDIVYRLYMRQTIIKVIKFILIICYTVYYVHNIKFDVDCTVDIE
SLTGYRTYRCAHPLATLFKILASFYISLVIFYGLICMYTLWWMLRRSLKK
YSFESIREESSYSDIPDVKNDFAFMLHLIDQYDPLYSKRFAVFLSEVSEN
KLRQLNLNNEWILDKLRQRLTKNAQDKLELHLFMLSGIPDTVFDLVELEV
LKLELIPDVTIPPSIAQLTGLKELWLYHTAAKIEAPALAFLRENLRALHI
KFTDIKEIPLWIYSLKTLEELHLTGNLSAENNRYIVIDGLRELKRLKVLR
LKSNLSKLPQVVTDVGVHLQKLSINNEGTKLIVLNSLKKMANLTELELIR
CDLERIPHSIFSLHNLQEIDLKDNNLKTIEEIISFQHLHRLTCLKLWYNH
IAYIPIQIGNLTNLERLYLNRNKIEKIPTQLFYCRKLRYLDLSHNNLTFL
PADIGLLQNLQNLAITANRIETLPPELFQCRKLRALHLGNNVLQSLPSRV
GELTNLTQIELRGNRLECLPVELGECPLLKRSGLVVEEDLENTLPPEVKE
RLWRADKEQA

In another embodiment, the anti-LRRC8A antibody or antibody reagent binds to an amino acid sequence that comprises the sequence of SEQ ID NO: 2; or binds to an amino acid sequence that comprises a sequence with at least 80%, at least 85%, at least 90%, at least 95%, at least 96%, at least 97%, at least 98%, at least 99% or greater sequence identity to the sequence of SEQ ID NO: 2. In one embodiment, the anti-LRRC8A antibody or antibody reagent binds to an amino acid sequence that comprises the entire sequence of SEQ ID NO: 2. In another embodiment, the antibody or antibody reagent binds to an amino acid sequence that comprises a fragment of the sequence of SEQ ID NO: 2, wherein the fragment is sufficient to bind its target, e.g., LRRC8A, and for example, inhibit the function of volume-regulated anion channel.

In one embodiment, the anti-LRRC8A antibody or antibody reagent is a polyclonal antibody that targets LRRC8A and results in its inhibition. Two anti-LRRC8A polyclonal antibodies were developed. The C14 anti-LRRC8A antibody is directed against amino acid residues of SEQ ID NO: 2 within the protein's C-terminus (peptide sequence: Ac-CEVKERLWRADKEQA-OH; SEQ ID NO: 3) conjugated to KLH-M. The L1 anti-LRRC8A antibody is directed against amino acid residues of SEQ ID NO: 2 in the loop between the first and second transmembrane domains (peptide sequence: LPTPDTGPTGIKYDLDRH; SEQ ID NO: 4) conjugated to KLH-EG. In one embodiment, polyclonal antibody that targets LRRC8A results in blocking an extracellular portion of LRCC8A on a T cell or an antigen presenting cell.

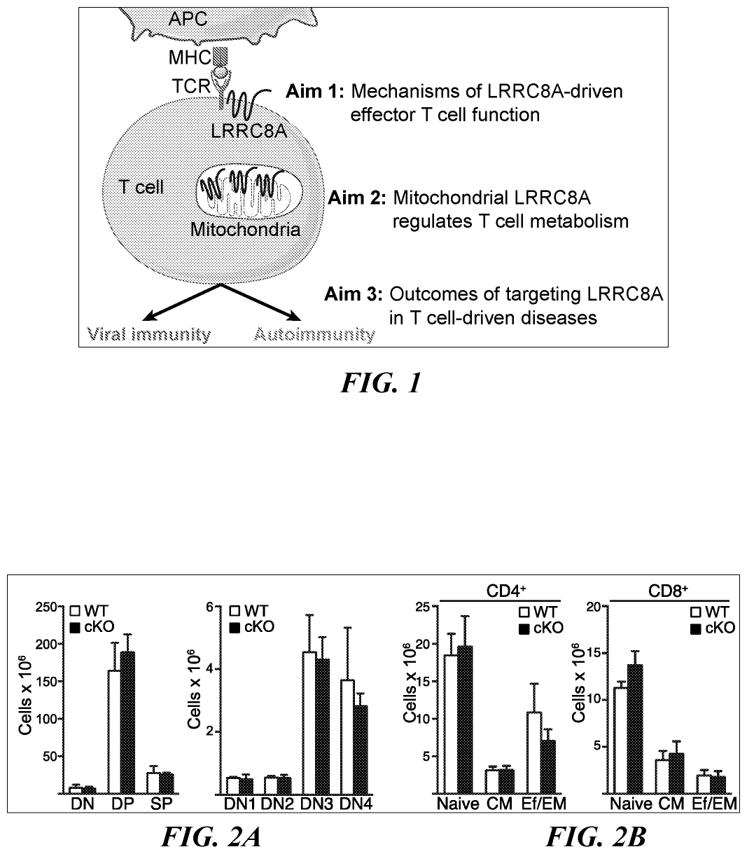

Flow cytometry analysis using L1 to stain multiple cell types showed that the antibody's epitope is extracellular on mouse embryonic fibroblasts, murine keratinocytes, and HEK293T cells, but are intracellular on CD3⁺ T cells and macrophages (FIG. 1 and data not shown). This demonstrates that LRRC8A is a dual topology membrane protein with a cell-specific orientation, indicating that anti-LRRC8A antibodies directed against specific epitopes can differentially target the receptor on hematopoietic vs stromal cells.

The ability of anti-LRRC8A antibodies that target SEQ ID NO: 3 and 4 directed against unique epitopes to block T cell proliferation and activation was tested. T cell proliferation was equally inhibited by the L1 antibody targeting LRRC8A on fibroblasts and the C14 antibody targeting LRRC8A on T cells. The C14 antibody reduced T cell activation, as evidenced by decreased expression of Granzyme B and CD25, but did not inhibit T cell proliferation induced by hematopoietic cells. This has important therapeutic implications because T cell proliferation is critical for host immunity against infectious pathogens. Furthermore, antigen presentation by stromal cells is critical for pathologic T cell activation in acute GVHD. Thus, anti-LRRC8A antibodies can mitigate acute GVHD and still preserve host immunity to infectious organisms. This overcomes a major obstacle associated with many existing immunosuppressive agents, which non-specifically suppress both deleterious and beneficial T cell activation.

Anti-LRRC8A antibodies or antibody reagents are known in the art. For example, such reagents are readily commercially available. In some embodiments of any of the aspects, an antibody or antibody reagent is specific for a target described herein (e.g., that binds specifically to LRRC8A) can be an antibody reagent comprising one or more (e.g., one, two, three, four, five, or six) CDRs of any one of the antibodies recited in Table 7. In some embodiments of any of the aspects, an antibody or antibody reagent specific for a target described herein can be an antibody reagent comprising the six CDRs of any one of the antibodies recited in Table 7. In some embodiments of any of the aspects, an antibody or antibody reagent specific for a target described herein can be an antibody reagent comprising the three heavy chain CDRs of any one of the antibodies recited in Table 7. In some embodiments of any of the aspects, an antibody or antibody reagent specific for a target described can be an antibody reagent comprising the three light chain CDRs of any one of the antibodies recited in Table 7. In some embodiments of any of the aspects, an antibody or antibody reagent specific for a target described herein can be an antibody reagent comprising the VH and/or VL domains of any one of the antibodies recited in Table 7. In some embodiments of any of the aspects, an antibody or antibody reagent specific for a target described herein can be an antibody reagent comprising the VH and VL domains of any one of the antibodies recited in Table 7. In some embodiments of any of the aspects, an antibody or antibody reagent specific for a target described herein can be an antibody reagent recited in Table 7. Such antibody reagents are specifically contemplated for use in the methods and/or compositions described herein.

TABLE 7
Anti-LRRC8A antibodies.

| Antibody Clone | |
|---|---|
| Designation | Source |
| 8H9 | Cat. No. H00056262-M04; Abnova Corporation (Taipei, Taiwan) |
| | Cat. No. DPABH-18123; Creative Diagnostics (Shirley, NY) |
| | (The immunogen used to make this antibody was a peptide corresponding |
| | to residues 2-51 of human LRRC8A (e.g. SEQ ID NO: 1) |
| | Cat. No. DPABH-05870; Creative Diagnostics (Shirley, NY) |
| | (The immunogen used to make this antibody was a peptide corresponding |
| | to residues 648-803 of human LRRC8A (e.g. SEQ ID NO: 1) |
| | Cat. No. LS-C290816-100; LifeSpan BioSciences (Seattle, WA) |
| | (The immunogen used to make this antibody was a peptide corresponding |
| | to residues 650-700 of human LRRC8A (e.g. SEQ ID NO: 1) |
| | Cat. No. orb185053; Biorbyt (Cambridge, United Kingdom) |
| | (The immunogen used to make this antibody was a peptide corresponding |
| | to residues 94-170 of human LRRC8A (e.g. SEQ ID NO: 1) |
| | Cat. No. 70R-6392; Fitzgerald Industries International (Acton, MA) |
| | Cat. No. ab157489; Abcam (Cambridge, United Kingdom) |
| | (The immunogen used to make this antibody was a peptide corresponding |
| | to residues 648-803 of human LRRC8A (e.g. SEQ ID NO: 1) |

In one embodiment, the agent that inhibits LRRC8A is an antisense oligonucleotide. As used herein, an “antisense oligonucleotide” refers to a synthesized nucleic acid sequence that is complementary to a DNA or mRNA sequence, such as that of a microRNA. Antisense oligonucleotides are typically designed to block expression of a DNA or RNA target by binding to the target and halting expression at the level of transcription, translation, or splicing. Antisense oligonucleotides of the present invention are complementary nucleic acid sequences designed to hybridize under cellular conditions to a gene, e.g., LRRC8A. Thus, oligonucleotides are chosen that are sufficiently complementary to the target, i.e., that hybridize sufficiently well and with sufficient specificity in the context of the cellular environment, to give the desired effect. For example, an antisense oligonucleotide that inhibits LRRC8A may comprise at least 5, at least 10, at least 15, at least 20, at least 25, at least 30, or more bases complementary to a portion of the coding sequence of the human LRRC8A gene (e.g., SEQ ID NO: 1).

SEQ ID NO: 1 is a nucleotide sequence that encodes LRRC8A.

(SEQ ID NO: 1)

| | atgattc cggtgacaga |
|---|---|
| 541 | gctccgctac tttgcggaca cgcagccagc ataccggatc |
| | ctgaagccgt ggtgggatgt |
| 601 | gttcacagac tacatctcta tcgtcatgct gatgattgcc |
| | gtcttcgggg ggacgctgca |
| 661 | ggtcacccaa gacaagatga tctgcctgcc ttgtaagtgg |
| | gtcaccaagg actcctgcaa |
| 721 | tgattcgttc cggggctggg cagcccctgg cccggagccc |
| | acctacccca actccaccat |
| 781 | tctgccgacc cctgacacgg gccccacagg catcaagtat |
| | gacctggacc ggcaccagta |
| 841 | caactacgtg gacgctgtgt gctatgagaa ccgactgcac |
| | tggtttgcca agtacttccc |
| 901 | ctacctggtg cttctgcaca cgctcatctt cctggcctgc |
| | agcaacttct ggttcaaatt |
| 961 | cccgcgcacc agctcgaagc tggagcactt tgtgtctatc |
| | ctgctgaagt gcttcgactc |
| 1021 | gccctggacc acgagggccc tgtcggagac agtggtggag |
| | gagagcgacc ccaagccggc |
| 1081 | cttcagcaag atgaatgggt ccatggacaa aaagtcatcg |
| | accgtcagtg aggacgtgga |
| 1141 | ggccaccgtg cccatgctgc agcggaccaa gtcacggatc |
| | gagcagggta tcgtggaccg |
| 1201 | ctcagagacg ggcgtgctgg acaagaagga gggggagcaa |
| | gccaaggcgc tgtttgagaa |
| 1261 | ggtgaagaag ttccggaccc atgtggagga gggggacatt |
| | gtgtaccgcc tctacatgcg |
| 1321 | gcagaccatc atcaaggtga tcaagttcat cctcatcatc |
| | tgctacaccg tctactacgt |
| 1381 | gcacaacatc aagttcgacg tggactgcac cgtggacatt |
| | gagagcctga cgggctaccg |
| 1441 | cacctaccgc tgtgcccacc ccctggccac actcttcaag |
| | atcctggcgt ccttctacat |
| 1501 | cagcctagtc atcttctacg gcctcatctg catgtataca |
| | ctgtggtgga tgctacggcg |
| 1561 | ctccctcaag aagtactcgt ttgagtcgat ccgtgaggag |
| | agcagctaca gcgacatccc |
| 1621 | cgacgtcaag aacgacttcg ccttcatgct gcacctcatt |
| | gaccaatacg acccgctcta |
| 1681 | ctccaagcgc ttcgccgtct tcctgtcgga ggtgagtgag |
| | aacaagctgc ggcagctgaa |
| 1741 | cctcaacaac gagtggacgc tggacaagct ccggcagcgg |
| | ctcaccaaga acgcgcagga |
| 1801 | caagctggag ctgcacctgt tcatgctcag tggcatccct |
| | gacactgtgt ttgacctggt |
| 1861 | ggagctggag gtcctcaagc tggagctgat ccccgacgtg |
| | accatcccgc ccagcattgc |
| 1921 | ccagctcacg ggcctcaagg agctgtggct ctaccacaca |
| | gcggccaaga ttgaagcgcc |
| 1981 | cgcgctggcc ttcctgcgcg agaacctgcg ggcgctgcac |
| | atcaagttca ccgacatcaa |
| 2041 | ggagatcccg ctgtggatct atagcctgaa gacactggag |
| | gagctgcacc tgacgggcaa |
| 2101 | cctgagcgcg gagaacaacc gctacatcgt catcgacggg |
| | ctgcgggagc tcaaacgcct |
| 2161 | caaggtgctg cggctcaaga gcaacctaag caagctgcca |
| | caggtggtca cagatgtggg |
| 2221 | cgtgcacctg cagaagctgt ccatcaacaa tgagggcacc |
| | aagctcatcg tcctcaacag |
| 2281 | cctcaagaag atggcgaacc tgactgagct ggagctgatc |
| | cgctgtgacc tggagcgcat |
| 2341 | cccccactcc atcttcagcc tccacaacct gcaggagatt |
| | gacctcaagg acaacaacct |
| 2401 | caagaccatc gaggagatca tcagcttcca gcacctgcac |
| | cgcctcacct gccttaagct |
| 2461 | gtggtacaac cacatcgcct acatccccat ccagatcggc |
| | aacctcacca acctggagcg |
| 2521 | cctctacctg aaccgcaaca agatcgagaa gatccccacc |
| | cagctcttct actgccgcaa |
| 2581 | gctgcgctac ctggacctca gccacaacaa cctgaccttc |
| | ctccctgccg acatcggcct |
| 2641 | cctgcagaac ctccagaacc tagccatcac ggccaaccgg |
| | atcgagacgc tccctccgga |
| 2701 | gctcttccag tgccggaagc tgcgggccct gcacctgggc |
| | aacaacgtgc tgcagtcact |
| 2761 | gccctccagg gtgggcgagc tgaccaacct gacgcagatc |
| | gagctgcggg gcaaccggct |
| 2821 | ggagtgcctg cctgtggagc tgggcgagtg cccactgctc |
| | aagcgcagcg gcttggtggt |
| 2881 | ggaggaggac ctgttcaaca cactgccacc cgaggtgaag |
| | gagcggctgt ggagggctga |
| 2941 | caaggagcag gcctga |

In one embodiment, LRRC8A is depleted from the cell's genome using any genome editing system including, but not limited to, zinc finger nucleases, TALENS, meganucleases, and CRISPR/Cas systems. In one embodiment, the genomic editing system used to incorporate the nucleic acid encoding one or more guide RNAs into the cell's genome is not a CRISPR/Cas system; this can prevent undesirable cell death in cells that retain a small amount of Cas enzyme/protein. It is also contemplated herein that either the Cas enzyme or the sgRNAs are each expressed under the control of a different inducible promoter, thereby allowing temporal expression of each to prevent such interference.

When a nucleic acid encoding one or more sgRNAs and a nucleic acid encoding an RNA-guided endonuclease each need to be administered, the use of an adenovirus associated vector (AAV) is specifically contemplated. Other vectors for simultaneously delivering nucleic acids to both components of the genome editing/fragmentation system (e.g., sgRNAs, RNA-guided endonuclease) include lentiviral vectors, such as Epstein Barr, Human immunodeficiency virus (HIV), and hepatitis B virus (HBV). Each of the components of the RNA-guided genome editing system (e.g., sgRNA and endonuclease) can be delivered in a separate vector as known in the art or as described herein.

In one embodiment, the agent inhibits LRRC8A by RNA inhibition. Inhibitors of the expression of a given gene can be an inhibitory nucleic acid. In some embodiments of any of the aspects, the inhibitory nucleic acid is an inhibitory RNA (iRNA). The RNAi can be single stranded or double stranded.

The iRNA can be siRNA, shRNA, endogenous microRNA (miRNA), or artificial miRNA. In one embodiment, an iRNA as described herein effects inhibition of the expression and/or activity of a target, e.g. LRRC8A. In some embodiments of any of the aspects, the agent is siRNA that inhibits LRRC8A. In some embodiments of any of the aspects, the agent is shRNA that inhibits LRRC8A.

One skilled in the art would be able to design siRNA, shRNA, or miRNA to target LRRC8A, e.g., using publically available design tools. siRNA, shRNA, or miRNA is commonly made using companies such as Dharmacon (Layfayette, Colo.) or Sigma Aldrich (St. Louis, Mo.).

In some embodiments of any of the aspects, the iRNA can be a dsRNA. A dsRNA includes two RNA strands that are sufficiently complementary to hybridize to form a duplex structure under conditions in which the dsRNA will be used. One strand of a dsRNA (the antisense strand) includes a region of complementarity that is substantially complementary, and generally fully complementary, to a target sequence. The target sequence can be derived from the sequence of an mRNA formed during the expression of the target. The other strand (the sense strand) includes a region that is complementary to the antisense strand, such that the two strands hybridize and form a duplex structure when combined under suitable conditions

The RNA of an iRNA can be chemically modified to enhance stability or other beneficial characteristics. The nucleic acids featured in the invention may be synthesized and/or modified by methods well established in the art, such as those described in “Current protocols in nucleic acid chemistry,” Beaucage, S. L. et al. (Edrs.), John Wiley & Sons, Inc., New York, N.Y., USA, which is hereby incorporated herein by reference.

In one embodiment, the agent is miRNA that inhibits LRRC8A. microRNAs are small non-coding RNAs with an average length of 22 nucleotides. These molecules act by binding to complementary sequences within mRNA molecules, usually in the 3′ untranslated (3′UTR) region, thereby promoting target mRNA degradation or inhibited mRNA translation. The interaction between microRNA and mRNAs is mediated by what is known as the “seed sequence”, a 6-8-nucleotide region of the microRNA that directs sequence-specific binding to the mRNA through imperfect Watson-Crick base pairing. More than 900 microRNAs are known to be expressed in mammals. Many of these can be grouped into families on the basis of their seed sequence, thereby identifying a “cluster” of similar microRNAs. A miRNA can be expressed in a cell, e.g., as naked DNA. A miRNA can be encoded by a nucleic acid that is expressed in the cell, e.g., as naked DNA or can be encoded by a nucleic acid that is contained within a vector.

The agent may result in gene silencing of the target gene (e.g., LRRC8A), such as with an RNAi molecule (e.g. siRNA or miRNA). This entails a decrease in the mRNA level in a cell for a target by at least about 5%, about 10%, about 20%, about 30%, about 40%, about 50%, about 60%, about 70%, about 80%, about 90%, about 95%, about 99%, about 100% of the mRNA level found in the cell without the presence of the agent. In one preferred embodiment, the mRNA levels are decreased by at least about 70%, about 80%, about 90%, about 95%, about 99%, about 100%. One skilled in the art will be able to readily assess whether the siRNA, shRNA, or miRNA effective target e.g., LRRC8A, for its downregulation, for example by transfecting the siRNA, shRNA, or miRNA into cells and detecting the levels of a gene or gene product (e.g., LRRC8A) found within the cell via PCR-based assay or western-blotting, respectively.

The agent may be contained in and thus further include a vector. Many such vectors useful for transferring exogenous genes into target mammalian cells are available. The vectors may be episomal, e.g. plasmids, virus-derived vectors such cytomegalovirus, adenovirus, etc., or may be integrated into the target cell genome, through homologous recombination or random integration, e.g. retrovirus-derived vectors such as MMLV, HIV-1, ALV, etc. In some embodiments, combinations of retroviruses and an appropriate packaging cell line may also find use, where the capsid proteins will be functional for infecting the target cells. Usually, the cells and virus will be incubated for at least about 24 hours in the culture medium. The cells are then allowed to grow in the culture medium for short intervals in some applications, e.g. 24-73 hours, or for at least two weeks, and may be allowed to grow for five weeks or more, before analysis. Commonly used retroviral vectors are “defective”, i.e. unable to produce viral proteins required for productive infection. Replication of the vector requires growth in the packaging cell line.

The term “vector”, as used herein, refers to a nucleic acid construct designed for delivery to a host cell or for transfer between different host cells. As used herein, a vector can be viral or non-viral. The term “vector” encompasses any genetic element that is capable of replication when associated with the proper control elements and that can transfer gene sequences to cells. A vector can include, but is not limited to, a cloning vector, an expression vector, a plasmid, phage, transposon, cosmid, artificial chromosome, virus, virion, etc.

As used herein, the term “expression vector” refers to a vector that directs expression of an RNA or polypeptide (e.g., an LRRC8A inhibitor) from nucleic acid sequences contained therein linked to transcriptional regulatory sequences on the vector. The sequences expressed will often, but not necessarily, be heterologous to the cell. An expression vector may comprise additional elements, for example, the expression vector may have two replication systems, thus allowing it to be maintained in two organisms, for example in human cells for expression and in a prokaryotic host for cloning and amplification. The term “expression” refers to the cellular processes involved in producing RNA and proteins and as appropriate, secreting proteins, including where applicable, but not limited to, for example, transcription, transcript processing, translation and protein folding, modification and processing. “Expression products” include RNA transcribed from a gene, and polypeptides obtained by translation of mRNA transcribed from a gene. The term “gene” means the nucleic acid sequence which is transcribed (DNA) to RNA in vitro or in vivo when operably linked to appropriate regulatory sequences. The gene may or may not include regions preceding and following the coding region, e.g. 5′ untranslated (5′UTR) or “leader” sequences and 3′ UTR or “trailer” sequences, as well as intervening sequences (introns) between individual coding segments (exons).

Integrating vectors have their delivered RNA/DNA permanently incorporated into the host cell chromosomes. Non-integrating vectors remain episomal which means the nucleic acid contained therein is never integrated into the host cell chromosomes. Examples of integrating vectors include retroviral vectors, lentiviral vectors, hybrid adenoviral vectors, and herpes simplex viral vector.

One example of a non-integrative vector is a non-integrative viral vector. Non-integrative viral vectors eliminate the risks posed by integrative retroviruses, as they do not incorporate their genome into the host DNA. One example is the Epstein Barr oriP/Nuclear Antigen-1 (“EBNA1”) vector, which is capable of limited self-replication and known to function in mammalian cells. As containing two elements from Epstein-Barr virus, oriP and EBNA1, binding of the EBNA1 protein to the virus replicon region oriP maintains a relatively long-term episomal presence of plasmids in mammalian cells. This particular feature of the oriP/EBNA1 vector makes it ideal for generation of integration-free iPSCs. Another non-integrative viral vector is adenoviral vector and the adeno-associated viral (AAV) vector.

Another non-integrative viral vector is RNA Sendai viral vector, which can produce protein without entering the nucleus of an infected cell. The F-deficient Sendai virus vector remains in the cytoplasm of infected cells for a few passages, but is diluted out quickly and completely lost after several passages (e.g., 10 passages).

Another example of a non-integrative vector is a minicircle vector. Minicircle vectors are circularized vectors in which the plasmid backbone has been released leaving only the eukaryotic promoter and cDNA(s) that are to be expressed.

As used herein, the term “viral vector” refers to a nucleic acid vector construct that includes at least one element of viral origin and has the capacity to be packaged into a viral vector particle. The viral vector can contain a nucleic acid encoding a polypeptide as described herein in place of non-essential viral genes. The vector and/or particle may be utilized for the purpose of transferring nucleic acids into cells either in vitro or in vivo. Numerous forms of viral vectors are known in the art.

### Compositions

One aspect provided herein is a composition comprising any of the agents that inhibit LRRC8A, as described herein. For example, the agent is a polyclonal antibody that targets LRRC8A via an antigen having a sequence of SEQ ID NO: 3 or 4.

In one embodiment, the composition further comprises a pharmaceutically acceptable carrier. As used herein, the term “pharmaceutically acceptable”, and grammatical variations thereof, as they refer to compositions, carriers, diluents and reagents, are used interchangeably and represent that the materials are capable of administration to or upon a mammal without the production of undesirable physiological effects such as nausea, dizziness, gastric upset and the like. Each carrier must also be “acceptable” in the sense of being compatible with the other ingredients of the formulation. A pharmaceutically acceptable carrier will not promote the raising of an immune response to an agent with which it is admixed, unless so desired. The preparation of a pharmacological composition that contains active ingredients dissolved or dispersed therein is well understood in the art and need not be limited based on formulation. The pharmaceutical formulation contains a compound of the invention in combination with one or more pharmaceutically acceptable ingredients. The carrier can be in the form of a solid, semi-solid or liquid diluent, cream or a capsule. Typically, such compositions are prepared as injectable either as liquid solutions or suspensions, however, solid forms suitable for solution, or suspensions, in liquid prior to use can also be prepared. The preparation can also be emulsified or presented as a liposome composition. The active ingredient can be mixed with excipients which are pharmaceutically acceptable and compatible with the active ingredient and in amounts suitable for use in the therapeutic methods described herein. Suitable excipients are, for example, water, saline, dextrose, glycerol, ethanol or the like and combinations thereof. In addition, if desired, the composition can contain minor amounts of auxiliary substances such as wetting or emulsifying agents, pH buffering agents and the like which enhance the effectiveness of the active ingredient. The therapeutic composition of the present invention can include pharmaceutically acceptable salts of the components therein. Pharmaceutically acceptable salts include the acid addition salts (formed with the free amino groups of the polypeptide) that are formed with inorganic acids such as, for example, hydrochloric or phosphoric acids, or such organic acids as acetic, tartaric, mandelic and the like. Salts formed with the free carboxyl groups can also be derived from inorganic bases such as, for example, sodium, potassium, ammonium, calcium or ferric hydroxides, and such organic bases as isopropylamine, trimethylamine, 2-ethylamino ethanol, histidine, procaine and the like. Physiologically tolerable carriers are well known in the art. Exemplary liquid carriers are sterile aqueous solutions that contain no materials in addition to the active ingredients and water, or contain a buffer such as sodium phosphate at physiological pH value, physiological saline or both, such as phosphate-buffered saline. Still further, aqueous carriers can contain more than one buffer salt, as well as salts such as sodium and potassium chlorides, dextrose, polyethylene glycol and other solutes. Liquid compositions can also contain liquid phases in addition to and to the exclusion of water. Exemplary of such additional liquid phases are glycerin, vegetable oils such as cottonseed oil, and water-oil emulsions. The amount of an active agent used in the invention that will be effective in the treatment of a particular disorder or condition will depend on the nature of the disorder or condition, and can be determined by standard clinical techniques. The phrase “pharmaceutically acceptable carrier or diluent” means a pharmaceutically acceptable material, composition or vehicle, such as a liquid or solid filler, diluent, excipient, solvent or encapsulating material, involved in carrying or transporting the subject agents from one organ, or portion of the body, to another organ, or portion of the body.

In one embodiment, the composition further comprises at least a second therapeutic used in the treatment of a given autoimmune disease, e.g., GVHD or diabetes.

Exemplary treatments for GVHD include but are not limited to, Immunosuppressive drugs, e.g., Cyclosporine (Neoral, Sandimmune, Gengraf, and Restasis), Tacrolimus (Prograf, Protopic, Astagraf XL, and Envarsus XR), Methotrexate (Trexall, Rasuvo, Rheumatrex, and Otrexup (PF)), Sirolimus (Rapamune), Mycophenolic acid (Myfortic and CellCept), Rituximab (Rituxan), etanercept (Enbrel), pentostatin (Nipent), ruxolitinib (Jakafi); Chemotherapies, e.g., Methotrexate (Trexall, Rasuvo, Rheumatrex, and Otrexup (PF)), antithymocyte globulin (Atgam, Thymoglobulin); Steroids, e.g., Prednisone (Deltasone, Rayos, and Prednisone Intensol), Methylprednisolone (Medrol, Solu-Medrol, and Depo-Medrol), budesonide (Entocort EC, Uceris); Antifungal, e.g., Posaconazole (Noxafil); Antiviral drugs, e.g., Acyclovir (Zovirax and Sitavig), Valacyclovir (Valtrex); and Antibiotics, e.g., Sulfamethoxazole/Trimethoprim (Bactrim, Sulfatrim, and Bactrim DS); Protease inhibitors, e.g. alphas-proteinase inhibitor (Zemaira); extracorporeal photopheresis; monoclonal antibodies (daclizumab (Zinbryta), basiliximab (Simulect)), Brentuximab vedotin (Adcetris), Alemtuzumab (Campath, Lemtrada), Tocilizumab (Actemra); infusion of mesenchymal stromal cells.

Exemplary treatments for diabetes include but are not limited to, Insulin, e.g., Insulin glulisine (Apidra and Apidra SoloStar), Insulin detemir (Levemir and Levemir FlexTouch), Insulin aspart (NovoLog, Novolog Flexpen, and Novolog PenFill), Insulin lispro (Humalog and Humalog KwikPen), Insulin, Insulin glargine (Lantus, Lantus Solostar, and Toujeo SoloStar); Dietary supplement, e.g., glucose tablets; and Hormones, e.g., Glucagon (GlucaGen and Glucagon Emergency Kit (human)), antidiabetic agents (Metformin (D-Care DM2, Fortamet, Glucophage, Glucophage XR, Glumetza, Riomet), glucagon-like peptide-1 (GLP-1) receptor agonist (liraglutide (Saxenda; Victoza) or semaglutide (Ozempic) or sodium-glucose co-transporter 2 (SGLT2) inhibitor: empagliflozin (Jardiance), canagliflozin (Invokana); sulfonylureas: glipizide (GlipiZIDE XL, Glucotrol, Glucotrol XL); Meglitinide Analogs: repaglinide (Prandin); Thiazolidinedione: pioglitazone (Actos); dipeptidyl peptidase-4 [DPP-4] inhibitors: Sitagliptin (Januvia), Saxagliptin (Onglyza), Linagliptin (Tradjenta), Alogliptin (Nesina).

In one embodiment, the composition further comprises any treatment or therapeutic described herein, for example, insulin, Abatacept (Orencia®) or belatacept (Nulojix®).

Compositions described herein can be formulated for any route of administration described herein below. Methods for formulating a composition for a desired administration are further discussed below.

One aspect of the invention is the use of any composition described herein for the treatment of GVHD.

Another aspect of the invention is the use of any composition described herein for the treatment of diabetes.

### Administration

In some embodiments, the methods described herein relate to treating a subject having or diagnosed as having an autoimmune disease, for example GVHD or diabetes, comprising administering an agent that inhibits LRRC8A as described herein. Subjects having an GVHD or diabetes can be identified by a physician using current methods (i.e. assays) of diagnosing a condition. Symptoms and/or complications of GVHD or diabetes, which characterize these disease and aid in diagnosis are well known in the art and include but are not limited to those symptoms described herein above. Tests that may aid in a diagnosis of, e.g. GVHD or diabetes are known in the art and described herein above. A family history of, e.g., GVHD or diabetes, will also aid in determining if a subject is likely to have the condition or in making a diagnosis of GVHD or diabetes.

The agents or compositions comprising the agent described herein (e.g., an agent that inhibits LRRC8A) can be administered to a subject having or diagnosed as having GVHD or diabetes. In some embodiments, the methods described herein comprise administering an effective amount of an agent to a subject in order to alleviate at least one symptom of, e.g., GVHD or diabetes. As used herein, “alleviating at least one symptom of GVHD or diabetes” is ameliorating any condition or symptom associated with, e.g., GVHD or diabetes (symptoms associated with GVHD and diabetes are described herein above). As compared with an equivalent untreated control, such reduction is by at least 5%, 10%, 20%, 40%, 50%, 60%, 80%, 90%, 95%, 99% or more as measured by any standard technique. A variety of means for administering the agents and compositions described herein to subjects are known to those of skill in the art. In one embodiment, the agent is administered systemically or locally (e.g., to a particular organ or tissue type). In one embodiment, the agent is administered intravenously. In one embodiment, the agent is administered continuously, in intervals, or sporadically. The route of administration of the agent will be optimized for the type of agent being delivered (e.g., an antibody, a small molecule, an RNAi), and can be determined by a skilled practitioner. In one embodiment, the agent, or compositions comprising an agent are administered through inhalation, for example, for delivery to the lungs. Thus, in one embodiment, a composition comprising an agent described herein is formulated for aerosol delivery.

The term “effective amount” as used herein refers to the amount of an agent (e.g., an agent that inhibits LRCCA), or a composition thereof, can be administered to a subject having or diagnosed as having an autoimmune disease needed to alleviate at least one or more symptom of, e.g., GVHD or diabetes. The term “therapeutically effective amount” therefore refers to an amount of an agent that is sufficient to provide, e.g., a particular anti-autoimmune disease effect when administered to a typical subject. An effective amount as used herein, in various contexts, would also include an amount of an agent sufficient to delay the development of a symptom of, e.g., GVHD or diabetes, alter the course of a symptom of, e.g., GVHD or diabetes, or reverse a symptom of, e.g., GVHD or diabetes. Symptoms associated with GVHD and diabetes are described herein above. Thus, it is not generally practicable to specify an exact “effective amount”. However, for any given case, an appropriate “effective amount” can be determined by one of ordinary skill in the art using only routine experimentation.

In one embodiment, the agent, or composition thereof is administered continuously (e.g., at constant levels over a period of time). Continuous administration of an agent can be achieved, e.g., by epidermal patches, continuous release formulations, or on-body injectors.

In one embodiment, the agent described herein can be administered at least once a day, a week, every 3 weeks, a month, every 2 months, every 3 months, every 4 months, every 5 months, every 6 months, every 7 months, every 8 months, every 9 months, every 10 months, every 11 months, a year, or more. The agent can be administered for a duration to be determined by a skilled practitioner, for example, for at least 1 week, at least 2 weeks, at least 1 month, at least 2 months, at least 1 year, or more. Alternatively, the administration of the agent described herein can be continuous, for example, for the duration of the subject's disease state.

Effective amounts, toxicity, and therapeutic efficacy can be evaluated by standard pharmaceutical procedures in cell cultures or experimental animals. The dosage can vary depending upon the dosage form employed and the route of administration utilized. The dose ratio between toxic and therapeutic effects is the therapeutic index and can be expressed as the ratio LD50/ED50. Compositions and methods that exhibit large therapeutic indices are preferred. A therapeutically effective dose can be estimated initially from cell culture assays. Also, a dose can be formulated in animal models to achieve a circulating plasma concentration range that includes the IC50 (i.e., the concentration of the agent, which achieves a half-maximal inhibition of symptoms) as determined in cell culture, or in an appropriate animal model. Levels in plasma can be measured, for example, by high performance liquid chromatography. The effects of any particular dosage can be monitored by a suitable bioassay, e.g., measuring neurological function, or blood work, among others. The dosage can be determined by a physician and adjusted, as necessary, to suit observed effects of the treatment.

### Dosage

“Unit dosage form” as the term is used herein refers to a dosage for suitable one administration. By way of example a unit dosage form can be an amount of therapeutic disposed in a delivery device, e.g., a syringe or intravenous drip bag. In one embodiment, a unit dosage form is administered in a single administration. In another, embodiment more than one unit dosage form can be administered simultaneously.

The dosage of the agent as described herein can be determined by a physician and adjusted, as necessary, to suit observed effects of the treatment. With respect to duration and frequency of treatment, it is typical for skilled clinicians to monitor subjects in order to determine when the treatment is providing therapeutic benefit, and to determine whether to administer further cells, discontinue treatment, resume treatment, or make other alterations to the treatment regimen. The dosage should not be so large as to cause adverse side effects, such as cytokine release syndrome. Generally, the dosage will vary with the age, condition, and sex of the patient and can be determined by one of skill in the art. The dosage can also be adjusted by the individual physician in the event of any complication.

### Combinational Therapy

In one embodiment, the agent described herein is used as a monotherapy. In one embodiment, the agents described herein can be used in combination with other known agents and therapies for an autoimmune disease (e.g., GVHD or diabetes). Administered “in combination,” as used herein, means that two (or more) different treatments are delivered to the subject during the course of the subject's affliction with the disorder, e.g., the two or more treatments are delivered after the subject has been diagnosed with the disorder or disease (for example, GVHD or diabetes) and before the disorder has been cured or eliminated or treatment has ceased for other reasons. In some embodiments, the delivery of one treatment is still occurring when the delivery of the second begins, so that there is overlap in terms of administration. This is sometimes referred to herein as “simultaneous” or “concurrent delivery.” In other embodiments, the delivery of one treatment ends before the delivery of the other treatment begins. In some embodiments of either case, the treatment is more effective because of combined administration. For example, the second treatment is more effective, e.g., an equivalent effect is seen with less of the second treatment, or the second treatment reduces symptoms to a greater extent, than would be seen if the second treatment were administered in the absence of the first treatment, or the analogous situation is seen with the first treatment. In some embodiments, delivery is such that the reduction in a symptom, or other parameter related to the disorder is greater than what would be observed with one treatment delivered in the absence of the other. The effect of the two treatments can be partially additive, wholly additive, or greater than additive. The delivery can be such that an effect of the first treatment delivered is still detectable when the second is delivered. The agents described herein and the at least one additional therapy can be administered simultaneously, in the same or in separate compositions, or sequentially. For sequential administration, the agent described herein can be administered first, and the additional agent can be administered second, or the order of administration can be reversed. The agent and/or other therapeutic agents, procedures or modalities can be administered during periods of active disorder, or during a period of remission or less active disease. The agent can be administered before another treatment, concurrently with the treatment, post-treatment, or during remission of the disorder.

Exemplary therapeutics used to treat GVHD and diabetes are described herein above.

When administered in combination, the agent and the additional agent (e.g., second or third agent), or all, can be administered in an amount or dose that is higher, lower or the same as the amount or dosage of each agent used individually, e.g., as a monotherapy. In certain embodiments, the administered amount or dosage of the agent, the additional agent (e.g., second or third agent), or all, is lower (e.g., at least 20%, at least 30%, at least 40%, or at least 50%) than the amount or dosage of each agent used individually. In other embodiments, the amount or dosage of agent, the additional agent (e.g., second or third agent), or all, that results in a desired effect (e.g., treatment of GVHD or diabetes) is lower (e.g., at least 20%, at least 30%, at least 40%, or at least 50% lower) than the amount or dosage of each agent individually required to achieve the same therapeutic effect.

### Parenteral Dosage Forms

Parenteral dosage forms of an agents described herein can be administered to a subject by various routes, including, but not limited to, subcutaneous, intravenous (including bolus injection), intramuscular, and intraarterial. Since administration of parenteral dosage forms typically bypasses the patient's natural defenses against contaminants, parenteral dosage forms are preferably sterile or capable of being sterilized prior to administration to a patient. Examples of parenteral dosage forms include, but are not limited to, solutions ready for injection, dry products ready to be dissolved or suspended in a pharmaceutically acceptable vehicle for injection, suspensions ready for injection, controlled-release parenteral dosage forms, and emulsions.

Suitable vehicles that can be used to provide parenteral dosage forms of the disclosure are well known to those skilled in the art. Examples include, without limitation: sterile water; water for injection USP; saline solution; glucose solution; aqueous vehicles such as but not limited to, sodium chloride injection, Ringer's injection, dextrose Injection, dextrose and sodium chloride injection, and lactated Ringer's injection; water-miscible vehicles such as, but not limited to, ethyl alcohol, polyethylene glycol, and propylene glycol; and non-aqueous vehicles such as, but not limited to, corn oil, cottonseed oil, peanut oil, sesame oil, ethyl oleate, isopropyl myristate, and benzyl benzoate.

### Aerosol Formulations

An agent that inhibits LRRC8A or composition comprising an agent that inhibits LRRC8A can be administered directly to the airways of a subject in the form of an aerosol or by nebulization. For use as aerosols, an agent that inhibits LRRC8A in solution or suspension may be packaged in a pressurized aerosol container together with suitable propellants, for example, hydrocarbon propellants like propane, butane, or isobutane with conventional adjuvants. An agent that inhibits LRRC8A can also be administered in a non-pressurized form such as in a nebulizer or atomizer. Aerosol formulations can be used administer an agent directly to the airway of a subject, e.g., for treatment of any component of an airway. For example, an aerosol formulation comprising an anti-LRRC8A agent can be used to treat GVHD following a lung transplant.

The term “nebulization” is well known in the art to include reducing liquid to a fine spray. Preferably, by such nebulization small liquid droplets of uniform size are produced from a larger body of liquid in a controlled manner. Nebulization can be achieved by any suitable means therefore, including by using many nebulizers known and marketed today. For example, an AEROMIST pneumatic nebulizer available from Inhalation Plastic, Inc. of Niles, Ill. When the active ingredients are adapted to be administered, either together or individually, via nebulizer(s) they can be in the form of a nebulized aqueous suspension or solution, with or without a suitable pH or tonicity adjustment, either as a unit dose or multidose device.

As is well known, any suitable gas can be used to apply pressure during the nebulization, with preferred gases to date being those which are chemically inert to a modulator of an agent that inhibits LRRC8A. Exemplary gases including, but are not limited to, nitrogen, argon or helium can be used to high advantage.

In some embodiments, an agent that inhibits LRRC8A can also be administered directly to the airways in the form of a dry powder. For use as a dry powder, a GHK tripeptide can be administered by use of an inhaler. Exemplary inhalers include metered dose inhalers and dry powdered inhalers.

A metered dose inhaler or “MDI” is a pressure resistant canister or container filled with a product such as a pharmaceutical composition dissolved in a liquefied propellant or micronized particles suspended in a liquefied propellant. The propellants which can be used include chlorofluorocarbons, hydrocarbons or hydrofluoroalkanes. Especially preferred propellants are P134a (tetrafluoroethane) and P227 (heptafluoropropane) each of which may be used alone or in combination. They are optionally used in combination with one or more other propellants and/or one or more surfactants and/or one or more other excipients, for example ethanol, a lubricant, an anti-oxidant and/or a stabilizing agent. The correct dosage of the composition is delivered to the patient.

A dry powder inhaler (i.e. Turbuhaler (Astra AB)) is a system operable with a source of pressurized air to produce dry powder particles of a pharmaceutical composition that is compacted into a very small volume.

Dry powder aerosols for inhalation therapy are generally produced with mean diameters primarily in the range of <5 μm. As the diameter of particles exceeds 3 μm, there is increasingly less phagocytosis by macrophages. However, increasing the particle size also has been found to minimize the probability of particles (possessing standard mass density) entering the airways and acini due to excessive deposition in the oropharyngeal or nasal regions.

Suitable powder compositions include, by way of illustration, powdered preparations of an agent that inhibits LRRC8A thoroughly intermixed with lactose, or other inert powders acceptable for intrabronchial administration. The powder compositions can be administered via an aerosol dispenser or encased in a breakable capsule which may be inserted by the patient into a device that punctures the capsule and blows the powder out in a steady stream suitable for inhalation. The compositions can include propellants, surfactants, and co-solvents and may be filled into conventional aerosol containers that are closed by a suitable metering valve.

Aerosols for the delivery to the respiratory tract are known in the art. See for example, Adjei, A. and Garren, J. Pharm. Res., I: 565-569 (1990); Zanen, P. and Lamm, J.-W. J. Int. J. Pharm., 114: 111-115 (1995); Gonda, I. “Aerosols for delivery of therapeutic an diagnostic agents to the respiratory tract,” in Critical Reviews in Therapeutic Drug Carrier Systems, 6:273-313 (1990); Anderson et al., Am. Rev. Respir. Dis., 140: 1317-1324 (1989)) and have potential for the systemic delivery of peptides and proteins as well (Patton and Platz, Advanced Drug Delivery Reviews, 8:179-196 (1992)); Timsina et. al., Int. J. Pharm., 101: 1-13 (1995); and Tansey, I. P., Spray Technol. Market, 4:26-29 (1994); French, D. L., Edwards, D. A. and Niven, R. W., Aerosol Sci., 27: 769-783 (1996); Visser, J., Powder Technology 58: 1-10 (1989)); Rudt, S. and R. H. Muller, J. Controlled Release, 22: 263-272 (1992); Tabata, Y, and Y. Ikada, Biomed. Mater. Res., 22: 837-858 (1988); Wall, D. A., Drug Delivery, 2: 10 1-20 1995); Patton, J. and Platz, R., Adv. Drug Del. Rev., 8: 179-196 (1992); Bryon, P., Adv. Drug. Del. Rev., 5: 107-132 (1990); Patton, J. S., et al., Controlled Release, 28: 15 79-85 (1994); Damms, B. and Bains, W., Nature Biotechnology (1996); Niven, R. W., et al., Pharm. Res., 12(9); 1343-1349 (1995); and Kobayashi, S., et al., Pharm. Res., 13(1): 80-83 (1996), contents of all of which are herein incorporated by reference in their entirety.

### Controlled and Delayed Release Dosage Forms

In some embodiments of the aspects described herein, an agent is administered to a subject by controlled- or delayed-release means. Ideally, the use of an optimally designed controlled-release preparation in medical treatment is characterized by a minimum of drug substance being employed to cure or control the condition in a minimum amount of time. Advantages of controlled-release formulations include: 1) extended activity of the drug; 2) reduced dosage frequency; 3) increased patient compliance; 4) usage of less total drug; 5) reduction in local or systemic side effects; 6) minimization of drug accumulation; 7) reduction in blood level fluctuations; 8) improvement in efficacy of treatment; 9) reduction of potentiation or loss of drug activity; and 10) improvement in speed of control of diseases or conditions. (Kim, Cherng-ju, Controlled Release Dosage Form Design, 2 (Technomic Publishing, Lancaster, Pa.: 2000)). Controlled-release formulations can be used to control a compound of formula (I)'s onset of action, duration of action, plasma levels within the therapeutic window, and peak blood levels. In particular, controlled- or extended-release dosage forms or formulations can be used to ensure that the maximum effectiveness of an agent is achieved while minimizing potential adverse effects and safety concerns, which can occur both from under-dosing a drug (i.e., going below the minimum therapeutic levels) as well as exceeding the toxicity level for the drug.

A variety of known controlled- or extended-release dosage forms, formulations, and devices can be adapted for use with any agent described herein. Examples include, but are not limited to, those described in U.S. Pat. Nos. 3,845,770; 3,916,899; 3,536,809; 3,598,123; 4,008,719; 5,674,533; 5,059,595; 5,591,767; 5,120,548; 5,073,543; 5,639,476; 5,354,556; 5,733,566; and 6,365,185, each of which is incorporated herein by reference in their entireties. These dosage forms can be used to provide slow or controlled-release of one or more active ingredients using, for example, hydroxypropylmethyl cellulose, other polymer matrices, gels, permeable membranes, osmotic systems (such as OROS® (Alza Corporation, Mountain View, Calif. USA)), multilayer coatings, microparticles, liposomes, or microspheres or a combination thereof to provide the desired release profile in varying proportions. Additionally, ion exchange materials can be used to prepare immobilized, adsorbed salt forms of the disclosed compounds and thus effect controlled delivery of the drug. Examples of specific anion exchangers include, but are not limited to, DUOLITE® A568 and DUOLITE® AP143 (Rohm&Haas, Spring House, Pa. USA).

### Efficacy

The efficacy of an agents described herein, e.g., for the treatment of, for example GVHD or diabetes, can be determined by the skilled practitioner. However, a treatment is considered “effective treatment,” as the term is used herein, if one or more of the signs or symptoms of, e.g., GVHD or diabetes, are altered in a beneficial manner, other clinically accepted symptoms are improved, or even ameliorated, or a desired response is induced e.g., by at least 10% following treatment according to the methods described herein. Efficacy can be assessed, for example, by measuring a marker, indicator, symptom, and/or the incidence of a condition treated according to the methods described herein or any other measurable parameter appropriate. Efficacy can also be measured by a failure of an individual to worsen as assessed by hospitalization, or need for medical interventions. Methods of measuring these indicators are known to those of skill in the art and/or are described herein.

Efficacy can be assessed in animal models of a condition described herein, for example, a mouse model or an appropriate animal model of GVHD or diabetes, as the case may be. When using an experimental animal model, efficacy of treatment is evidenced when a statistically significant change in a marker is observed.

Efficacy of an agent that inhibits LRRC8A can additionally be assessed using methods described herein.

All patents, patent applications, and publications identified are expressly incorporated herein by reference for the purpose of describing and disclosing, for example, the methodologies described in such publications that might be used in connection with the present invention. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

The invention provided herein can further be described in any of the numbered paragraphs below.
1. A method for treating or preventing graft versus host disease, the method comprising administering to a subject having, or at risk of developing, graft versus host disease an agent that inhibits LRRC8A.
2. The method of paragraph 1, further comprising, prior to administering, the step of diagnosing a subject as having, or at risk of developing, graft versus host disease.
3. The method of any of the preceding paragraphs, further comprising, prior to administering, the step of receiving the results from an assay that identifies a subject as having, or at risk of developing, graft versus host disease.
4. The method of any of the preceding paragraphs, wherein subject is an organ transplant or hematopoietic stem cell transplant recipient.
5. The method of any of the preceding paragraphs, wherein LRRC8A is inhibited in a T cell or antigen presenting cell.
6. The method of any of the preceding paragraphs, wherein inhibiting results in the blocking of an extracellular portion of LRRC8A on a T cell or an antigen presenting cell.
7. The method of any of the preceding paragraphs, wherein the agent that inhibits LRRC8A is selected from the group consisting of a small molecule, an antibody, a peptide, a genome editing system, an antisense oligonucleotide, and an RNAi.
8. The method of any of the preceding paragraphs, wherein the antibody targets an antigen having a sequence selected from the group consisting of: SEQ ID NO: 3 and SEQ ID NO: 4.
9. The method of any of the preceding paragraphs, wherein the RNAi is a microRNA, an siRNA, or a shRNA.
10. The method of any of the preceding paragraphs, wherein inhibiting LRRC8A is inhibiting the expression level and/or activity of LRRC8A.
11. The method of any of the preceding paragraphs, wherein the expression level and/or activity of LRRC8A is inhibited by at least 50%, at least 60%, at least 70%, at least 80%, at least 90%, or more as compared to an appropriate control.
12. The method of any of the preceding paragraphs, further comprising administering at least a second therapeutic.
13. The method of any of the preceding paragraphs, wherein the second therapeutic is Abatacept (Orencia®) or Belatacept (Nulojix®).
14. A method for treating or preventing graft versus host disease, the method comprising administering to a subject in need thereof an agent that inhibits LRRC8A, wherein the agent is an antibody binds an antigen having a sequence selected from the group consisting of: SEQ ID NO: 3 and SEQ ID NO: 4.
15. The method of any of the preceding paragraphs, further comprising, prior to administering, the step of diagnosing a subject as having, or at risk of developing, graft versus host disease.
16. The method of any of the preceding paragraphs, further comprising, prior to administering, the step of receiving the results from an assay that identifies a subject as having, or at risk of developing, graft versus host disease.
17. The method of any of the preceding paragraphs, wherein subject is an organ transplant or hematopoietic stem cell transplant recipient.
18. The method of any of the preceding paragraphs, further comprising administering at least a second therapeutic.
19. The method of any of the preceding paragraphs, wherein the second therapeutic is Abatacept (Orencia®) or Belatacept (Nulojix®).
20. A method for treating diabetes, the method comprising administering to a subject in need thereof an agent that inhibits LRRC8A.
21. The method of any of the preceding paragraphs, further comprising, prior to administering, the step of diagnosing a subject as having diabetes.
22. The method of any of the preceding paragraphs, further comprising, prior to administering, the step of receiving the results from an assay that identifies a subject as having diabetes.
23. The method of any of the preceding paragraphs, wherein LRRC8A is inhibited in a T cell or antigen presenting cell.
24. The method of any of the preceding paragraphs, wherein inhibiting results in the blocking of an extracellular portion of LRRC8A on a T cell or antigen presenting cell.
25. The method of any of the preceding paragraphs, wherein the agent that inhibits LRRC8A is selected from the group consisting of a small molecule, an antibody, a peptide, a genome editing system, an antisense oligonucleotide, and an RNAi.
26. The method of any of the preceding paragraphs, wherein the antibody targets an antigen having a sequence selected from the group consisting of: SEQ ID NO: 3 and SEQ ID NO: 4.
27. The method of any of the preceding paragraphs, wherein the RNAi is a microRNA, an siRNA, or a shRNA.
28. The method of any of the preceding paragraphs, wherein inhibiting LRRC8A is inhibiting the expression level and/or activity of LRRC8A.
29. The method of any of the preceding paragraphs, wherein the expression level and/or activity of LRRC8A is inhibited by at least 50%, at least 60%, at least 70%, at least 80%, at least 90%, or more as compared to an appropriate control.
30. The method of any of the preceding paragraphs, further comprising administering at least a second therapeutic.
31. The method of any of the preceding paragraphs, wherein the second therapeutic is insulin, Abatacept (Orencia®) or Belatacept (Nulojix®).
32. A method for treating diabetes, the method comprising administering to a subject in need thereof an agent that inhibits LRRC8A, wherein the agent is an antibody binds an antigen having a sequence selected from the group consisting of: SEQ ID NO: 3 and SEQ ID NO: 4.
33. The method of any of the preceding paragraphs, further comprising, prior to administering, the step of diagnosing a subject as having diabetes.
34. The method of any of the preceding paragraphs, further comprising, prior to administering, the step of receiving the results from an assay that identifies a subject as having diabetes.
35. The method of any of the preceding paragraphs, further comprising administering at least a second therapeutic.
36. The method of any of the preceding paragraphs, wherein the second therapeutic is insulin, Abatacept (Orencia®) or Belatacept (Nulojix®).
37. The method of any of the preceding paragraphs, wherein diabetes is type 1 diabetes (T1D) or type 2 diabetes (T2D).
38. A composition comprising an agent that inhibits LRRC8A, wherein the agent is an antibody binds an antigen having a sequence selected from the group consisting of: SEQ ID NO: 3 and SEQ ID NO: 4.
39. The method of any of the preceding paragraphs, further comprising at least a second therapeutic.
40. The method of any of the preceding paragraphs, wherein the second therapeutic is selected from the group consisting of: insulin, Abatacept (Orencia®) or Belatacept (Nulojix®).
41. The method of any of the preceding paragraphs, further comprising a pharmaceutically acceptable carrier or diluent.
42. Use of the composition of any of the preceding paragraphs for the treatment of graft versus host disease.
43. Use of the composition of any of the preceding paragraphs for the treatment of diabetes.
44. The use of any of the preceding paragraphs, wherein diabetes is type 1 diabetes (T1D) or type 2 diabetes (T2D).

### EXAMPLES

### Example 1

Importance of the problem: T cell-driven organ damage is a sequela of primary immunodeficiencies despite differences in upstream molecular defects, as evidenced by the pulmonary disease, enteropathy, and endocrinopathies associated with mutations in PIK3CD, CTLA4, LRBA, and FOXP3, among others.⁷ Minimizing end-organ damage prior to hematopoietic stem cell transplantation improves clinical outcomes.² After transplantation, acute GVHD is another manifestation of T cell-driven organ damage.² The variable clinical responses to inhibitors of T cell activation demonstrate additional unknown mechanisms of T cell activation.⁸

Scientific premise: Work described herein is based on the premise that costimulatory molecules enhance T cell activation by modulating TCR-driven signaling and cellular metabolism.⁹⁻¹¹ The inventors' data show that LRRC8A co-localizes with the TCR and promotes a transcriptional signature characteristic of T cell activation. The inventors' in vitro and in vivo data show that conditional deficiency of LRRC8A impairs the effector and metabolic functions of antigen-experienced CD4⁺ and CD8⁺ T cells. Conditional deletion of LRRC8A on donor CD4⁺ T cells attenuates acute GVHD, demonstrating the relevance of LRRC8A in T cell-driven diseases. An antibody directed against the last 14 C-terminal amino acids of LRRC8A reduces the activation of mouse as well as human T cells.

Biologic variability. Studies are performed on 10-12 week old mice with co-housed littermate controls. Groups of male and female mice are statistically compared.

### Delineate how LRRC8A Promotes the Functions of Effector T Cells.

Preliminary data. We found that LRRC8A is needed for the transition from the second to third double negative (DN2-DN3) stages of thymopoiesis.³ Conditional deletion of LRRC8A at the very late DN4 stage bypasses the requirement for LRRC8A at the DN2-DN3 checkpoint and preserves T cell development in Cd4CreLrrc8a^{f/f} (cKO) mice (FIG. 2).

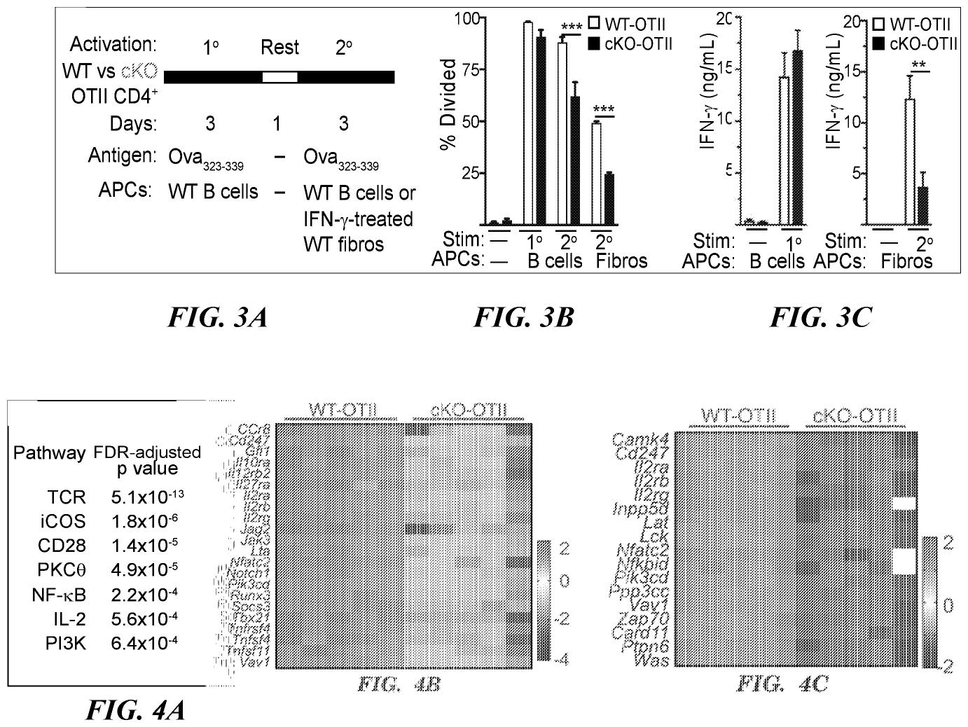

To assess the contribution of LRRC8A to antigen-specific activation, we bred cKO mice on the OTII background to generate LRRC8A-deficient OTII CD4⁺ T cells expressing the transgenic TCR for OVA₃₂₃₋₃₂₉ (cKO-OTII). The primary (1°) response of T cells to a cognate antigen generates antigen-experienced effector and memory T cells capable of responding rapidly to secondary (2°) activation.¹³ Non-hematopoietic APCs, such as fibroblasts, upregulate MEW class II after IFN-γ exposure and activate antigen-experienced T cells, albeit less robustly than hematopoietic APCs.^{14,15} cKO-OTII CD4⁺ T cells had intact proliferation and IFN-γ secretion after 1° activation (FIG. 3A-C). In contrast, antigen-primed cKO-OTII CD4⁺ T cells had impaired proliferation and IFN-γ secretion after 2° stimulation despite intact survival (FIG. 3C-D and data not shown).

After 2° activation, antigen-primed cKO-OTII CD4⁺ T cells had reduced expression of genes downstream of the TCR, CD28, and ICOS (FIG. 4). This is consistent with the inventors' prior study showing that LRRC8A activates Lck-Zap-70-PI3K signaling in thymocytes.³

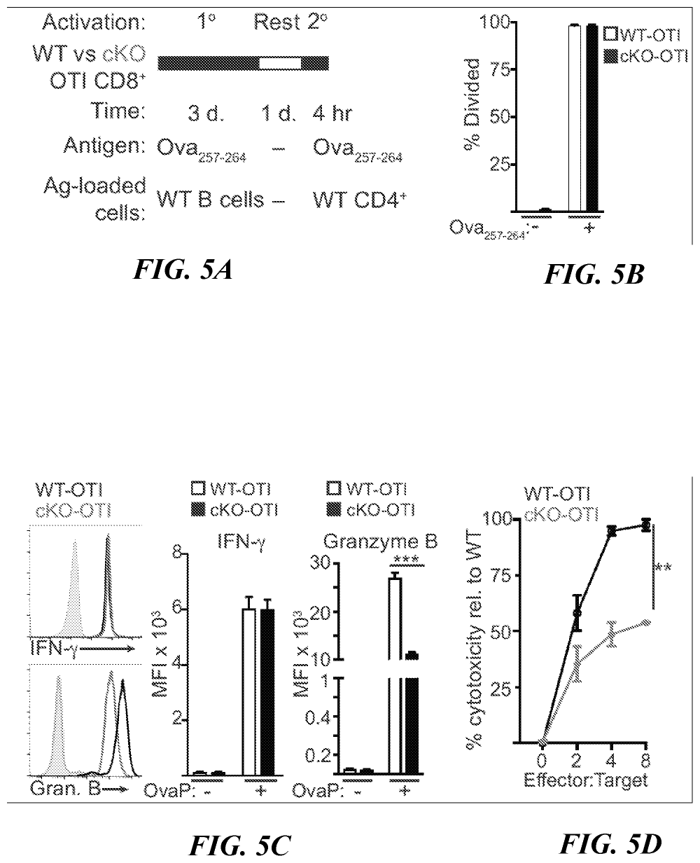

To assess the role of LRRC8A in CD8⁺ T cell function, we generated conditional LRRC8A-deficient CD8⁺ T cells expressing the transgenic TCR specific for OVA₂₅₇₋₂₆₄ peptide (cKO-OTI). After 1° activation, cKO-OTI cells had intact proliferation and expression of IFN-γ, CD25, CD69, and IL-2 (FIG. 5A-C and data not shown). However, cKO-OTI CD8⁺ T cells had reduced expression of granzyme B expression after 1° activation and impaired cytotoxicity after 2° activation (FIG. 5C, D).

Summary. The inventors' data demonstrate the importance for LRRC8A in the activation of antigen-primed CD4⁺ and CD8⁺ T cells. Aim 1 investigates the mechanistic basis for LRRC8A-driven T cell function.

### LRRC8A Complements CD28-Driven Signaling.

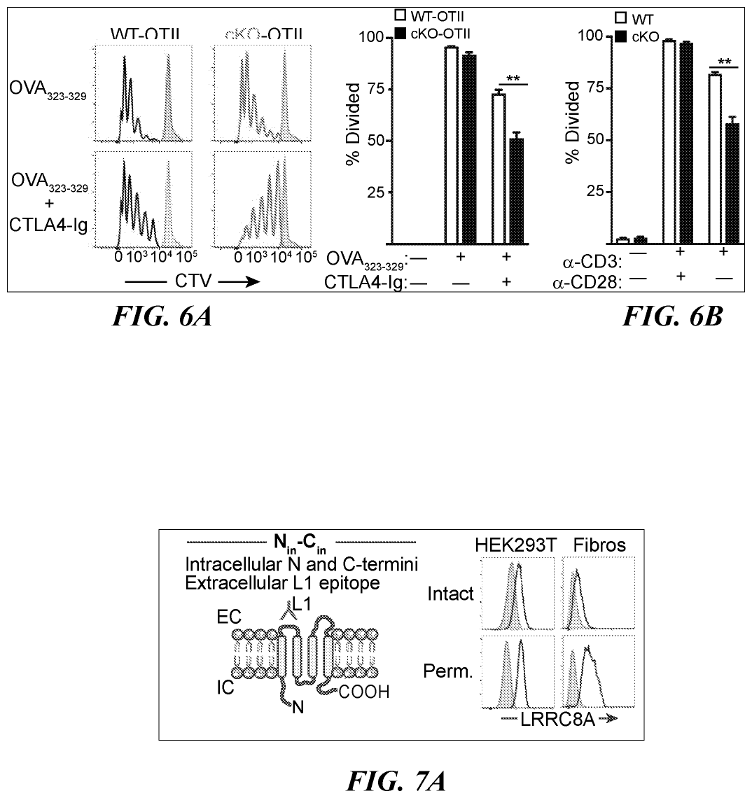

Rationale. The inventors' data show that cKO-OTII CD4⁺ and cKO-OTI CD8⁺ T cells have intact proliferation to 1° stimulation, but impaired 2° activation (FIGS. 3 and 5). Due to the similarities in gene expression downstream of LRRC8A, CD28, and ICOS (FIG. 4C), we show that CD28 and/or ICOS compensates for LRRC8A deficiency during 1° activation. A preliminary experiment demonstrates that CTLA4-Ig inhibits the proliferation of cKO-OTII CD4⁺ T cells more than that of WT-OTII CD4⁺ T cells (FIG. 6A), suggesting at least partial redundancy between CD28 and LRRC8A. Similarly, cKO CD4⁺ T cells proliferated normally to α-CD3+α-CD28, but had impaired proliferation to cross-linked α-CD3 alone. This aim investigates the mechanistic basis for the differential contribution of LRRC8A to the 1° and 2° activation of T cells.

Approach. We can determine whether LRRC8A deficiency±inhibition of CD28 or ICOS affects cell survival, cycling, or exhaustion. WT-OTII or cKO-OTII CD4⁺ T cells can undergo 1° or 2° activation (FIG. 3A), with CTLA4-Ig, the inhibitory anti-ICOS mAb 7E.17G9 (ThermoFisher Scientific), or isotype control. Readouts are described in Table 1. The same can be done for WT-OTI or cKO-OTI CD8⁺ T cells after 1° or 2° activation (FIG. 5A), with the addition of CTLA4-Ig, anti-ICOS mAb, or isotype control.

T cell costimulation is essential for the differentiation of CD4⁺ T effector subsets, as seen in PIDs with defects in ICOS, CD40, CD40L, and OX40.¹⁶⁻¹⁸ To assess the contribution of LRRC8A to CD4⁺ T cell differentiation, we can culture naïve WT vs cKO CD4⁺ T cells under Th1, Th2, and Th17 polarizing conditions.¹⁹

Repeatedly stimulated CD8⁺ T cells undergo transcriptional changes limiting cellular longevity, cycling, and activation.^{20,21} We identified differences in the transcriptional signature of WT-OTII vs cKO-OTII CD4⁺ T cell after 2° activation (FIG. 4), but how LRRC8A influences the evolution of the transcriptional signature in repeatedly stimulated T cells is unknown. We can perform FACS analysis (Table 1) and transcriptome studies of WT-OTII vs cKO-OTII CD4⁺ T cells before and after 4 rounds of stimulation per published methods.^{20,21} The same can be done with WT-OTI vs cKO-OTI CD8⁺ T cells. Transcriptome analysis can be followed by assessment of proteins encoded by the most differentially expressed genes.

### Channel Activity of LRRC8A is not Necessary for T Cell Activation.

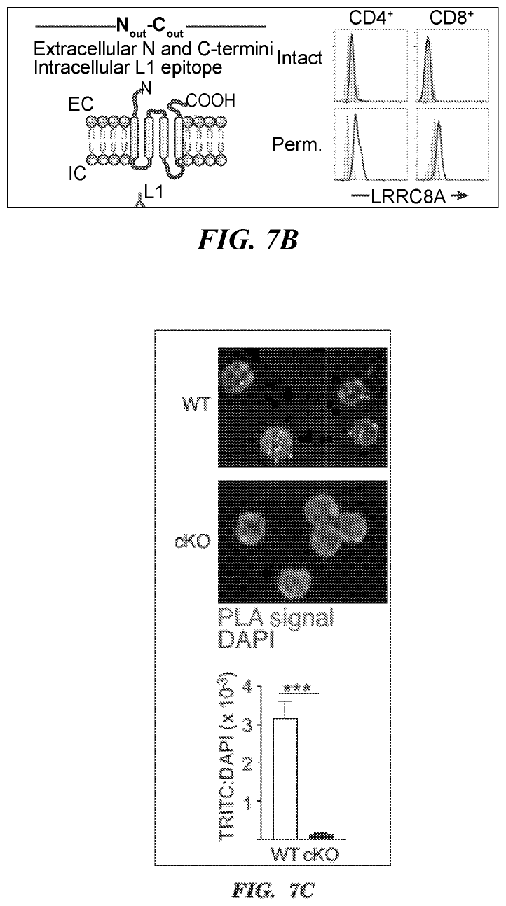

Rationale. Since LRRC8A is important for gene expression downstream of TCR activation (FIG. 4), we used proximal ligation assays to assess potential LRRC8A-TCR colocalization. The proximity (<40 nm) of proteins labeled with oligonucleotide-labeled antibodies enables hybridization and amplification of a circular DNA template using fluorescent oligonucleotides.²⁷ To do this, we addressed apparent discrepancies in the field regarding the orientation of LRRC8A in the plasma membrane. We and others have previously shown that antibodies against the C-terminus of LRRC8A bind to intact T and B cells, indicating an extracellular orientation for the protein's N- and C-termini (Nₒᵤₜ-Cₒᵤₜ).^{3,6,28} In contrast, overexpression studies of epitope-tagged LRRC8A in HEK293T cells and cryo-EM studies of purified protein demonstrate an intracellular orientation for the protein's N- and C-termini (Nᵢₙ-Cᵢₙ). Since membrane transporters, including select anion channels, can have multiple topologies,²⁹ we demonstrated that LRRC8A has a cell-specific orientation. We generated the anti-LRRC8A₈₇₋₁₀₄ antibody (L1) against the loop between the first and second transmembrane regions and demonstrated specificity for LRRC8A.⁶ L1 bound to intact HEK293T cells and murine embryonic fibroblasts (FIG. 7A), indicating an extracellular L1 epitope and an intracellular orientation for the LRRC8A N- and C-termini (Nᵢₙ-Cᵢₙ). L1 binding increased in permeabilized stromal cells (FIG. 7A), suggesting the presence of intracellular LRRC8A (addressed in Aim 2). In contrast, L1 had no binding to the surface of intact WT T cells. L1 bound to permeabilized T cells, consistent with an intracellular L1 epitope and an exclusively Nₒᵤₜ-Cₒᵤₜ orientation for LRRC8A on T cells (FIG. 7B).

Having confirmed the presence of an extracellular C-terminus for LRRC8A on T cells, we then performed in situ PLA on resting CD3⁺ T cells using antibodies against the extracellular portion of CD3ε and the extracellular C-terminus of LRRC8A. We found co-localization of LRRC8A and CD3ε in WT T cells (FIG. 7C).

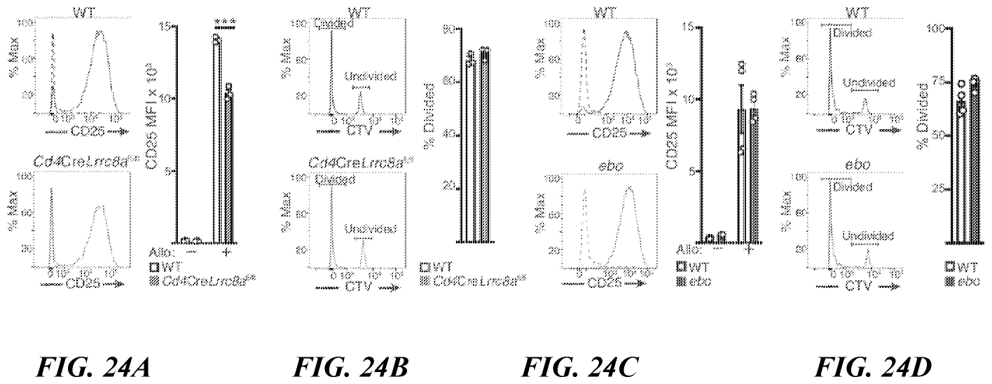

LRRC8A is the essential pore-forming subunit of the volume-regulated anion channel (VRAC) activated by hypotonic stress.^{4,5} There are no known inhibitors specific for VRAC and it is unknown if T cell activation triggers VRAC activity under isotonic conditions.^{30,31} However, we showed that ebo mice express a mutant form of LRRC8A lacking 15 C-terminal leucine-rich repeats as well as channel activity due to a 2 base pair deletion in Lrrc8a.⁶ Mutant LRRC8A is expressed on ebo cells at levels similar to WT LRRC8A on WT cells and ebo mice have normal thymocyte development, indicating that VRAC activity is dispensable for T cell development.⁶ Ebo T cells have preserved proliferation to 1° activation.⁶ We have showed that ebo mice have an intact mixed lymphocyte reaction (FIGS. 24C and 24D), an assay that models GVHD in vitro, demonstrating that the channel activity of LRRC8A is not necessary for the 1° or 2° response of T cells.

Approach. We can assess readouts for T cell activation (Table 1) in WT-DO11.10 vs ebo-DO11.10 CD4⁺ T cells at rest and after 1° and 2° activation by WT Balb/c B cells presenting OVA₃₂₃₋₃₃₉. As Ca²⁺ flux is central to signaling at active immune synapses,^{32,33} we can assess the contribution of LRRC8A to Ca′ flux in activated WT, cKO, and ebo T cells. We can load CD4⁺ or CD8⁺ T cells from each genotype with the ratiometric Ca′ indicator Fura-2, followed by activation with anti-CD3 crosslinking+CD28 or phorbol 12-myristate 13-acetate and ionomycin as a positive control. Ca²⁺ flux can be assessed by flow cytometry for 10 minutes after stimulation.

To determine if LRRC8A colocalizes to active immune synapses, we can assess the distribution of LRRC8A relative to phospho-Zap70 and phospho-Vav utilizing laser scanning confocal microscopy.³⁴ For 1° activation studies, LPS-matured bone marrow-derived dendritic cells (BMOCs) can be loaded with OVA₃₂₃₋₃₃₉ and co-cultured with WT vs ebo-DO11.10 CD4⁺ T cells at a 1:1 ratio for 0, 10, 20, and 30 minutes. For 2° activation studies, cells can be activated for three days with OVA₃₂₃₋₃₃₉-loaded B cells, rested for one day, then activated with OVA₃₂₃₋₃₃₉-loaded BMDCs for 0, 10, 20, and 30 minutes. All cells can be stained with fluorescent antibodies against phospho-Zap70, phospho-Vav, and LRRC8A.

### LRRC8A is Required for Effective T_{FH}-GC B Cell Crosstalk.

Rationale. CD4⁺ T follicular helper (T_{FH}) cells are essential for long-lived humoral immunity.³⁵ The differentiation of T_{FH} cells requires repeated antigen stimulation.^{36,37} Cognate interactions between antigen-presenting dendritic cells and naïve T cells in the lymph node interfollicular cortex upregulates ICOS, upregulating expression of the transcriptional repressor Bcl-6 and the chemokine receptor CXCR5.³⁸ Differentiating T_{FH} cells migrate into the lymphoid follicle for 2° activation by B cells. T_{FH} cells provide CD40- and ICOS-driven costimulation for germinal center (GC) B cell formation, isotype switching, somatic hypermutation, and plasma cell development.^{39,40} Reduced numbers of T_{FH} cells in patients with mutations in CD40L, CD40, and ICOS demonstrates the essentiality of TCR costimulation for T_{FH} differentiation.^{41,42}

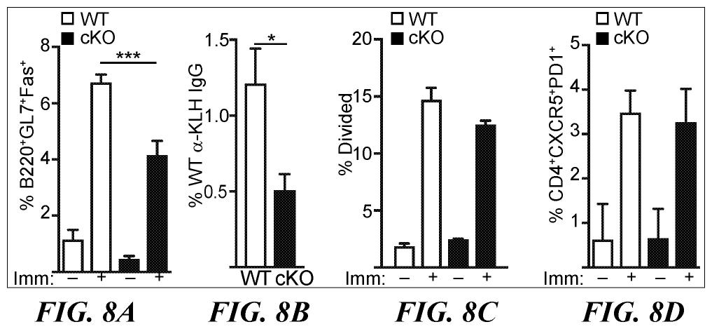

Having found that LRRC8A is important for the 2° activation of antigen-primed CD4⁻ T cells, we assessed the generation of GC B cells and T_{FH} cells in cKO mice immunized with the T-dependent antigen TNP-KLH. cKO mice had reduced B220⁺GL7⁺Fas⁺ GC B cells and anti-KLH IgG, despite normal T cell proliferation to MAI and percentages of CD4⁺CXCR5⁺PD1⁺ T_{FH} cells (FIG. 8), These findings demonstrate that T cell-specific deletion of LRRC8A impairs immoral immunity. This shows that LRRC8A enhances T_{FH} cell functions needed for the response to T dependent antigens.

Approach T_{FH} cell function can be assessed by quantifying T_{FH} cell-induced B cell class switching.⁴³ CD4⁺ICOS⁺CXCR5⁺CD25⁻CD19⁻ T_{FH} cells from WT or cKO mice can be co-cultured with WT CD19⁺ B cells in the presence of anti-CD3 and anti-IgM stimulation for six days, followed by flow cytometric analysis of cultured B cells for upregulation of the GC and activation marker GL7, the glucose transporter GLUT1, and sIgG1⁺. We can assess intracellular Bc16 and Ki67 expression in T_{FH} cells, both expressed in proliferating T_{FH} cells.

Despite similarities in signaling, CD28 and ICOS have distinct contributions to T_{FH} development and induce different patterns of gene expression important for T_{FH} cell development.⁴⁴ We can investigate the contribution of LRRC8A to the T_{FH} transcriptional profile. Seven days after TNP-KLH immunization of WT vs cKO mice, CD4⁺ICOS⁺CXCR5⁺CD25⁻CD19⁻ T_{FH} cells can be sorted from the draining lymph nodes for whole transcriptome analysis

Since GC formation is negatively regulated by CD4⁺FOXP3⁺PD1⁺CXCR5⁺ T follicular regulatory (Tfr) cells, we can determine whether LRRC8A affects the generation of Tfr cells.⁴⁵ We can quantify CD4⁺FOXP3⁺PD1⁺CXCR5⁺ Tfr cells in the draining lymph nodes of NP-OVA immunized WT vs cKO mice.

### Determine how LRRC8A Regulates T Cell Metabolism.

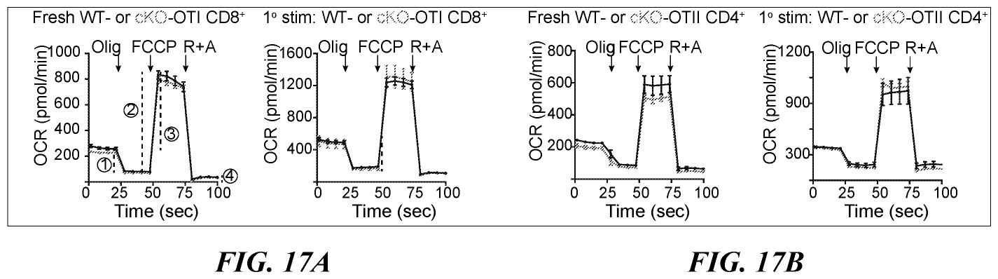

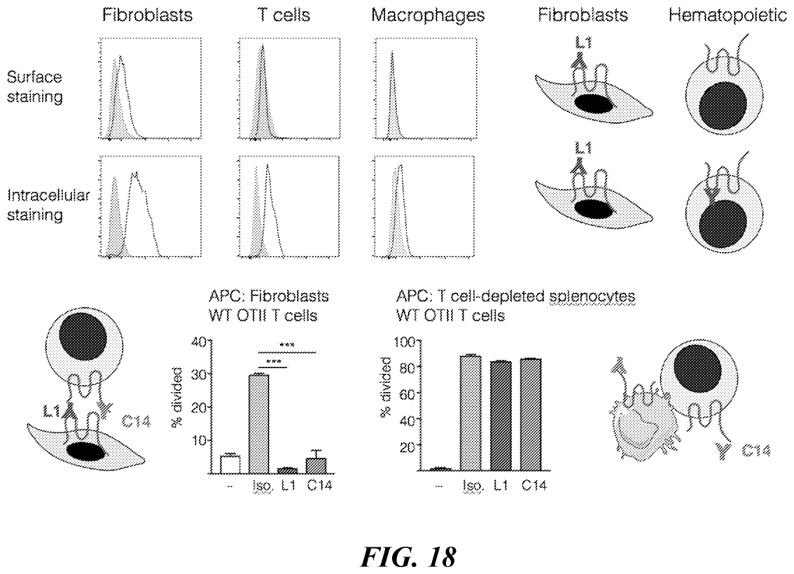

Preliminary data. Given the importance of mitochondria in T cell activation,⁴⁷ we measured the O₂ consumption rate (OCR) as an indicator of mitochondrial function. cKO CD4⁺ and CD8⁺ T cells had intact mitochondrial function before and after 1° activation (FIG. 17A, 17B).

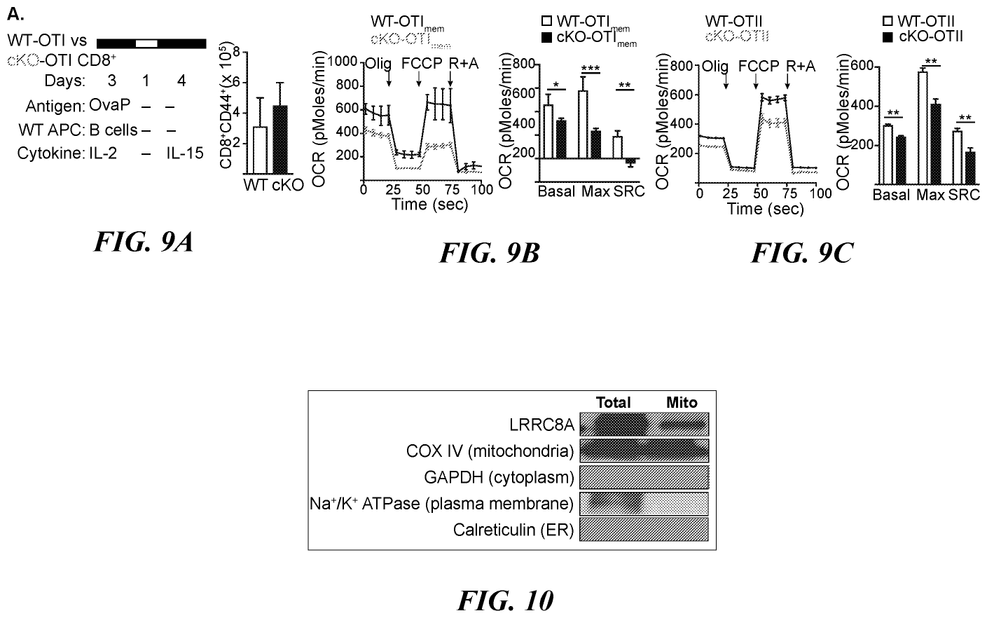

With sustained activation, T cells utilize glycolysis over oxidative metabolism to generate metabolic substrates for effector function.⁹ However, mitochondrial function and oxidative metabolism are particularly important for CD8⁺ memory and CD4⁺ T effector cells.^{48,49} We utilized an established in vitro approach to generate equivalent proportions of CD44⁺CD62L^{hi} central and CD44⁺CD62L^{lo} effector/memory CD8⁺ T cells, which were comparable between cKO and WT mice (FIG. 9A). However, cKO-OTI CD8⁺CD44^{hi} T cells had decreased basal and maximal OCR, as well as complete loss of the spare respiratory capacity (SRC) (FIG. 9B). Similarly, the basal OCR, maximal OCR, and SRC of antigen-primed cKO-OTII CD4⁺ T cell undergoing secondary activation were reduced (FIG. 9C), indicating mitochondrial dysfunction.

We found that LRRC8A exists in purified mitochondria from WT CD3⁺ T cells (FIG. 10). Since leucine-rich repeats facilitate interactions among proteins, we generated a fusion protein comprised of the leucine rich C-terminus of LRRC8A and GST (GST-LRRC8A) to identify potential interacting proteins using tandem mass spectrometry analysis. GST-LRRC8A pulled down the a subunit of the electron transfer flavoprotein and two subunits of the mitochondrial ATP synthase, all of which are part of the electron transport chain in the inner mitochondrial membrane (Table 2).^{50,51}

Table 2. The highest-ranking mitochondrial peptides binding to GST-LRRC8A, identified by LC-MS/MS. An unused score of >2 corresponds to >99% identity confidence based on the number of unique peptides in each protein.

TABLE 2

| | | Unused | % residues with |
|---|---|---|---|
| Protein | Function | Score | >95% conf. |
| Electron transfer flavoprotein, | Transfers electrons to Complex | 11.55 | 29.4 |
| α subunit | III of the electron transport | | |
| | chain | | |
| ATP synthase, O subunit | The oligomycin-sensitive | 8.44 | 31.9 |
| | subunit of ATP synthase | | |
| ATP synthase, β chain | The β subunit of the ATP | 5.94 | 12.5 |
| | synthase | | |

Summary We identified a novel function for LRRC8A as a mitochondrial regulator of oxidative metabolism in activated T cells. Work presented herein can determine how LRRC8A regulates the mitochondrial function and metabolic flexibility of antigen-primed CD4⁺and CD8⁺ T cells. T cell metabolism is an effective target for the treatment of autoimmunity, GVHD, and malignancies, all of which are relevant to patients with PIDs.¹²

LRRC8A Associates with the Electron Transport Chain and Enhances its Function.

Rationale. This work can delineate the mitochondrial sub-localization of LRRC8A, verify associated mitochondrial proteins, and assess the contribution of LRRC8A to electron transport chain function.

Approach. Mitochondria have four compartments (Table 3). While the porous outer membrane permits the free flow of ions and small molecules, the inner mitochondrial membrane is a tight diffusion barrier with ion selectivity that maintains its membrane potential. The inner membrane contains the electron transport chain, which fuels oxidative phosphorylation. To determine the sub-mitochondrial localization of LRRC8A, we can fractionate purified mitochondria from resting WT and cKO CD4⁺ and CD8⁺ T cells using a sucrose step gradient, differential centrifugation, and centrifugal concentration.⁵² Lysates of mitochondrial fractions can be immunoblotted with antibodies against LRRC8A and fraction-specific proteins (Table 3).

TABLE 3
Proteins specific for sub-mitochondrial fractions.

| Mitochondrial fraction | Fraction-specific protein |
|---|---|
| Outer mitochondrial membrane | Tom20 |
| Intermembrane space | Cytochrome c |
| Inner mitochondrial membrane | Tim17 |
| Mitochondrial matrix | Clp proteinase proteolytic subunit |

The electron transport chain is comprised of 5 complexes. We can immunoblot the 5 complexes in WT vs cKO CD4⁺ and CD8⁺ T cells at rest and after 1° and 2° activation. To identify the mitochondrial proteins associated with LRRC8A, we can perform large-scale purification mitochondria from WT Jurkat T cells, followed by immunoprecipitation of LRRC8A complexes and analysis with high resolution tandem mass spectrometry.

Electron transport chain function can be assessed using the Seahorse XF Real-Time ATP rate assay (Agilent), a kinetic assay that quantifies ATP production by simultaneously measuring mitochondrial respiration and the glycolytic rate. Intracellular ATP content can be assessed by quantifying luminescence after loading cells with luciferin and a thermostable luciferase (CellTiter-Glo Luminescent Cell Viability Reagent, Promega). These studies can be performed in WT-OTII vs cKO-OTII CD4⁺ at rest and after 2° activation, and in naïve vs in vitro differentiated memory WT-OTI vs cKO-OTI CD8⁺ T cells

LRRC8A preserves mitochondrial integrity during the cognate antigen response.

Rationale. During T cell activation, electron leakage from the electron transport chain generates reactive oxygen species (ROS).⁵⁸ ROS accumulation induces opening of mitochondrial permeability transition pores in the inner membrane, permitting water influx from the cytosol into the mitochondrial matrix.⁵⁹ Left unchecked, mitochondrial swelling due to hypotonic stress leads to dysfunction and eventual rupture.

In the plasma membrane, LRRC8A constitutes the essential pore-forming subunit of the volume-regulated anion channel (VRAC). Osmotic stress activates VRAC opening, leading to the efflux of anions and small solutes, water outflow, and relief from cellular swelling. Having shown that LRRC8A localizes to the mitochondria, we demonstrate that LRRC8A is also important for maintaining mitochondrial volume during the hypo-osmotic changes that occur during activation of antigen-primed cells.

Approach. Within 30 minutes of T cell activation, reactive oxygen species are detectable by flow cytometry, followed by changes in mitochondrial membrane potential, morphology, and mass.^{9,58} We can assess mitochondrial ROS, membrane potential, and mass after 2° activation of antigen-primed CD4⁺ T cells from WT-OTII vs cKO-OTII mice (Table 4). Cell viability can be assessed with Annexin V⁺ and fixable viability dye at each time point. The same studies can be done for antigen-primed CD8⁺CD44⁺ T_{eff/mem} cells.

Table 4. FACS analysis mitochondrial integrity. The 2 major mitochondrial ROS species, O⁻₂ and H₂O₂, are detected by MitoSOX Red and Mito Peroxy Yellow 1, respectively. CMTMRox accumulation correlates with membrane potential, while Mitotracker Green binds mitochondria independent of membrane potential.^{60,61}

TABLE 4

| Time after 2° stimulation Expected | | |
|---|---|---|
| with Ova peptide-loaded | | |
| WT B cells | outcome in WT cells | Reagent |
| 30 minutes | ↑↑ Mitochondrial ROS | MitoSOX Red and Mito Peroxy |
| | | Yellow 1 |
| 6 hours | ↑ Mitochondrial ROS | MitoSOX Red and Mito Peroxy |
| | ↑ Mito. membrane potential | Yellow 1 |
| | | CMTMRos |
| 24 hours | Normalized mitochondrial | MitoSOX Red and Mito Peroxy |
| | ROS | Yellow 1 |
| | Normal mito. membrane | CMTMRos |
| | potential | Mitotracker Green |
| | ↑ Total mitochondrial mass | |

Costimulatory molecules also influence metabolic function by regulating mitochondrial structure.⁶² CD28 costimulation of central memory T cells leads to densely packed mitochondrial cristae, enabling proximity among electron transport chain complexes for efficient oxidative phosphorylation.^{25,63} In contrast, the expanded shape of cristae in the mitochondria of effector T cells reduces electron transport chain efficiency, thus favoring aerobic glycolysis over oxidative phosphorylation.⁶⁴ To determine if LRRC8A affects mitochondrial remodeling, we can use transmission electron microscopy to assess the ultrastructure of mitochondria in antigen-primed CD4⁺ T cells from WT-OTII vs cKO-OTII mice at 24 and 48 hours after secondary activation. The same can be done for antigen-primed WT-OTI or cKO-OTI CD8⁺CD44⁺ T_{eff/mem} cells.

During the generation of antigen-experienced T cells, mitophagy removes damaged mitochondria.^{64,65} The voltage-dependent anion channel, which is distinct from the LRRC8A-containing VRAC channel, resides in the outer mitochondrial membrane and recruits proteins essential for mitophagy.^{66,67} Damage to the outer mitochondrial membrane allows proteins in the inner mitochondrial membrane to enhance mitophagy, partly by recruiting the autophagosomal protein LC3.⁶⁸ To determine if LRRC8A is important for mitophagy, WT-OTII vs cKO-OTII CD4⁺ T cells can undergo 2° activation (FIG. 3A), followed by staining for LC3-II and Mitotracker green after 18, 24, 48, and 72 hours of stimulation. Laser scanning confocal microscopy can be used to quantify mitophagy, as shown by the colocalization of LC3-II with mitochondria.⁶⁹ As a complementary approach, we can immunoblot cell lysates for prohibitin 1 and prohibitin 2, which degrade during mitophagy.⁶⁸

### LRRC8A is Critical for Metabolic Flexibility of T Cells

Rationale. After secondary stimulation, antigen-experienced cKO-OTII CD4⁺ T cells had reduced expression of multiple PI3K-regulated genes important for glycolysis (Table 5).

Table 5. Expression of PI3K-regulated glycolysis genes in cKO-OTII CD4⁺ T cells after 2° activation with OVA₃₂₃₋₃₃₉ presenting, IFN-γ treated fibroblasts.

TABLE 5

| | | Fold change relative | |
|---|---|---|---|
| Gene | Function in glycolysis | to WT | FDRp value |
| Slc16a3 | Monocarboxylic acid transporter 4 exports | −15.7 | 0.014 |
| | glycolysis byproducts | | |
| Irf4 | Transcription factor that upregulates glycolytic | −6.77 | 0.041 |
| | pathways | | |
| Slc2a3 | Glucose transporter (Glut3) | −4.00 | 0.049 |
| Pkm2 | Pyruvate kinase catalyzes the transfer of | −1.83 | 0.025 |
| | phosphoryl groups from phosphoenolpyruvate to | | |
| | ADP, thereby generating ATP | | |
| Tpi1 | Triosephosphate isomerase generates | −1.96 | 0.024 |
| | glyceraldehyde-3-phosphate, an intermediate | | |
| | metabolite of glycolysis | | |
| Slc2a1 | Glucose transporter (Glut1) | −1.74 | 0.032 |

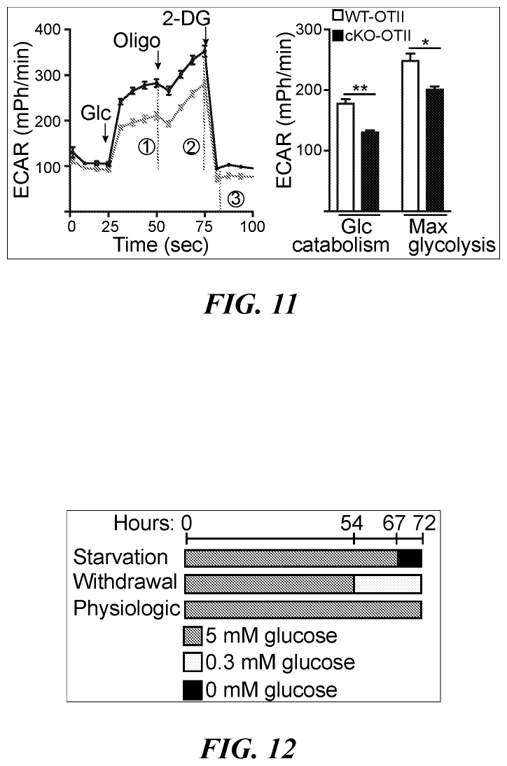

To validate these findings, we performed a preliminary experiment that showed that antigen-primed cKO-OTII CD4⁺ T cells had reduced glucose catabolism as well as maximal glycolytic capacity (FIG. 11).

The capacity for utilizing multiple metabolic pathways confers metabolic flexibility for T cell activation during nutrient restriction or hypoxia induced by infection or chronic inflammation. Having shown roles for LRRC8A in oxidative metabolism and glycolysis, 2.3 demonstrated that LRRC8A provides a metabolic advantage to effector T cells in glucose-poor or hypoxic microenvironments.

Approach. To complement the inventors' studies of glycolysis in CD4⁺ T cells (FIG. 11), we can measure the extracellular acidification rate of WT-OTI or cKO-OTI CD8⁺ T cells at rest and after 1° and 2° activation. Additionally, we can perform metabolic profiling of WT-OTII vs cKO-OTII CD4⁺ as well as WT-OTI vs cKO-OTI CD8⁺ T cells at rest and after 1° and 2° activation. Activated cells can be snap frozen after washing with ammonium carbonate. Metabolites can be extracted with hot 70% ethanol, followed by selective reaction monitoring and mass spectrometry.

Inflammation and infection generate metabolically restrictive conditions in tissues. The metabolic flexibility of intact T cells prioritizes pathways capable of generating substrates for survival and function even under nutrient-limited conditions.^{10,71} T cell differentiation and effector function is also influenced by nutrient availability, as demonstrated by the increased proliferation, survival, and function of effector CD8⁺ T cells under hypoxic conditions.⁷²⁻⁷⁴ The dual contribution of LRRC8A to glycolysis and oxidative phosphorylation demonstrates a potential role for LRRC8A in the maintenance of metabolic flexibility. Established approaches for assessing the metabolic flexibility of T cells utilize culture conditions with low glucose concentrations (<5.5 mM) or hypoxia (1% O₂) to limit glycolysis or mitochondrial aerobic metabolism, respectively. These conditions mimic those found in inflamed tissues.^{25,71,72,75} WT-OTII vs cKO-OTII CD4⁺ T cells can undergo 1° activation with: (1) acute glucose starvation for the last 5 hrs, (2) glucose withdrawal for the last 18 hours, or (3) physiologic glucose concentrations (FIG. 12). A subset of cells can then undergo 2° activation under starvation, withdrawal, or physiologic glucose concentrations. Activated cells can be assessed by FACS for viability, proliferation, expression of the activation markers CD25 and CD69, and IL-2 and IFN-y secretion by ELISA. WT-OTI vs cKO-OTI CD8⁺ T cells can be similarly activated and assessed. For hypoxic cultures, APCs and T cells can be preincubated separately in 1% 02 for 24 hours, followed by co-culture for 1° or 2° activation in 1% O₂.⁷²

Targeting LRRC8A in Diseases with Self-Reactive T Cells.

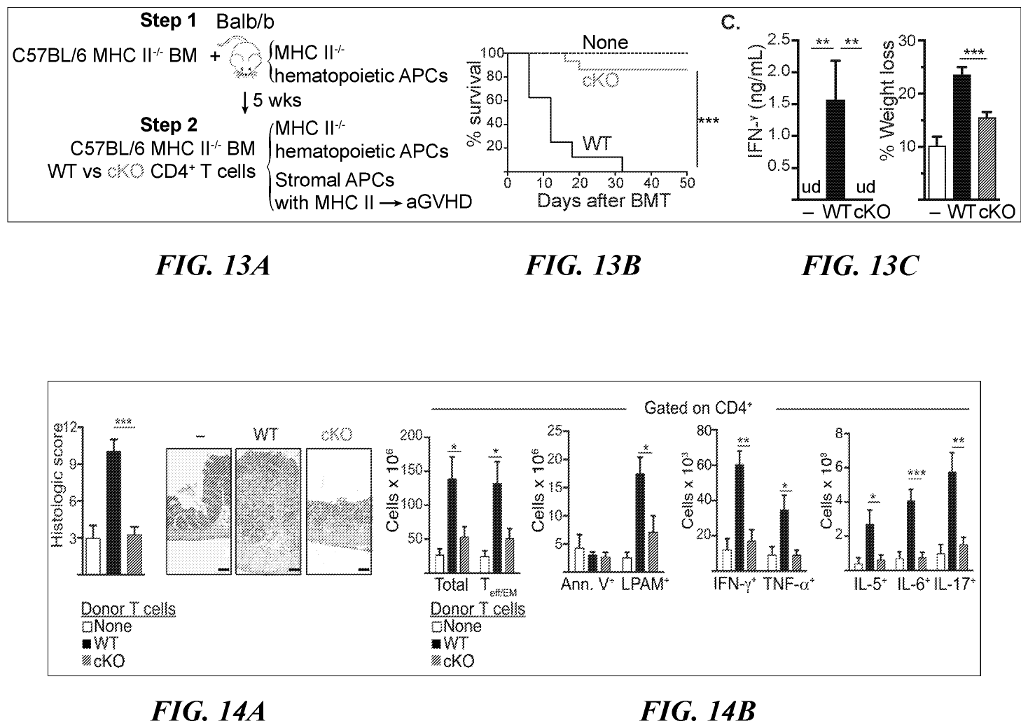

Preliminary data. To determine the biologic significance of LRRRC8A-driven T cell activation, we utilized an established model of acute GVHD induced by donor C57BL/6 CD4⁺ T cells into Balb/b recipients. These strains share major histocompatibility antigens, but have mismatched minor histocompatibility antigens. Inflamed recipient stromal cells are critical for presenting minor histocompatibility antigens to donor CD4⁺ T cells.^{78,79} In contrast, the deletion of recipient dendritic cells, B cells, Langerhans cells, or macrophages fails to reduce and can instead worsen acute GVHD.⁸⁰⁻⁸³ In this model, MHC class II is expressed solely on non-hematopoietic cells. This is achieved by reconstituting irradiated Balb/B (H-2^{b}) recipients with T cell-depleted bone marrow from MHC class II-deficient mice (B6.H2-Ab1^{-/-}).^{78,79} Five weeks after reconstitution, aGVHD was induced in irradiated chimeras by transplanting WT or cKO C57BL/6 CD4⁺ T cells with T cell-depleted MHC class II-deficient bone marrow (FIG. 13A). No recipients of donor WT CD4⁺ T cells survived beyond 32 days post-transplantation, while 78% of mice who received donor cKO CD4⁺ T cells remained alive at 50 days post-transplantation (FIG. 13B). Recipients of conditional LRRC8A-deficient donor CD4⁺ T cells had undetectable serum IFN-γ at 7 days post-transplantation and less weight loss, consistent with reduced GVHD (FIG. 13C).

Recipients of donor WT CD4⁺ T cells had lymphocytic infiltrate in the lamina propria (LP) and evidence of crypt destruction not seen in recipients of donor cKO CD4⁺ T cells (FIG. 14A). Flow cytometric analysis of T cells isolated from the colonic LP showed that recipients of cKO donor CD4⁺ T cells had fewer infiltrating CD4⁺ T cells, with fewer CD4⁺CD62^{lo}CD44^{hi} effector/effector memory T cells, decreased expression of the alloreactive T cell gut homing integrin LPAM (α4β7), and reduced expression of intracellular cytokines important for the pathogenesis of acute GVHD (FIG. 14B).

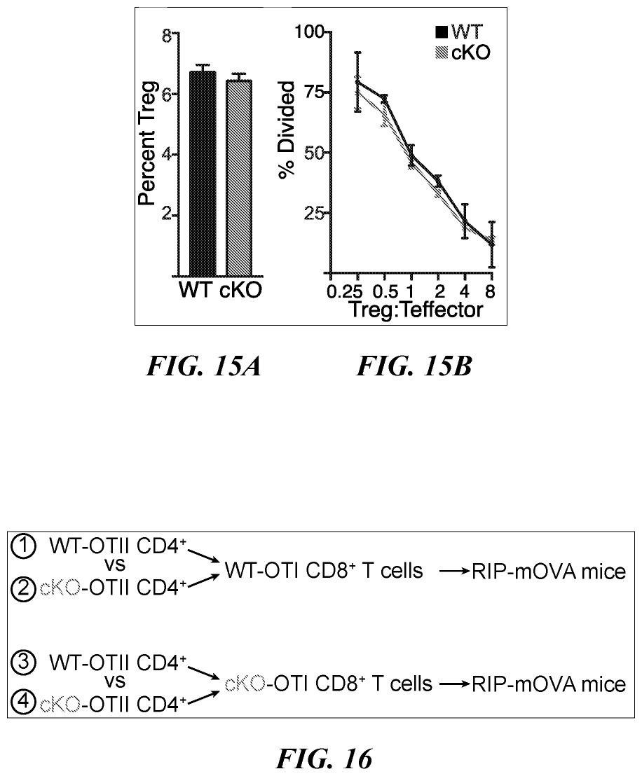

Notably, cKO and WT donors had comparable numbers and in vitro suppressive function of regulatory T cells, which are important negative regulators of acute GVHD (FIG. 15).

Summary LRRC8A on donor CD4⁺ T cells is essential for acute GVHD resulting from mismatched minor histocompatibility antigens presented by non-hematopoietic APCs. Ongoing studies funded by Dr. Chou's K08 grant are studying the role of LRRC8A to GVHD due to mismatched major histocompatibility antigens. Aim 3 builds on this work by assessing the contribution of LRRC8A to CD4⁺ and CD8⁺ T cell-driven diseases relevant to patients with PIDs.

LRRC8A-Deficient T Cells have Reduced Capacity for Inducing Autoimmunity.

Rationale. Autoreactive effector and memory T cells mediate autoimmunity in a diversity of PIDs, including IPEX syndrome, deficiency of the E3 ubiquitin ligase ITCH, CTLA4 haploinsufficiency, and LRBA deficiency.^{84,85} Despite differences in monogenic defects, impaired regulation of effector T cells is a common final pathway leading to organ destruction. While glycolysis supplies metabolites needed for effector T cell functions, oxidative metabolism fuels the survival and function of memory T cells.^{9,86} Antigen-primed CD4⁺ T cells enhance the survival and proliferation of memory CD8⁺ T cells, while lowering the threshold number of CD8⁺ T cells needed for end-organ damage in models of autoimmunity.⁸⁷⁻⁸⁹ Having found impaired metabolism and activation in antigen-primed cKO CD4⁺ and cKO CD8⁺ memory T cells, we can now determine if LRRC8A deficiency reduces the progression of T cell-driven Type I diabetes.

Approach. We can first utilize a model of Type I diabetes induced by adoptively transferred pre-activated effector/memory CD8⁺ T cells. The pancreatic islet βcells of the C57BL/6 rat insulin promoter (RIP)-OVA^{lo} mice (Jackson Laboratory) express low levels of OVA (0.03 ng OVA/μg islet cell protein) that fail to activate adoptively transferred naïve CD8⁺ cells.⁹⁰ However, the adoptive transfer of in vitro-generated effector/memory CD8⁺ T cells destroys islet cells, reflecting the low antigen threshold required for the re-activation of antigen-primed, effector/memory CD8⁺ T cells.⁹¹ A high (4×10⁷) or low (2×10⁷) dose of adoptively transferred effector/memory CD8⁺ T cells induces diabetes within 10 and 23 days, respectively.⁹¹ We can generate antigen-primed effector/memory CD8⁺ T cells by co-culturing WT-OTI vs cKO-OTI CD8⁺ T cells with OVa₂₅₇₋₂₆₄ and IL-2 for 3 days, followed by washing and culture in IL-15 for 2 additional days.⁹¹ We can purify CD8⁺CD44⁺CD62L⁺ central memory and CD8⁺CD44⁺CD62L⁻ effector/effector memory T cells, followed by adoptive transfer at a 1:1 ratio into RIP-OVA^{lo} recipients at high vs low doses. Blood glucose will be measured every 2 days. We can assess islet destruction by histological examination of pancreatic sections using an established scoring system.⁹² T cells in the pancreas and draining lymph nodes can be isolated, followed by FACS analysis for cell viability (Annexin V⁺ and fixable viability dye), proliferation (Ki-67), and intracellular IL-2, IFN-γ, granzyme B, and perforin. The oxidative metabolism and glycolytic rate of T cells from draining pancreatic lymph nodes can be assessed by extracellular flux analysis.

Since autoimmunity in patients arises from the crosstalk between CD4⁺ and CD8⁺ T cells, we can also utilize a model of Type I diabetes in which CD4⁺ help modulates the damage induced by self-reactive CD8⁺ T cells. C57BL/6 RIP-mOVA mice (Jackson Laboratory) express high levels of OVA (2.2 ng OVA/μg islet cell protein) on pancreatic β cells and renal proximal tubular cells.⁹⁰ Although high numbers of adoptively transferred OT-I CD8⁺ T cells are sufficient for inducing diabetes in RIP-mOVA mice, the concomitant addition of OT-II CD4⁺ T cells lowers the number of OT-I CD8⁺ T cells required to induce diabetes.⁸⁹ We can adoptively transfer combinations of WT vs cKO OT transgenic T cells (FIG. 16). Blood glucose, histology, and analysis of infiltrating T cells can be assessed as described above.

### LRRC8A Promotes Host Immunity Against Viral Infection.

Rationale. To complement the inventors' in vitro studies demonstrating impaired cytotoxicity in cKO CD8⁺ T cells (FIG. 5D), we can investigate the contribution of LRRC8A to immunity against LCMV. Infections with Armstrong LCMV elicit an acute CD8⁺ T cell response, leading to viral clearance in ˜1 week and long-lasting immunity. In contrast, LCMV clone 13 has two mutations that increase its binding to host cells and its rate of replication, leading to viral persistence and CD8⁺ T cell exhaustion.⁹⁵ Using these model pathogens, the inventors investigated the susceptibility to viral infections that may accompany therapeutic strategies targeting LRRC8A in T cells.

Approach. WT or cKO mice can be i.p. injected with 2×10⁵ plaque-forming units of LCMV Armstrong (available from European Virus Archive Goes Global) or 4×10⁶ pfu of LCMV clone 13 (gift of Dr. Raif Geha, Boston Children's Hospital) into WT or cKO mice. The survival and weight of infected mice can be followed for 14 (Armstrong) or 50 days (clone 13). The inventors' studies of the immune response in the acute and chronic phases of LCMV infection are detailed in Table 6.⁹⁶⁻¹⁰⁰

TABLE 6

| Assessment of T cell | Days after | Days after |
|---|---|---|
| responses after LCMV infection, | Armstrong injection | clone 13 injection |
| Viral titers in serum, spleen, and | 2, 4, 7, 10 | 4, 7, 20, 50 |
| liver by plaque assay | | |
| Numbers of gp33-tetramer⁺CD8⁺ and | 5, 10, 15, 20 | 5, 10, 20, 50 |
| gp66-tetramer⁺CD4⁺ | | |
| Proliferation of gp33-tetramer⁺CD8⁺ | 5, 10, 15, 20 | 5, 10, 20, 50 |
| and gp66-tetramer⁺CD4⁺ T cells to | | |
| gp33 or gp66, respectively | | |
| IFN-γ, TNF-α, IL-2 in gp33- | 5, 10, 15, 20 | 5, 10, 20, 50 |
| tetramer⁺CD8⁺ and gp66- | | |
| tetramer⁺CD4⁺T cells to gp33 or | | |
| gp66, respectively | | |
| Exhaustion markers (PD-1, CD160, | 7, 20 | 10, 20, 50 |
| CD272) on gp33-tetramer⁺CD8⁺ and | | |
| gp66-tetramer⁺CD4⁺ cells | 7, 20 | 10, 20, 50 |
| Total IgG and LCMV-specific IgG | 7, 20 | 10, 20, 50 |
| B220⁺Fas⁺GL7⁺ GC B cells and | 7, 20 | 10, 20, 50 |
| GLT⁻CD38⁺IgD⁻IgM⁻ memory B | | |
| cells | | |

It is specifically contemplated herein that that this can be followed by increased viral loads, impaired viral clearance, and reduced survival during the chronic phase of infection.

### CONCLUSION

Organ damage from activated T cells is a life-threatening consequence of multiple PIDs. In defining how LRRC8A regulates T cell activation and metabolism within the contexts of autoimmunity and infection, the inventors developed a new mechanistic framework for modulating organ damage in patients with PIDs.

### REFERENCES

1. Grimbacher, B., Warnatz, K., Yong, P. F. K., Korganow, A.-S. & Peter, H.-H. The crossroads of autoimmunity and immunodeficiency: Lessons from polygenic traits and monogenic defects. J. Allergy Clin. Immunol. 137, 3-17 (2016).
2. Laberko, A. & Gennery, A. R. Clinical considerations in the hematopoietic stem cell transplant management of primary immunodeficiencies. Expert Rev. Clin. Immunol. 14, 297-306 (2018).
3. Kumar, L. et al. Leucine-rich repeat containing 8A (LRRC8A) is essential for T lymphocyte development and function. J. Exp. Med. 211, 929-942 (2014).
4. Qiu, Z. et al. SWELL1, a Plasma Membrane Protein, Is an Essential Component of Volume-Regulated Anion Channel. Cell 157, 447-458 (2014).
5. Voss, F. K., Ullrich, F., Munch, J., Lazarow, K. & Lutter, D. Identification of LRRC8 Heteromers as an Essential Component of the Volume-Regulated Anion Channel VRAC. Sci. N. Y. NY (2014).
6. Platt, C. D. et al. Leucine-rich repeat containing 8A (LRRC8A)-dependent volume-regulated anion channel activity is dispensable for T-cell development and function. J. Allergy Clin. Immunol. 140, 1651-1659.e1 (2017).
7. Schmidt, R. E., Grimbacher, B. & Witte, T. Autoimmunity and primary immunodeficiency: two sides of the same coin? Nat. Rev. Rheumatol. 14, 7-18 (2018).
8. Mechanism-Based Strategies for the Management of Autoimmunity and Immune Dysregulation in Primary Immunodeficiencies—ClinicalKey. Available at: https://www-clinicalkey-com.ezp-prod1.hul.harvard.edu/#!/content/playContent/1-s2.0-S2213219816303798?returnurl=https:%2F%2Flinkinghub.elsevier.com %2Fretrieve %2Fpii %2FS2213219816303798%3Fshowall%3Dtrue&referrer=https:%2F%2Fwww.ncbi.nlm.nih.gov%2F. (Accessed: 2 Oct. 2018)
9. Bantug, G. R., Galluzzi, L., Kroemer, G. & Hess, C. The spectrum of T cell metabolism in health and disease. Nat. Rev. Immunol. 18, 19-34 (2018).
10. Buck, M. D., Sowell, R. T., Kaech, S. M. & Pearce, E. L. Metabolic Instruction of Immunity. Cell 169, 570-586 (2017).
11. Buck, M. D., O'Sullivan, D. & Pearce, E. L. T cell metabolism drives immunity. J. Exp. Med. 212, 1345-1360 (2015).
12. O'Sullivan, D. & Pearce, E. L. Targeting T cell metabolism for therapy. Trends Immunol. 36, 71-80 (2015).
13. Kumar, R. et al. Increased Sensitivity of Antigen-Experienced T Cells through the Enrichment of Oligomeric T Cell Receptor Complexes. Immunity 35, 375-387 (2011).
14. Umetsu, D. T. et al. Human dermal fibroblasts present tetanus toxoid antigen to antigen-specific T cell clones. J. Clin. Invest. 76, 254-260 (1985).
15. Umetsu, D. T., Katzen, D., Jabara, H. H. & Geha, R. S. Antigen presentation by human dermal fibroblasts: activation of resting T lymphocytes. J. Immunol. Baltim. Md 1950 136, 440-445 (1986).
16. Bossaller, L. et al. ICOS Deficiency Is Associated with a Severe Reduction of CXCR5+CD4 Germinal Center Th Cells. J. . . . (2006).
17. Ma, C. S. et al. Unique and shared signaling pathways cooperate to regulate the differentiation of human CD4+ T cells into distinct effector subsets. J. Exp. Med. 213, 1589-1608 (2016).
18. Byun, M. et al. Inherited human OX40 deficiency underlying classic Kaposi sarcoma of childhood. J. Exp. Med. 210, 1743-1759 (2013).
19. Flaherty, S. & Reynolds, J. M. Mouse Naïve CD4+ T Cell Isolation and In vitro Differentiation into T Cell Subsets. J. Vis. Exp. JoVE (2015). doi:10.3791/52739
20. Wirth, T. C. et al. Repetitive Antigen Stimulation Induces Stepwise Transcriptome Diversification but Preserves a Core Signature of Memory CD8+ T Cell Differentiation. Immunity 33, 128-140 (2010).
21. Baas, M. et al. TGFβ-dependent expression of PD-1 and PD-L1 controls CD8+ T cell anergy in transplant tolerance. eLife 5, e08133 (2016).
22. Yee, C. S. et al. Recurrent viral infections associated with a homozygous CORO1A mutation that disrupts oligomerization and cytoskeletal association. J. Allergy Clin. Immunol. 137, 879-88.e2 (2015).
23. Chou, J. et al. A novel mutation in ORAI1 presenting with combined immunodeficiency and residual T-cell function. J. Allergy Clin. Immunol. 136, 479-82.e1 (2015).
24. Badran, Y. R. et al. Combined immunodeficiency due to a homozygous mutation in ORAI1 that deletes the C-terminus that interacts with STIM 1. Clin. Immunol. 166-167, 100-102 (2016).
25. Klein Geltink, R. I. et al. Mitochondrial Priming by CD28. Cell (2017). doi:10.1016/j.cell.2017.08.018
26. Kawalekar, O. U. et al. Distinct Signaling of Coreceptors Regulates Specific Metabolism Pathways and Impacts Memory Development in CAR T Cells. Immunity 44, 380-390 (2016).
27. Bellucci, A., Fiorentini, C., Zaltieri, M., Missale, C. & Spano, P. The “In Situ” Proximity Ligation Assay to Probe Protein—Protein Interactions in Intact Tissues. in Exocytosis and Endocytosis (ed. Ivanov, A. I.) 397-405 (Springer New York, 2014). doi:10.1007/978-1-4939-0944-5_27
28. Sawada, A. et al. A congenital mutation of the novel gene LRRC8 causes agammaglobulinemia in humans. J. Clin. Invest. 112, 1707-1713 (2003).
29. Woodall, N. B., Yin, Y. & Bowie, J. U. Dual-topology insertion of a dual-topology membrane protein. Nat. Commun. 6, (2015).
30. Bowens, N. H., Dohare, P., Kuo, Y.-H. & Mongin, A. A. DCPIB, the Proposed Selective Blocker of Volume-Regulated Anion Channels, Inhibits Several Glutamate Transport Pathways in Glial Cells. Mol. Pharmacol. 83, 22-32 (2013).
31. Friard, J. et al. Comparative Effects of Chloride Channel Inhibitors on LRRC8/VRAC-Mediated Chloride Conductance. Front. Pharmacol. 8, (2017).
32. Feske, S., Wulff, H. & Skolnik, E. Y. Ion Channels in Innate and Adaptive Immunity. Annu. Rev. Immunol. 33, 291-353 (2015).
33. Cahalan, M. D. & Chandy, K. G. The functional network of ion channels in T lymphocytes. Immunol. Rev. 231, 59-87 (2009).
34. Vivar, O. I. et al. IFT20 controls LAT recruitment to the immune synapse and T-cell activation in vivo. Proc. Natl. Acad. Sci. 113, 386-391 (2016).
35. Crotty, S. T Follicular Helper Cell Differentiation, Function, and Roles in Disease. Immunity 41, 529-542 (2014).
36. Deenick, E. K. et al. Follicular helper T cell differentiation requires continuous antigen presentation that is independent of unique B cell signaling. Immunity 33, 241-253 (2010).
37. Maceiras, A. R. et al. T follicular helper and T follicular regulatory cells have different TCR specificity. Nat. Commun. 8, (2017).
38. Trüb, M. et al. Heterogeneity of Phenotype and Function Reflects the Multistage Development of T Follicular Helper Cells. Front. Immunol. 8, (2017).
39. Schmitt, N. et al. Human Dendritic Cells Induce the Differentiation of Interleukin-21-Producing T Follicular Helper-like Cells through Interleukin-12. Immunity 31, 158-169 (2009).
40. Lüthje, K. et al. The development and fate of follicular helper T cells defined by an IL-21 reporter mouse. Nat. Immunol. 13, 491-498 (2012).
41. Ma, C. S. et al. Monogenic mutations differentially affect the quantity and quality of T follicular helper cells in patients with human primary immunodeficiencies. J. Allergy Clin. Immunol. (2015). doi:10.1016/j.jaci.2015.05.036
42. Cicalese, M. P. et al. Circulating Follicular Helper and Follicular Regulatory T Cells Are Severely Compromised in Human CD40 Deficiency: A Case Report. Front. Immunol. 9, (2018).
43. Sage, P. T. & Sharpe, A. H. In Vitro Assay to Sensitively Measure Tfr Suppressive Capacity and Tfh Stimulation of B Cell Responses. in T follicular Helper Cells: Methods and Protocols (eds. Espéli, M. & Linterman, M.) 151-160 (Springer New York, 2015). doi:10.1007/978-1-4939-2498-1_13
44. Weber, J. P. et al. ICOS maintains the T follicular helper cell phenotype by down-regulating Krüppel-like factor 2. J. Exp. Med. 212, 217-233 (2015).
45. Sage, P. T. et al. Suppression by T_{FR} cells leads to durable and selective inhibition of B cell effector function. Nat. Immunol. 17, 1436-1446 (2016).
46. Reinhardt, R. L., Liang, H.-E. & Locksley, R. M. Cytokine-secreting follicular T cells shape the antibody repertoire. Nat. Immunol. 10, 385-393 (2009).
47. Almeida, L., Lochner, M., Berod, L. & Sparwasser, T. Metabolic pathways in T cell activation and lineage differentiation. Semin. Immunol. 28, 514-524 (2016).
48. Cao, Y., Rathmell, J. C. & Macintyre, A. N. Metabolic Reprogramming towards Aerobic Glycolysis Correlates with Greater Proliferative Ability and Resistance to Metabolic Inhibition in CD8 versus CD4 T Cells. PLoS ONE 9, e104104 (2014).
49. Kolev, M. et al. Complement Regulates Nutrient Influx and Metabolic Reprogramming during Th1 Cell Responses. Immunity 42, 1033-1047 (2015).
50. Jonckheere, A. I., Smeitink, J. A. M. & Rodenburg, R. J. T. Mitochondrial ATP synthase: architecture, function and pathology. J. Inherit. Metab. Dis. 35, 211-225 (2012).
51. Zhang, J., Frerman, F. E. & Kim, J.-J. P. Structure of electron transfer flavoprotein-ubiquinone oxidoreductase and electron transfer to the mitochondrial ubiquinone pool. Proc. Natl. Acad. Sci. 103, 16212-16217 (2006).
52. Pallotti, F. & Lenaz, G. Isolation and Subfractionation of Mitochondria from Animal Cells and Tissue Culture Lines. in Methods in Cell Biology 80, 3-44 (Academic Press, 2007).
53. Rossi, M. N. et al. Mitochondrial Localization of PARP-1 Requires Interaction with Mitofilin and Is Involved in the Maintenance of Mitochondrial DNA Integrity. J. Biol. Chem. 284, 31616-31624 (2009).
54. Stan, T. et al. Mitochondrial Protein Import: Recognition of Internal Import Signals of BCS1 by the TOM Complex. Mol. Cell. Biol. 23, 2239-2250 (2003).
55. Holding, A. N. XL-MS: Protein cross-linking coupled with mass spectrometry. Methods 89, 54-63 (2015).
56. Chavez, J. D. et al. Chemical Crosslinking Mass Spectrometry Analysis of Protein Conformations and Supercomplexes in Heart Tissue. Cell Syst. 6, 136-141.e5 (2018).
57. Schweppe, D. K. et al. Mitochondrial protein interactome elucidated by chemical cross-linking mass spectrometry. Proc. Natl. Acad. Sci. 114, 1732-1737 (2017).
58. Sena, L. A. et al. Mitochondria Are Required for Antigen-Specific T Cell Activation through Reactive Oxygen Species Signaling. Immunity 38, 225-236 (2013).
59. Mootha, V. K. A reversible component of mitochondrial respiratory dysfunction in apoptosis can be rescued by exogenous cytochrome c. EMBO J. 20, 661-671 (2001).
60. Nagy, G., Koncz, A. & Perl, A. T Cell Activation-Induced Mitochondrial Hyperpolarization Is Mediated by Ca2+- and Redox-Dependent Production of Nitric Oxide. J. Immunol. Baltim. Md 1950 171, 5188-5197 (2003).
61. Zhang, B. et al. MicroRNA-23a Curbs Necrosis during Early T Cell Activation by Enforcing Intracellular Reactive Oxygen Species Equilibrium. Immunity 44, 568-581 (2016).
62. Cogliati, S., Enriquez, J. A. & Scorrano, L. Mitochondrial Cristae: Where Beauty Meets Functionality. Trends Biochem. Sci. 41, 261-273 (2016).
63. Cogliati, S. et al. Mitochondrial Cristae Shape Determines Respiratory Chain Supercomplexes Assembly and Respiratory Efficiency. Cell 155, 160-171 (2013).
64. Buck, M. D. et al. Mitochondrial Dynamics Controls T Cell Fate through Metabolic Programming. Cell 166, 63-76 (2016).
65. Botbol, Y., Guerrero-Ros, I. & Macian, F. Key roles of autophagy in the regulation of T-cell function. Eur. J. Immunol. 46, 1326-1334 (2016).
66. Ponnalagu, D. & Singh, H. Anion Channels of Mitochondria. Handb. Exp. Pharmacol. 240, 71-101 (2017).
67. Sun, Y., Vashisht, A. A., Tchieu, J Wohlschlegel, J. A. & Dreier, L. Voltage-dependent Anion Channels (VDACs) Recruit Parkin to Defective Mitochondria to Promote Mitochondrial Autophagy. J. Biol. Chem. 287, 40652-40660 (2012).
68. Wei, Y., Chiang, W.-C., Sumpter, R., Mishra, P. & Levine, B. Prohibitin 2 Is an Inner Mitochondrial Membrane Mitophagy Receptor. Cell 168, 224-238.e10 (2017).
69. Mizushima, N., Yoshimorim, T. & Levine, B. Methods in Mammalian Autophagy Research. Cell 140, 313-326 (2010).
70. Flanagan, L. A. et al. Filamin A, the Arp2/3 complex, and the morphology and function of cortical actin filaments in human melanoma cells. J. Cell Biol. 155, 511-518 (2001).
71. Dumitru, C., Kabat, A. M. & Maloy, K. J. Metabolic Adaptations of CD4+ T Cells in Inflammatory Disease. Front. Immunol. 9, (2018).
72. Xu, Y. et al. Glycolysis determines dichotomous regulation of T cell subsets in hypoxia. J. Clin. Invest. 126, 2678-2688 (2016).
73. Doedens, A. L. et al. Hypoxia-inducible factors enhance the effector responses of CD8⁺ T cells to persistent antigen. Nat. Immunol. 14, 1173-1182 (2013).
74. Finlay, D. K. et al. PDK1 regulation of mTOR and hypoxia-inducible factor 1 integrate metabolism and migration of CD8+ T cells. J. Exp. Med. 209, 2441-2453 (2012).
75. Blagih, J. et al. The Energy Sensor AMPK Regulates T Cell Metabolic Adaptation and Effector Responses In Vivo. Immunity 42, 41-54 (2015).
76. Nyamundanda, G., Gormley, I. C., Fan, Y., Gallagher, W. M. & Brennan, L. MetSizeR: selecting the optimal sample size for metabolomic studies using an analysis based approach. BMC Bioinformatics 14, 338 (2013).
77. Dohi, T., Beltrami, E., Wall, N. R., Plescia, J. & Altieri, D. C. Mitochondrial survivin inhibits apoptosis and promotes tumorigenesis. J. Clin. Invest. 114, 1117-1127 (2004).
78. Jones, S. C., Murphy, G. F., Friedman, T. M. & Korngold, R. Importance of minor histocompatibility antigen expression by nonhematopoietic tissues in a CD4+ T cell-mediated graft-versus-host disease model. J. Clin. Invest. 112, 1880-1886 (2003).
79. Koyama, M. et al. Recipient nonhematopoietic antigen-presenting cells are sufficient to induce lethal acute graft-versus-host disease. Nat. Med. 18, 135-142 (2012).
80. Li, H. et al. Profound Depletion of Host Conventional Dendritic Cells, Plasmacytoid Dendritic Cells, and B Cells Does Not Prevent Graft-versus-Host Disease Induction. J. Immunol. 188, 3804-3811 (2012).
81. Li, H. et al. Langerhans cells are not required for graft-versus-host disease. Blood 117, 697-707 (2011).
82. Matte-Martone, C. et al. Recipient B Cells Are Not Required For GVHD Induction. Biol. Blood Marrow Transplant. J. Am. Soc. Blood Marrow Transplant. 16, 1222-1230 (2010).
83. Rowe, V. et al. Host B cells produce IL-10 following TBI and attenuate acute GVHD after allogeneic bone marrow transplantation. Blood 108, 2485-2492 (2006).
84. Lo, B. et al. CHAI and LATAIE: new genetic diseases of CTLA-4 checkpoint insufficiency. Blood 128, 1037-1042 (2016).
85. Azizi, G. et al. Approach to the Management of Autoimmunity in Primary Immunodeficiency. Scand. J. Immunol. 85, 13-29 (2017).
86. van der Windt, G. J. W. et al. Mitochondrial Respiratory Capacity Is a Critical Regulator of CD8+ T Cell Memory Development. Immunity 36, 68-78 (2012).
87. Novy, P., Quigley, M., Huang, X. & Yang, Y. CD4 T Cells Are Required for CD8 T Cell Survival during Both Primary and Memory Recall Responses. J. Immunol. 179, 8243-8251 (2007).
88. Saout, C. L., Mennechet, S., Taylor, N. & Hernandez, J. Memory-like CD8+ and CD4+ T cells cooperate to break peripheral tolerance under lymphopenic conditions. Proc. Natl. Acad. Sci. 105, 19414-19419 (2008).
89. Friesen, T. J., Ji, Q. & Fink, P. J. Recent thymic emigrants are tolerized in the absence of inflammation. J. Exp. Med. jem.20151990 (2016). doi:10.1084/jem.20151990
90. Kurts, C. et al. CD8 T cell ignorance or tolerance to islet antigens depends on antigen dose. Proc. Natl. Acad. Sci. U.S.A 96, 12703-12707 (1999).
91. Kenna, T. J. et al. Targeting Antigen to Diverse APCs Inactivates Memory CD8+ T Cells without Eliciting Tissue-Destructive Effector Function. J. Immunol. 184, 598-606 (2010).
92. Leiter, E. H. Multiple low-dose streptozotocin-induced hyperglycemia and insulitis in C57BL mice: Influence of inbred background, sex, and thymus. Proc. Natl. Acad. Sci. U.S.A. 79, 630-634 (1982).
93. Jabara, H. H. et al. A missense mutation in TFRC, encoding transferrin receptor 1, causes combined immunodeficiency. Nat. Genet. 48, 74-78 (2015).
94. Badran, Y. R. et al. Human RELA haploinsufficiency results in autosomal-dominant chronic mucocutaneous ulceration. J. Exp. Med. 214, 1937-1947 (2017).
95. Zehn, D. & Wherry, E. J. Immune Memory and Exhaustion: Clinically Relevant Lessons from the LCMV Model. in Crossroads Between Innate and Adaptive Immunity V (eds. Schoenberger, S. P., Katsikis, P. D. & Pulendran, B.) 850, 137-152 (Springer International Publishing, 2015).
96. Althaus, C. L., Ganusov, V. V. & Boer, R. J. D. Dynamics of CD8+ T Cell Responses during Acute and Chronic Lymphocytic Choriomeningitis Virus Infection. J. Immunol. 179, 2944-2951 (2007).
97. Wherry, E. J., Blattman, J. N., Murali-Krishna, K., van der Most, R. & Ahmed, R. Viral Persistence Alters CD8 T-Cell Immunodominance and Tissue Distribution and Results in Distinct Stages of Functional Impairment. J. Virol. 77, 4911-4927 (2003).
98. Hu, Y. et al. Scramblase TMEM16F terminates T cell receptor signaling to restrict T cell exhaustion. J. Exp. Med. 213, 2759-2772 (2016).
99. Fahey, L. M. et al. Viral persistence redirects CD4 T cell differentiation toward T follicular helper cells. J. Exp. Med. 208, 987-999 (2011).
100. Barnett, B. E. et al. B cell intrinsic T-bet expression is required to control chronic viral infection. J. Immunol. Baltim. Md 1950 197, 1017-1022 (2016).
101. Oldstone, M. B. A. et al. Lymphocytic choriomeningitis virus Clone 13 infection causes either persistence or acute death dependent on IFN-1, cytotoxic T lymphocytes (CTLs), and host genetics. Proc. Natl. Acad. Sci. 115, E7814-E7823 (2018).
102. Charan, J. & Kantharia, N. D. How to calculate sample size in animal studies? J. Pharmacol. Pharmacother. 4, 303 (2013).

### Example 2: Leucine-Rich Repeat Containing 8A (LRRC8A) is Essential for the Re-Activation of CD4+ T Cells and the Development of Acute Graft Versus Host Disease

Non-hematopoietic antigen presenting cells (APCs) in inflamed tissues acquire the capacity to re-activate CD4+ T cells, culminating in organ damage. However, the mechanisms influencing secondary T cell activation by non-hematopoietic APCs are incompletely understood. Here, the inventors identify the transmembrane protein Leucine-Rich Repeat-Containing Protein 8A (LRRC8A) as essential for the reactivation of antigen-experienced T cells. The inventors show that the functions of LRRC8A in T cell activation are independent of its known function as a pore-forming subunit in the volume-regulated anion channel (VRAC). The inventors found that LRRC8A colocalizes with the T cell receptor (TCR) and enhances TCR-driven gene expression. The selective deletion of LRRC8A in CD4+ T cells impairs mitochondrial biogenesis and respiration, glycolysis, proliferation, survival, and cytokine secretion during the secondary response to antigen. Conditional deletion of LRRC8A from donor CD4+ T cells attenuates acute graft versus host disease arising from mismatched minor histocompatibility antigens presented by non-hematopoietic APCs. These findings demonstrate the biologic relevance of LRRC8A in T cell activation and suggest its potential as a therapeutic target in acute GVHD.

CD4+ T cells have distinct requirements for primary and secondary activation. During primary activation, naïve CD4+ T cells respond to hematopoietic antigen presenting cells (APCs) that have constitutive expression of major histocompatibility antigen complex (MHC) class II molecules (1). The secondary activation of antigen-experienced CD4+ T cells requires less antigen and costimulation than primary activation (2, 3). During inflammation, non-hematopoietic APCs, such as fibroblasts, keratinocytes, and epithelial cells, upregulate MHC class II and acquire the capacity to activate CD4+ T cells. Despite lower expression of costimulatory signals compared to their hematopoietic counterparts (1), non-hematopoietic APCs activate antigen-experienced T cells in tissues, culminating in end-organ damage (4-8). However, the signals influencing secondary T cell activation by stromal APCs remain incompletely understood.

The inventors showed herein that early T cell development requires the transmembrane protein Leucine-Rich Repeat-Containing Protein 8A (LRRC8A) (9). Constitutive deficiency of LRRC8A inhibits thymocyte development at the double negative (DN) 2 to DN3 stages, resulting in severely impaired T cell development (9). Lrrc8a−/− thymocytes were more susceptible to apoptosis due to reduced signaling through the phosphoinositide 3-kinase (PI3K)-AKT pathway (9). LRRC8A is known to have another function as the essential pore-forming subunit of the volume-regulated anion channel (VRAC) in the plasma membrane (10-12). In response to hypotonic stress, the VRAC channel releases anions and organic osmolytes, permitting water efflux and normalization of cellular volume (13). Recent studies identified cellular functions dependent on LRRC8A-mediated volume regulation, including spermatid development amidst osmolar changes in the epididymis, pancreatic β-cells undergoing glucose-induced osmotic swelling, and astrocytes exposed to ischemia (14-16). We subsequently demonstrated that the contribution of LRRC8A to T cell development is independent of its channel activity (17). However, the role of LRRC8A in mature T cell function remains unknown: the impaired function of residual T cells in Lrrc8a−/− mice may result from either severely defective thymocyte development or an intrinsic role for LRRC8A in peripheral T cell function.

To address this, the inventors generated a Cd4-Cre Lrrc8afl/fl mouse model in which LRRC8A is conditionally deleted at the very late DN4 stage of thymocyte development. This model bypasses the developmental checkpoint at the DN2 thymocyte stage requiring LRRC8A expression, thereby preserving T cell development. The inventors found that LRRC8A colocalizes with the T cell receptor (TCR) and is necessary for optimal signaling downstream of TCR ligation. Selective deletion of LRRC8A in CD4+ T cells impairs T cell metabolism and effector functions during the secondary response to antigen. To test the biologic relevance of LRRC8A in T cell-driven diseases, the inventors investigated whether conditional deletion of LRRC8A in donor T cells ameliorates CD4+-driven acute GVHD arising from mismatched minor histocompatibility antigens.

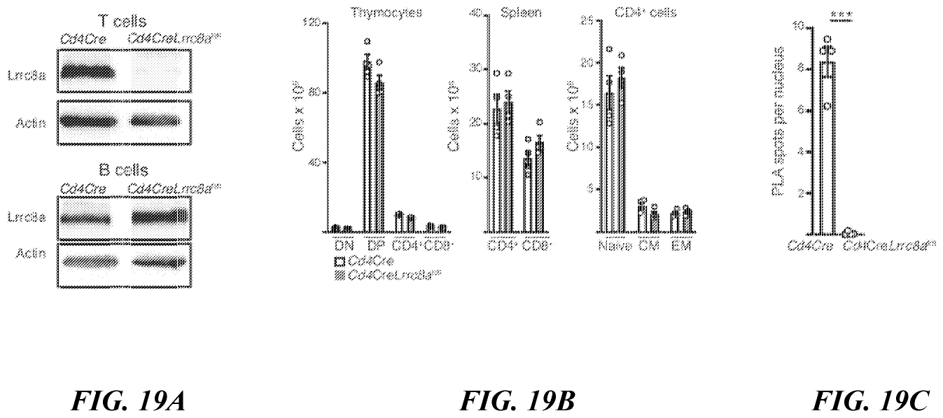

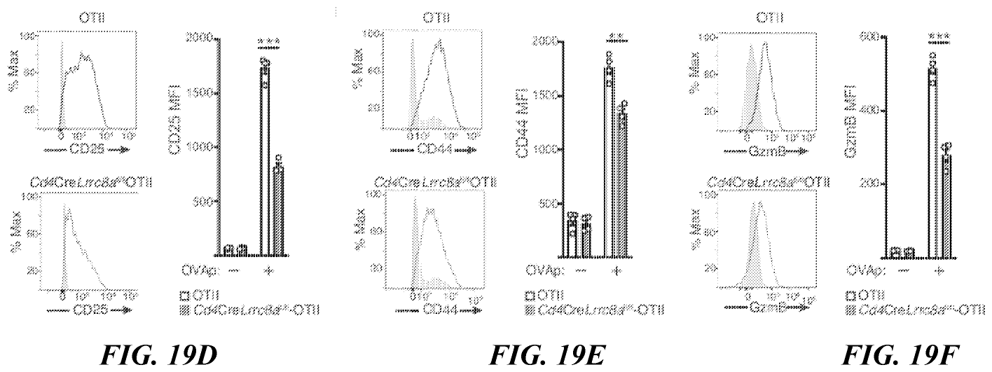

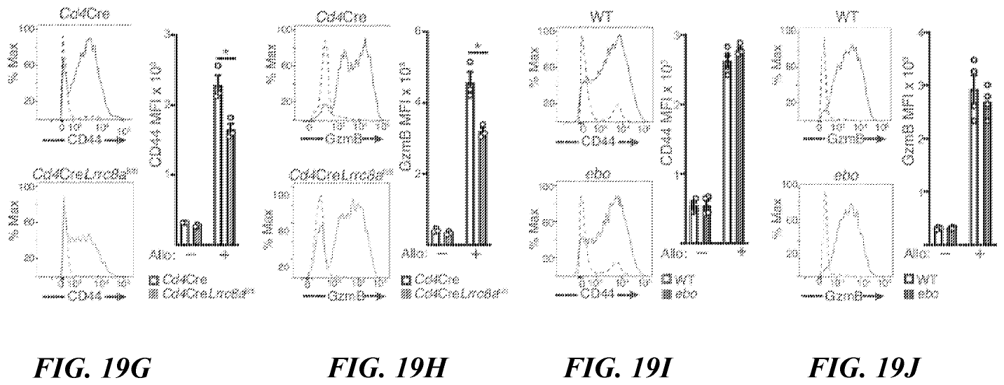

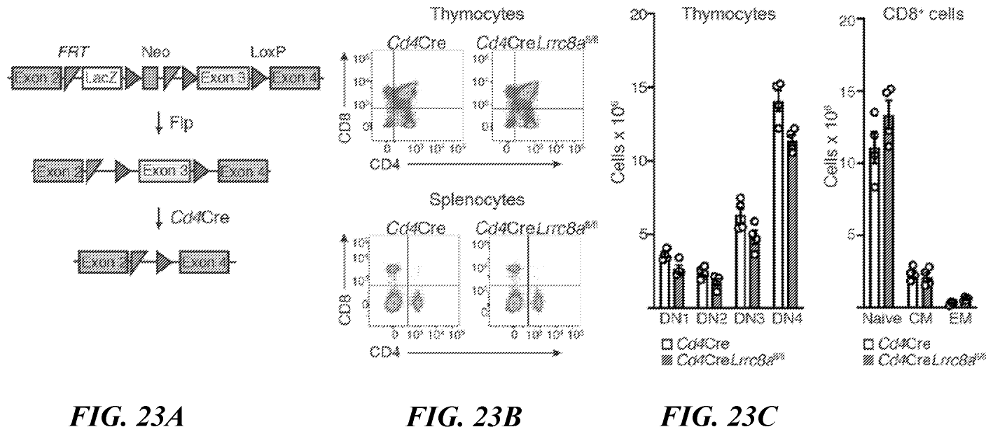

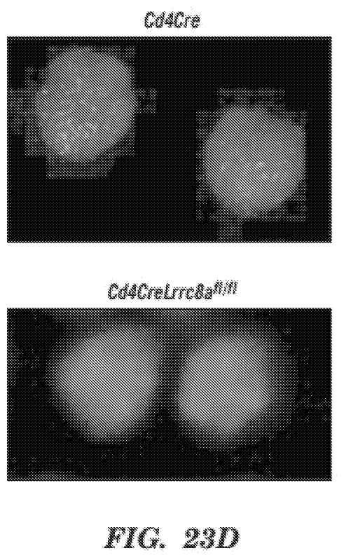

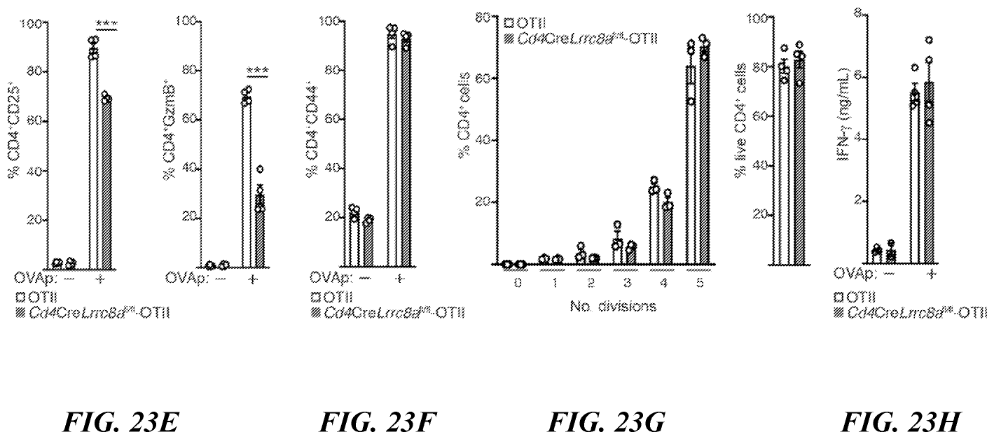

Since Lrrc8a−/− mice have multi-systemic abnormalities, perinatal mortality, and a severe block in the transition between the DN2 and DN3 stages of thymocyte development (9), the inventors generated Cd4-Cre Lrrc8afl/fl mice in which Lrrc8a is selectively deleted in very late DN4 thymocytes (FIG. 23A). LRRC8A protein expression was undetectable in CD3+ T cells, but intact in B cells, from spleens of Cd4-CreLrrcafl/fl mice, confirming selective deletion of LRRC8A in T cells (FIG. 19A). Cd4-Cre Lrrc8afl/fl mice had normal numbers of thymocytes, CD4+ T cells, and CD8+ T cells (FIG. 19B, FIG. 23B, FIG. 23C). Naïve and memory splenic CD4+ and CD8+ T cells were also intact in Cd4-Cre Lrrc8afl/fl mice (FIG. 19B and FIG. 23C). LRRC8A is thus dispensable for T cell development after the DN4 stage of T cell development.

Since Lrrc8a−/− mice have multi-systemic abnormalities, perinatal mortality, and a severe block in the transition between the DN2 and DN3 stages of thymocyte development (9), the inventors generated Cd4-Cre Lrrc8afl/fl mice in which Lrrc8a is selectively deleted in very late DN4 thymocytes (FIG. 23A). LRRC8A protein expression was undetectable in CD3+ T cells, but intact in B cells, from spleens of Cd4-CreLrrcafl/fl mice, confirming selective deletion of LRRC8A in T cells (FIG. 19A). Cd4-Cre Lrrc8afl/fl mice had normal numbers of thymocytes, CD4+ T cells, and CD8+ T cells (FIG. 19B, FIG. 23B, FIG. 23C). Naïve and memory splenic CD4+ and CD8+ T cells were also intact in Cd4-Cre Lrrc8afl/fl mice (FIG. 19B, FIG. 23C). LRRC8A is thus dispensable for T cell development after the DN4 stage of T cell development.

The inventors next assessed the role of LRRC8A in peripheral T cell activation. The inventors and others have shown that LRRC8A is needed for intact signaling through the phosphoinositide 3 kinase (PI3K)-AKT pathway (9, 18, 19), a pathway critical for TCR signaling. To determine if LRRC8A colocalizes with the TCR, the inventors utilized proximal ligation assays in which target proteins are tagged with oligonucleotide-labeled antibodies (20). The subsequent addition of DNA ligase, DNA polymerase, and fluorescent oligonucleotides generates circular DNA templates only between tagged proteins in close proximity (<40 nm) (20). The inventors found that LRRC8A co-localizes with CD3ε, demonstrated by fluorescent DNA templates detected on CD4+ T cells from WT, but not Cd4-Cre Lrrc8afl/fl mice (FIG. 19C, FIG. 23D). To determine the role of LRRC8A in antigen-driven TCR activation, the inventors bred Cd4-Cre Lrrc8afl/fl mice on the OTII transgenic background, thereby generating conditional LRRC8A-deficient CD4+ T cells bearing the T cell receptor specific for ovalbumin (OVA) peptide 323-339. After primary activation with OVA peptide presented by splenic B cells, Cd4-Cre Lrrc8afl/fl-OTII CD4+ T cells had lower expression of the activation markers CD25 and CD44 compared to controls (FIG. 19D and FIG. 19E). Granzyme B, an effector protein expressed on CD4+ T cells after prolonged antigen exposure (21), was reduced in activated Cd4-Cre Lrrc8afl/fl-OTII CD4+ T cells compared to controls (FIG. 19F). Additionally, Cd4-Cre Lrrc8afl/fl-OTII CD4+ T cells undergoing primary activation had lower percentages of CD4+CD25+ and CD4+granzyme B+ cells compared to controls (FIG. 23E). However, Cd4-Cre Lrrc8afl/fl-OTII CD4+ T cells did not exhibit globally defective primary activation. The percentages of CD4+CD44+ cells, proliferation, survival, and IFN-γ secretion were comparable between stimulated Cd4-Cre Lrrc8afl/fl-OTII and control OTII CD4+ T cells (FIG. 23F, FIG. 23G, FIG. 23H). As a complementary approach, the inventors assessed activation of WT or Cd4-Cre Lrrc8afl/fl CD4+ T cells on the C57BL/6J background in a mixed lymphocyte reaction with mismatched MHC class I and class II target cells on the FVB/N (Balb/c substrain) background. Cd4-Cre Lrrc8afl/fl CD4+ T cells exhibited reduced expression of CD25, CD44, and granzyme B, despite undergoing comparable proliferation relative to controls (FIG. 19 G and FIG. 19H, FIG. 24A, FIG. 24B). These findings demonstrate that LRRC8A enhances the optimal primary activation of CD4+ T cells, although it is dispensable for T cell proliferation and IFN-γ secretion.

The inventors next determined whether VRAC channel activity is necessary for mature T cell activation. Due to a spontaneous deletion of two nucleotides in Lrrc8a, ébouriffé (ebo) mice express an LRRC8A mutant lacking 15 C-terminal leucine rich repeats (17). Despite protein expression comparable to full-length LRRC8A on control cells, the ebo LRRC8A mutant has negligible channel activity (17). The inventors previously showed that lymphocyte development was intact in ebo mice, indicating that LRRC8A has a function in immune cells independent of its channel activity (17). Since the ebo mutant occurred spontaneously on the FVB background, the inventors assessed CD44 and granzyme B expression in ebo CD4+ T cells in a mixed lymphocyte reaction with C57BL/6J target cells. Activated ebo CD4+ T cells exhibited normal expression of CD25, CD44 and granzyme B compared to controls, thereby demonstrating that the channel activity of LRRC8A is dispensable for T cell activation under iso-osmotic conditions (FIG. 19I, FIG. 19J, FIG. 24C, FIG. 24D).

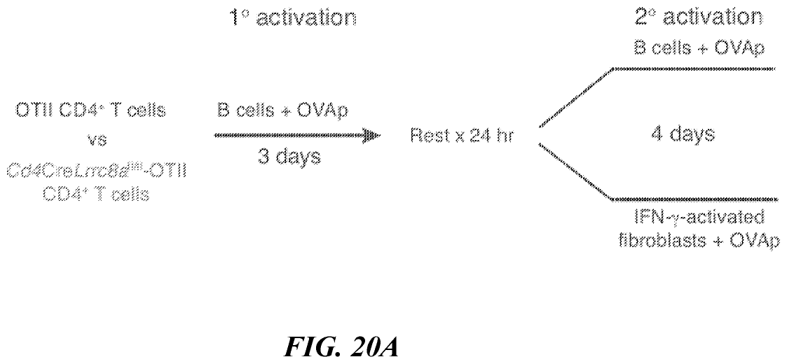

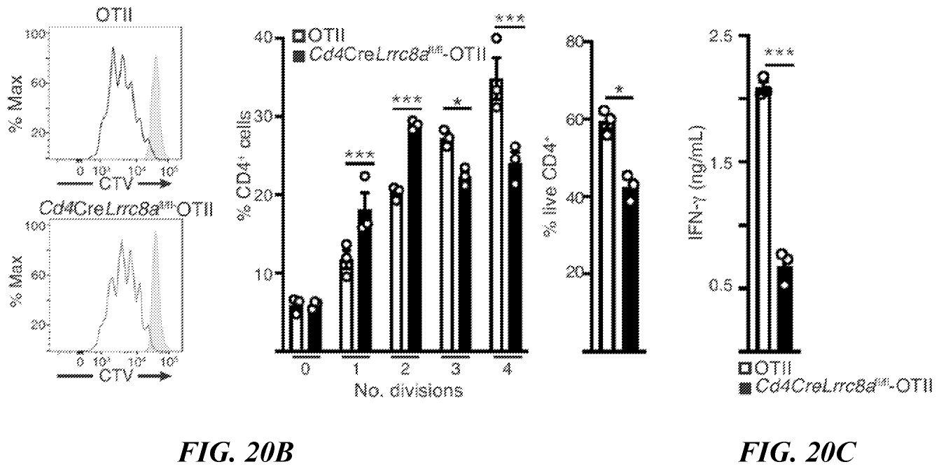

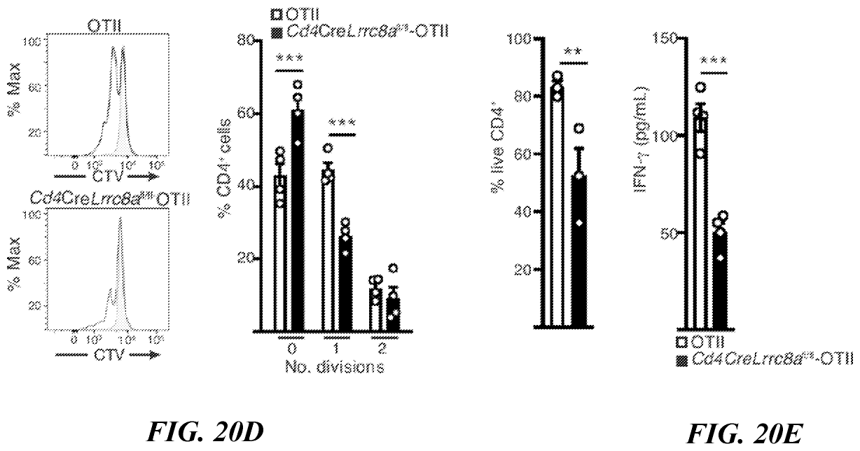

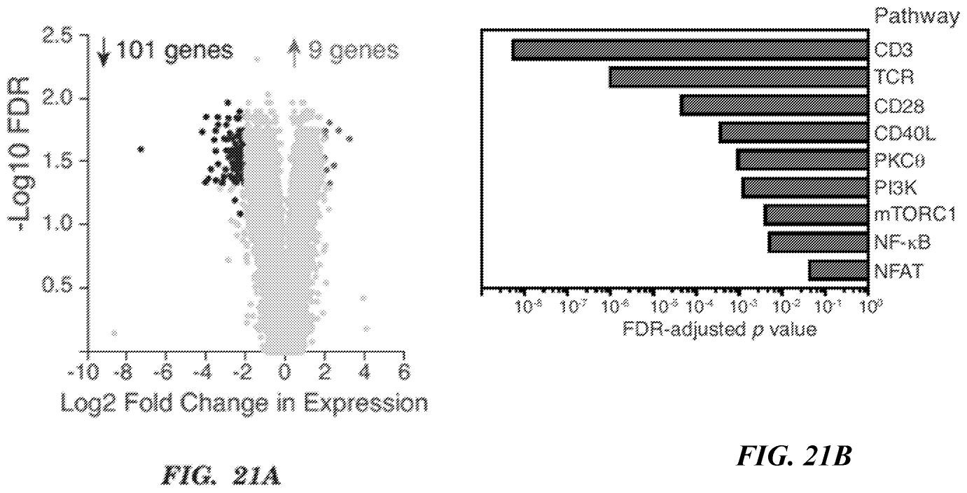

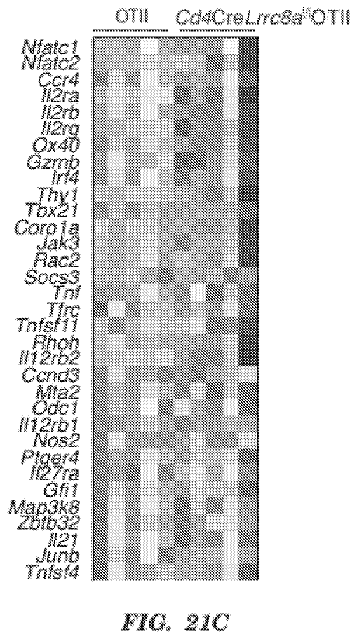

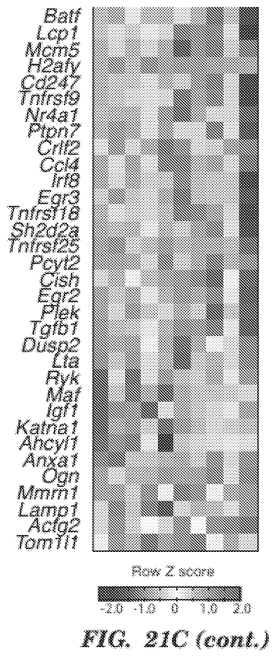

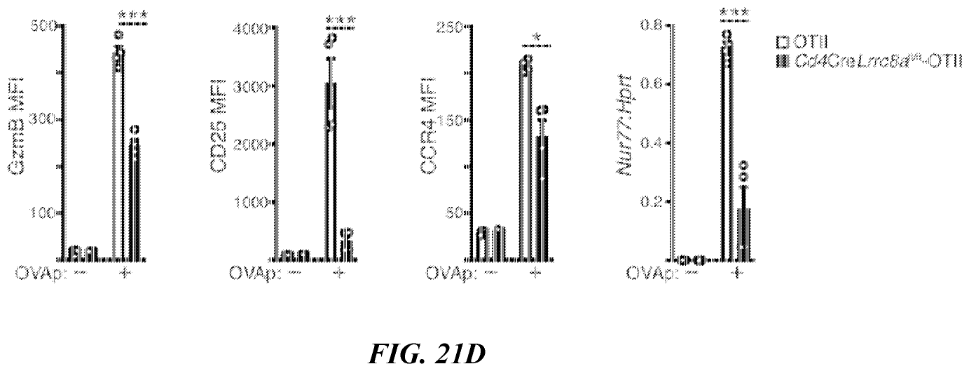

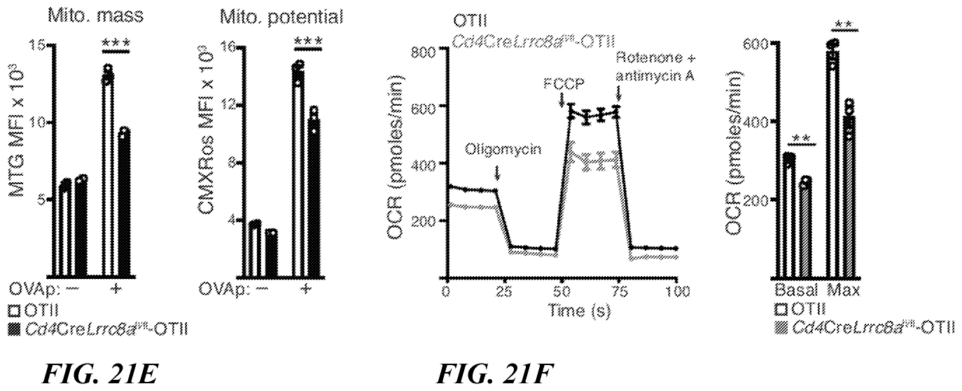

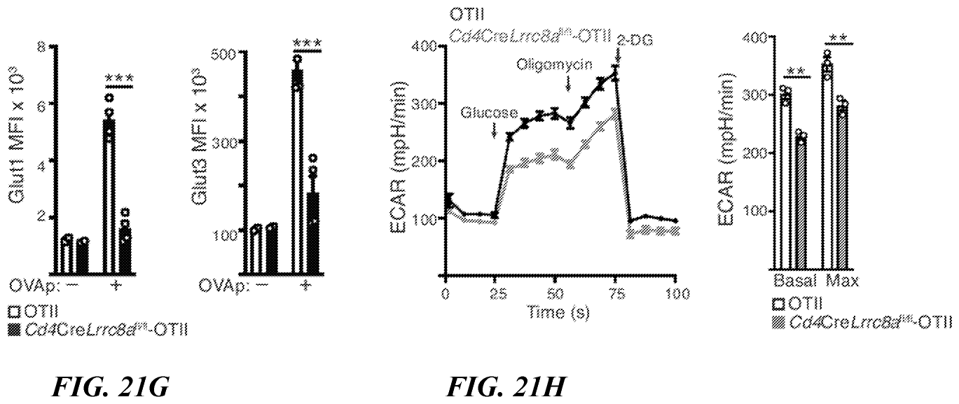

Antigen-experienced T cells in tissues undergo secondary activation driven by hematopoietic and non-hematopoietic APCs (2, 3). To study the role of LRRC8A in secondary T cell activation, WT-OTII or Cd4-Cre Lrrc8afl/fl-OTII CD4+ T cells were stimulated with OVA peptide, rested, and reactivated with OVA peptide presented by either WT splenic B cells or IFN-γ-treated fibroblasts (FIG. 20A). Antigen-primed Cd4-Cre Lrrc8afl/fl-OTII CD4+ T cells had defective proliferation, reduced cell survival, and IFN-γ secretion after secondary activation with OVA peptide presented by splenic B cells and by fibroblasts (FIG. 20B, FIG. 20E). The biologic relevance of CD4+ T cell activation by non-hematopoietic APCs has been highlighted in studies showing that stromal APCs are necessary and sufficient for CD4+ T cell-mediated acute GVHD due to mismatched minor histocompatibility antigens (4, 5, 22). Furthermore, multiple studies have shown that ablation of recipient hematopoietic APCs fails to prevent acute GVHD and can worsen disease severity (5, 22-24). Thus, the inventors investigated LRRC8A-dependent changes in CD4+ T cell gene expression during reactivation by antigen-presenting fibroblasts. The inventors performed whole transcriptome analysis of WT-OTII or Cd4-Cre Lrrc8afl/fl-OTII CD4+ T cells after secondary activation with OVA peptide-presenting fibroblasts. Using a false discovery rate of 5%, the inventors identified 101 downregulated and 9 upregulated genes with at least a two-fold difference in expression in re-stimulated Cd4-Cre Lrrc8afl/fl-OTII CD4+ T cells compared to controls (FIG. 21A and Exhibit A). Ingenuity® pathway analysis revealed significant enrichment of downregulated genes in pathways important for T cell activation, including CD3, TCR, CD28, CD40L, PKCθ, PI3K, mTORC1, NF-kB, and NF-AT (FIG. 21B). Within the CD3 signaling pathway, 83% of the genes had reduced expression in re-stimulated Cd4-Cre Lrrc8afl/fl-OTII CD4+ T cells compared to controls (FIG. 21C). These genes encoded proteins important for the chemotaxis and function of memory/effector T cell populations (CCR4 and granzyme B), cytokine receptors (receptors for IL2, IL12, IL27), and transcription factors (NFAT, T-bet, BATF, IRF4) (FIG. 21C). The inventors confirmed that protein expression of CCR4, granzyme B, and CD25/IL2Ra were reduced in reactivated Cd4-Cre Lrrc8afl/fl-OTII CD4+ T cells (FIG. 21D). As these data suggest that selective deletion of LRRC8A decreases TCR signaling during secondary activation, the inventors assessed expression of Nur77, an immediate early response gene whose expression reflects the strength of TCR signaling (25). Cd4-Cre Lrrc8afl/fl-OTII CD4+ T cells exhibited reduced expression of Nur77 after secondary activation of fibroblasts (FIG. 21D), concordant with the transcriptional profile of decreased TCR activation. Collectively, these findings demonstrate that LRRC8A is essential for the re-activation of antigen-experienced CD4+ T cells. This contrasts with the more limited role of LRRC8A during primary activation.

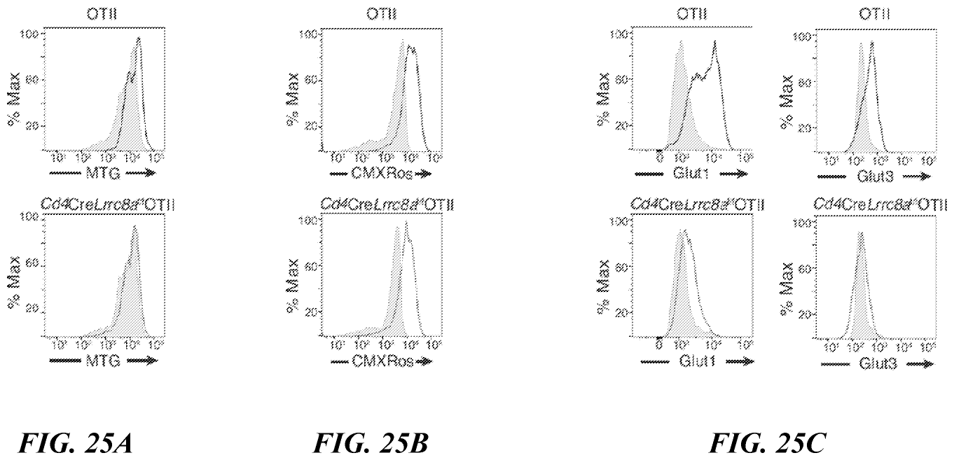

TCR signaling induces metabolic reprogramming, including increased mitochondrial mass, membrane potential, and respiration (26). After secondary activation with antigen-presenting fibroblasts, Cd4-Cre Lrrc8afl/fl-OTII CD4+ T cells had reduced mitochondrial mass and membrane potential compared to controls (FIG. 21E, FIG. 25A, FIG. 25B). The inventors assessed mitochondrial respiration by measuring the oxygen consumption rate (OCR) in WT-OTII and Cd4-Cre Lrrc8afl/fl-OTII CD4+ T cells after secondary activation. In this assay, oligomycin inhibits the ATP synthase and reveals basal cellular respiration (27). Carbonyl cyanide-4(trifluoromethoxy) phenylhydrazone (FCCP) permeabilizes the inner mitochondrial membrane, allowing unrestricted proton influx to maximize mitochondrial respiration (27). Rotenone and antimycin A inhibit mitochondrial respiration, revealing the rate of non-mitochondrial oxygen consumption (27). Conditional deletion of LRRC8A in CD4+ T cells impaired basal and maximal respiration during the secondary response to OVA peptide, reflecting reduced mitochondrial respiration (FIG. 21F).

While oxidative phosphorylation is the most efficient pathway for ATP production, glycolysis generates substrates for the synthesis of effector proteins (26). Through PI3K and mTOR signaling, TCR stimulation upregulates the glucose transporters GLUT1 and GLUT3 (28, 29). After secondary activation, Cd4-Cre Lrrc8afl/fl-OTII CD4+ T cells exhibited decreased expression of GLUT1 and GLUT3 (FIG. 21G, FIG. 25C), consistent with reduced gene expression in the PI3K pathway (FIG. 21B). Since the extracellular acidification rate (ECAR) is an established indicator of glycolysis (30), the inventors measured ECAR from CD4+ T cells after secondary activation with antigen-presenting fibroblasts. Re-activated CD4+ T cells were exposed to saturating concentrations of glucose, followed by oligomycin to inhibit mitochondrial respiration and maximize glycolysis (30). The subsequent addition of 2-deoxy-d-glucose (2-DG) inhibits glycolysis, revealing the level of non-glycolytic acidification. After secondary activation, Cd4-Cre Lrrc8afl/fl-OTII CD4+ T cells exhibited reduced ECAR indicative of impaired glycolysis (FIG. 21H). Collectively, these findings demonstrate that selective deletion of LRRC8A in CD4+ T cells impairs mitochondrial respiration and glycolysis, thus inhibiting the metabolic reprogramming of CD4+ T cells during the secondary response to antigen.

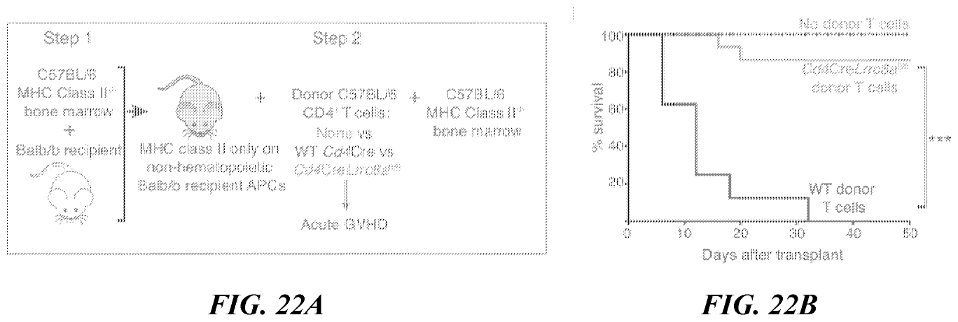

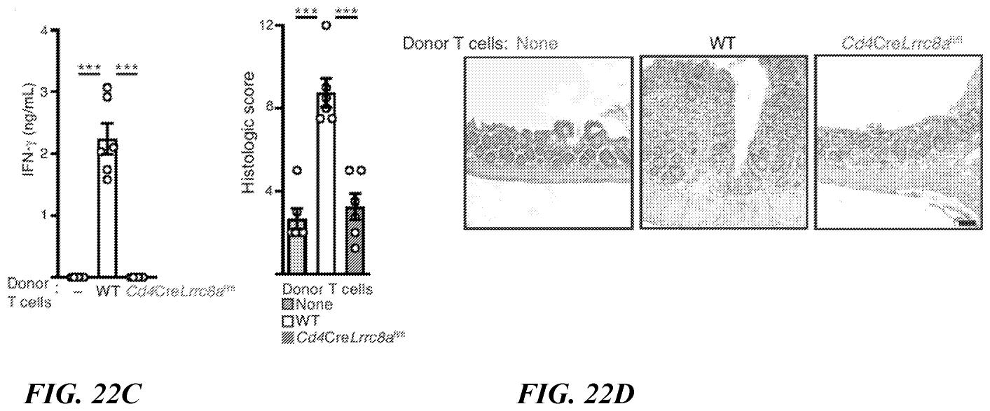

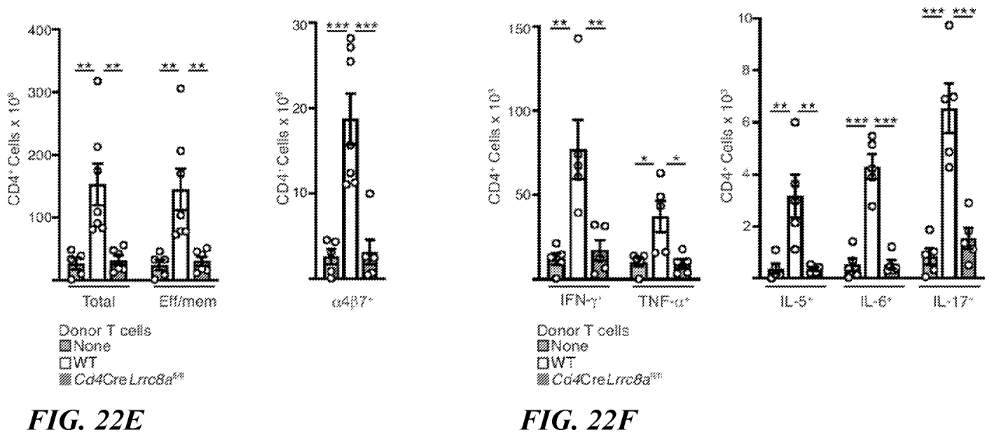

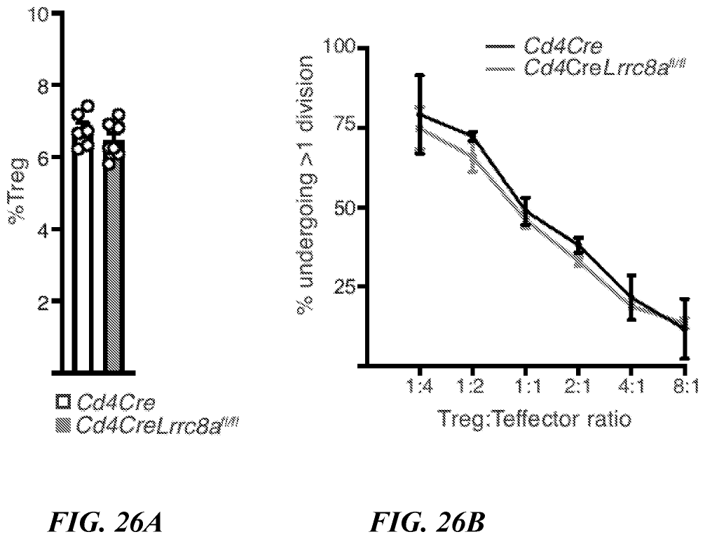

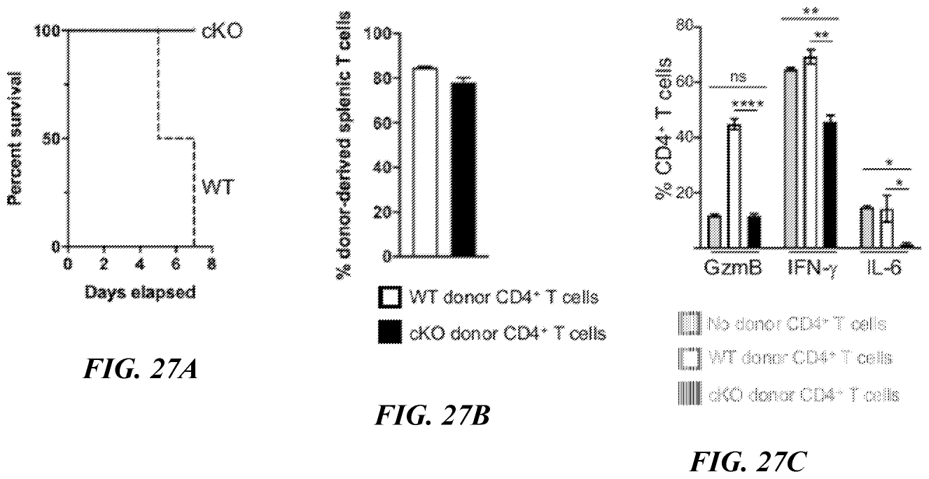

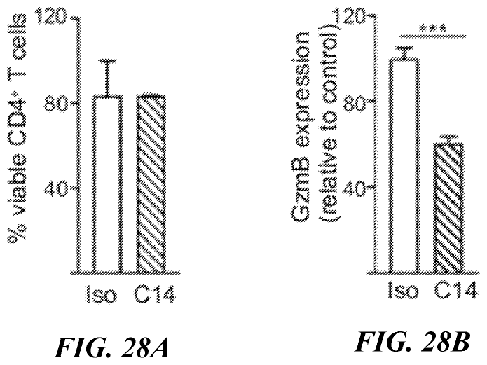

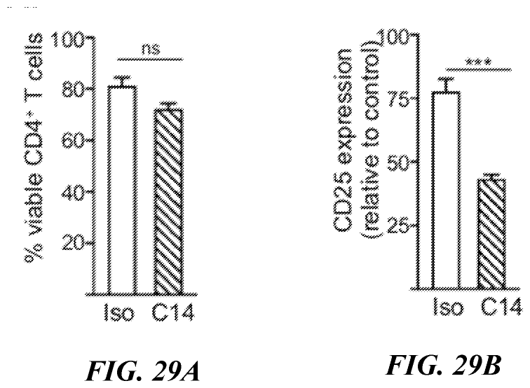

The reactivation of antigen-experienced T cells by stromal APCs drives organ damage in acute GVHD (4, 5, 22). Thus, the inventors tested the contribution of LRRC8A on donor T cells using a well-established model in which C57BL/6J donor CD4+ T cells recognize mismatched minor histocompatibility antigens presented by recipient Balb/b stromal APCs (4, 5). The C57BL/6J strain shares major, but differs in minor, histocompatibility antigens with the Balb/b strain. To generate chimeric recipients with MHC class II expression solely on non-hematopoietic cells, the inventors reconstituted irradiated Balb/b recipients with T cell-depleted bone marrow from MHC class II-deficient mice. After reconstitution, the inventors induced acute GVHD in irradiated chimeric recipients by transplanting WT-Cd4-Cre or Cd4-Cre Lrrc8afl/fl CD4+ donor T cells with T cell-depleted, MHC class II-deficient bone marrow (FIG. 22A). Similar to prior studies, no recipients of donor WT-Cd4-Cre CD4+ T cells survived beyond 32 days post-transplantation (4, 5). In contrast, 78% of those who received donor Cd4-Cre Lrrc8afl/fl CD4+ T cells remained alive at 50 days post-transplantation (FIG. 22B). Recipients of Cd4-Cre Lrrc8afl/fl CD4+ T cells had undetectable serum IFN-γ at seven days post-transplantation and less weight loss, consistent with attenuated GVHD (FIG. 22C). Histopathologic analysis of the colon from recipients of WT-Cd4-Cre CD4+ T cells revealed a dense lymphocytic infiltrate in the lamina propria and crypt destruction, neither of which was seen in recipients of conditional LRRC8A-deficient CD4+ T cells (FIG. 22D). The colonic lamina propria in recipients of conditional LRRC8A-deficient T cells contained fewer CD4+CD62loCD44hi effector/effector memory T cells and lower expression of the α4β7 integrin LPAM (FIG. 22E), which is upregulated on alloreactive T cells during GVHD (31). Compared to controls, Cd4-Cre Lrrc8afl/fl CD4+ T cells in the lamina propria also expressed lower levels of cytokines important for the pathogenesis of acute GVHD: IFN-γ, TNF-α, IL-5, IL-6, and IL-17 (FIG. 22F). The numbers of regulatory T cells in the bone marrow of Cd4-Cre Lrrc8afl/fl and control donors were comparable and the suppressor function of Cd4-Cre Lrrc8afl/fl regulatory T cells was intact (FIG. 26), thus confirming that differences in GVHD severity was not due to differences in regulatory T cell number or function. These data show that LRRC8A is essential for CD4+ T cell-driven acute GVHD arising from mismatched minor histocompatibility antigens presented by non-hematopoietic APCs.

Here, the inventors identify a new role for LRRC8A in the re-activation of antigen-experienced CD4+ T cells, a central process in T cell-driven diseases. In contrast, the inventors found a limited role for LRRC8A during primary activation. Analogously, the phosphatase PTP-PEST enhances the secondary activation of T cells by promoting the formation of T cell homoaggregates and enhancing TCR signaling, but has no contribution to primary T cell activation (32). Compared to naïve T cells, antigen-experienced T cells have an increased density of TCR oligomers, which correlates with heightened sensitivity to antigen (3). The inventors found that LRRC8A colocalizes with the TCR and promotes transcriptional and metabolic profiles characteristic of TCR activation. Collectively, the inventors' findings show that selective deletion of LRRC8A in T cells undergoing reactivation impairs proliferation, cellular metabolism, and effector functions both in vitro and in vivo. These findings clearly show the therapeutic relevance of LRRC8A as a target for the treatment of acute GVHD.

Methods

Mice. Lac8a^{tm2a(EUCOMM)Hmgu} mice were bred with Flp-recombinase deleter mice to remove the LacZ and neomycin resistance cassette, followed by breeding with Cre-recombinase delete mice to remove exon 3 of LRRC8A (FIG. 23A). The ébouriffé (ebo) mice have been previously described (1). The following strains were purchased from the Jackson Laboratory: OT-II (B6.Cg-Tg(TcraTcrb)425Cbn/J; stock number 004194), Cd4-Cre (Tg(Cd4-cre)1Cwi/BfluJ; H-2D^{b}, stock number 017336), WT C57BL/6J (H-2D^{b}, stock number 017336), BALB/B (H-2D^{b}, stock number 001952), MHC II-deficient H2^{dlAb1-Ea} (stock number 003584), and wild-type FVB/NJ (H-2D^{q}, stock 001800) mice. All procedures were performed within the guidelines of the Animal Care and Use Committee of Boston Children's Hospital and the study was approved by the Boston Children's Hospital Institutional Animal Care and Use Committee.

Antibodies and mitochondrial stains. Antibodies to CD4 (GK1.5), CD8 (53-6.7), CD44 (IM7), CD25 (PC61), CCR4 (2G12), CD62L (MEL-14), Granzyme B (QA16A02), TNF-α (MP6-XT22), IL-5 (TRFK5) and IL-6 (MP5-20F3) were purchased from Biolegend. Antibodies to GLUT1 (EPR3915) and GLUT3 (ab136180) were purchased from Abcam. Antibodies to LPAM-1 (DATK32), IFN-γ (XMG1.2), IL-17A (eBio7B7), and β-actin (AC-15) were purchased from ThermoFisher Scientific. The c-terminal anti-LRRC8A antibody were previously described (1). Cells were stained with. 100 nm MitoTracker Green FM (M7514) and 200 nM MitoTracker Red CMXRos (M7512), both from ThermoFisher Scientific, per the manufacturer's guidelines.

Flow Cytometry. Unless otherwise stated, cells were stained for viability using eBioscience™ Fixable Viability Dye eFluor™ 506 (6ThermoFisher Scientific). Non-specific interactions were blocked using TruStain FcX™ (anti-mouse CD16/32) antibody (Biolegend). For intracellular staining, cells were fixed and permeabilized using Fixation/Permeabilization Solution Kit (BD Biosciences). For proliferation, cells were stained with CellTrace™ Violet (ThermoFisher Scientific).

Cell cultures and activation. Primary activation. Single cell splenocyte suspensions were prepared from the murine genotypes indicated in the figure legends and stimulated with 7.5 μg/mL OVA₃₂₃₋₃₃₉ (RP10610, Genscript) for four days. Secondary activation. At the end of the four-day primary activation, CD4⁺ T cells were purified using magnetic negative selection and rested overnight before secondary activation with splenic B cells or IFN-γ-activated alveolar fibroblasts and 1 μg/mL OVA₃₂₃₋₃₃₉ for three days. Splenic B cells were purified using magnetic negative selection (130-091-041, Miltenyi Biotec). C57BL/6J alveolar fibroblasts were activated for five days with 100 ng/mL mIFN-γ (485-MI, R&D Systems) to induce MHC class II upregulation. Mixed Lymphocyte Reaction. Splenic CD4⁺ T cells were purified from Cd4-CreTg, Cd4-Cre Lrrc8a^{fl/fl}, ebo, and FVB/NJ mice splenocytes using magnetic negative selection (130-104-454, Miltenyi Biotec) and stained with CellTrace™ Violet (ThermoFisher Scientific). Stimulator C57BL/6J or FVB/NJ splenocytes were pulsed with 50 μg/mL mitomycin C (M4287, Sigma-Aldrich) in PBS for 20 minutes, then washed twice using RPMI medium supplemented with 10% fetal calf serum, glutamine, penicillin-streptomycin, and β-mercaptoethanol. Responders and stimulators, as indicated by the figure legends, were then cocultured for 5 days.

Immunoblotting. Splenic T and B cells were purified by magnetic negative selection (130-095-130 and 130-049-801, Miltenyi Biotec). Lysates were prepared in RIPA lysis and extreaction buffer (ThermoFisher Scientific) supplemented with LDS Sample Buffer (ThermoFisher Scientific) and 2-mercaptoethanol (M6250, Sigma Aldrich). Protein samples were resolved by SDS-PAGE (Bio-Rad), transferred onto a PVDF membrane (ThermoFisher Scientific), followed by immunoblotting with antibodies as indicated in the figure legend. Immunoblots were imaged used the iBright Imaging System (ThermoFisher Scientific).

Proximity Ligation Assay. Briefly, Cd4-CreTg and Cd4-Cre Lrrc8a^{f/f} CD4⁺ T cells were plated over Poly-L-Lysine (P4707, Sigma Aldrich) coated coverslips. The proximity ligation assay was performed per the manufacturer's guidelines using the following reagents: Duolink In Situ Detection Reagents Red (DUO92008), Duolink In Situ PLA Probe Anti-Rabbit PLUS (DU092002), Duolink In Situ PLA Probe Anti-Mouse MINUS (DU092004), and Duolink In Situ Mounting Medium with DAPI (DU082040), all from Sigma Aldrich. Antibodies used were: anti-CD3e (GT0013; host species: mouse) and anti-LRRC8A (host species: rabbit; described in (1)).

Cytometric Bead Array. Concentrations of IL2 and IFN-γ were measured using Mouse IL2 Flex Set (558297, BD Biosciences), Mouse IFN-γ Flex Set (558296, BD Biosciences) and Mouse/Rat Soluble Protein Master Buffer Kit (558267, BD Biosciences) according to the manufacturer's protocol.

Whole transcriptome sequencing. After secondary stimulation with OVA peptide-presenting fibroblasts, CD4⁺ T cells were purified by magnetic positive selection (130-117-043, Miltenyi Biotec). RNA was isolated from the cells using RNeasy Micro Kit (74004, Qiagen). cDNA was then synthesized from 10 ng of total RNA using SuperScript™ VILO™ cDNA Synthesis Kit (11754050, ThermoFisher Scientific). Barcoded libraries were prepared using the Ion AmpliSeq Transcriptome Mouse Gene Expression Kit as per the manufacturer's protocol and sequenced using an Ion S5™ system. Differential gene expression analysis was performed using the ampliSeqRNA plugin (ThermoFisher). Pathway analysis was done using Ingenuity Pathway Analysis (Qiagen).

Extracellular flux analysis. Mitochondrial respiration was measured with the Seahorse XFp Cell Mito Stress Test Kit (103010-100, Agilent Technologies) performed on an Agilent Seahorse XFp analyzer (Agilent Technologies) per manufacturer's guidelines. Cells were plated at 100k cells/well of an Agilent Seahorse XFp Cell Culture Miniplate. The Agilent Seahorse XFp Extracellular Flux Cartridge was loaded as follows: 20 μL of 10 μM Oligomycin in port A, 22 μL of 40 μM FCCP in port B, and 25 μL of 5 μM Rotenone/Antimycin A in port C.

Glycolysis was measured with the Seahorse XFp Glycolysis Stress Test Kit (103017-100, Agilent Technologies) performed on an Agilent Seahorse XFp analyzer (Agilent Technologies) per the manufacturer's guidelines. Cells were plated at 100k cells/well of an Agilent Seahorse XFp Cell Culture Miniplate. The Agilent Seahorse XFp Extracellular Flux Cartridge was loaded as follows: 20 μL of 100 mM Glucose in port A, 22 μL of 10 μM Oligomycin in port B, and 25 μL of 500 mM 2-deoxyglucose in port C.

Graft versus host disease (GVHD). Mice were transplanted as described previously (2). BALB/B mice were exposed to 900 cGy of total body irradiation, split in two doses administered three hours apart. Lethally irradiated mice were transplanted with 5×10⁶ bone marrow cells to generate chimeras. Engraftment was assessed 5 weeks after the first transplant using B2 m^{b}-FITC conjugated antibody (Santa Cruz), since C57/B6 mice express B2 m^{b}, while BALB/B mice express B2ma. Mice that had >98% engraftment, as indicated by B2 m^{b} positivity, were then irradiated again and transplanted with 5×10⁶ T cell-depleted bone marrow cells with or without 10×10⁶ Cd4-Cre (WT) or Cd4-Cre Lrrc8a^{fl/fl} CD4⁺ T cells. Mice were weighed daily and the degree of systemic GVHD was measured with a previously published clinical scoring system (3). Mice with clinical scores >6 or a weight loss of >20% were euthanized per the Boston Children's Hospital Animal Care and Use Committee standards. For measurement of circulating IFN-γ levels, blood was collected by retro-orbital bleeding seven days after transplantation and quantified using the mouse IFN gamma ELISA Ready-SET-Go! kit. For histologic analysis, transplanted mice were sacrificed and fixed in Bouin's fixative for five days, followed by paraffin embedding and sectioning. Slides were then H&E-stained and coded then examined in a blinded fashion using a previously published semi-quantitative scoring system for intestinal acute GVHD (3). Images of GVHD target tissue were acquired using a Leica DM LB2 microscope at a magnification of X200 and a Leica DFC 280 camera. For the isolation and analysis of colonic lamina propria T cells, colons were harvested, flushed with PBS supplemented with 2% FCS, and minced. To remove epithelial cells, the intestinal pieces were incubated in HBSS supplemented with 10 mmol/′, EDTA. 10 mmol/L HEPES, 0.5% FCS, and 1.5 mmol/L dithioerythritol for 20 minutes at 37° C. for two times. Intestinal pieces were then digested in HBSS with Ca²⁺/Mg²⁺, 20% FCS, 100 U/mL, collagenase VIII (Sigma-Aldrich), and 5 μg/mL DNase (Sigma-Aldrich) for 60 minutes at 37° C. Lamina propria cells were purified with a 40% Percoll gradient (GE Healthcare, Fairfield, Conn.). Flow cytometric analysis was then performed as described above.

Treg suppressor assay. CD4⁺ T cells were purified using magnetic negative selection (130-104-454, Miltenyi Biotec), they were then further purified using magnetic CD25 microbeads (130-091-041, Miltenyi Biotec) to yield CD4⁺CD25⁻ T cells (Teff) and CD4⁺CD25⁺ T cells (T_{regs}). Teffs were stimulated with anti-CD3 mAb (1 μg/ml, 17A2, Biolegend) in the presence of T cell-depleted splenocytes pre-treated with 25 μg/ml mitomycin C (Sigma) for five days. Proliferation was measured by flow cytometry.

Statistics. All data are presented as mean±standard error. The two-sided Student's t test was used for single comparisons. Statistical significance for multiple comparisons was quantified as specified in the figure legends.

### REFERENCES

1. Sharpe A H. Mechanisms of costimulation. Immunol. Rev. 2009; 229(1):5-11.
2. Croft M, Bradley L M, Swain S L. Naive versus memory CD4 T cell response to antigen. Memory cells are less dependent on accessory cell costimulation and can respond to many antigen-presenting cell types including resting B cells. J Immunol 1994; 162(6):2675-85.
3. Kumar R et al. Increased Sensitivity of Antigen-Experienced T Cells through the Enrichment of Oligomeric T Cell Receptor Complexes. Immunity 2011; 35(3):375-387.
4. Jones S C, Murphy G F, Friedman T M, Korngold R. Importance of minor histocompatibility antigen expression by nonhematopoietic tissues in a CD4+ T cell-mediated graft-versus-host disease model. J. Clin. Invest. 2003; 112(12):1880-1886.
5. Koyama M et al. Recipient nonhematopoietic antigen-presenting cells are sufficient to induce lethal acute graft-versus-host disease. Nat. Med. 2012; 18(1):135-142.
6. Boots A M, Wimmers-Bertens A J, Rijnders A W. Antigen-presenting capacity of rheumatoid synovial fibroblasts. Immunology 1994; 82(2):268-274.
7. Saada J I et al. Subepithelial Myofibroblasts are Novel Nonprofessional APCs in the Human Colonic Mucosa. J. Immunol. 2006; 177(9):5968-5979.
8. Hutton A J et al. Human Lung Fibroblasts Present Bacterial Antigens to Autologous Lung Th Cells. J. Immunol. 2017; 198(1):110-118.
9. Kumar L et al. Leucine-rich repeat containing 8A (LRRC8A) is essential for T lymphocyte development and function. J. Exp. Med. 2014; 211(5):929-942.
10. Qiu Z et al. SWELL1, a Plasma Membrane Protein, Is an Essential Component of Volume-Regulated Anion Channel. Cell 2014; 157(2):447-458.
11. Syeda R et al. LRRC8 proteins form volume-regulated anion channels that sense ionic strength. Cell 2016; 164(3):499-511.
12. Voss F K et al. Identification of LRRC8 Heteromers as an Essential Component of the Volume-Regulated Anion Channel VRAC. Science 2014; 344(6184):634-638.
13. Jentsch T J, Lutter D, Planells-Cases R, Ullrich F, Voss F K. VRAC: molecular identification as LRRC8 heteromers with differential functions. Pflüg. Arch. 2016; 468(3):385-393.
14. Bao J et al. Deficient LRRC8A-dependent volume-regulated anion channel activity is associated with male infertility in mice. JCI Insight 2018; 3(16). doi:10.1172/jci.insight.99767
15. Stuhlmann T, Planells-Cases R, Jentsch T J. LRRC8/VRAC anion channels enhance β-cell glucose sensing and insulin secretion. Nat. Commun. 2018; 9(1):1974.
16. Formaggio F et al. LRRC8A is essential for swelling-activated chloride current and for regulatory volume decrease in astrocytes. FASEB J. 2019; 33(1):101-113.
17. Platt C D et al. Leucine-rich repeat containing 8A (LRRC8A)-dependent volume-regulated anion channel activity is dispensable for T-cell development and function. J. Allergy Clin. Immunol. 2017; 140(6): 1651-1659.e1.
18. Zhang Y et al. SWELL1 is a regulator of adipocyte size, insulin signalling and glucose homeostasis. Nat. Cell Biol. 2017; 19(5):504-517.
19. Lu J, Xu F, Zhang J. Inhibition of angiotensin II-induced cerebrovascular smooth muscle cell proliferation by LRRC8A downregulation through suppressing PI3K/AKT activation. Hum. Cell 2019; 32(3):316-325.
20. Yao Z, Petschnigg J, Ketteler R, Stagljar I. Application guide for omics approaches to cell signaling. Nat. Chem. Biol. 2015; 11(6):387-397.
21. Cheroutre H, Husain M M. CD4 CTL: Living up to the challenge. Semin. Immunol. 2013; 25(4):273-281.
22. Koyama M, Hill G R. Alloantigen presentation and graft-versus-host disease: fuel for the fire. Blood 2016; 127(24):2963-2970.
23. Li H et al. Profound Depletion of Host Conventional Dendritic Cells, Plasmacytoid Dendritic Cells, and B Cells Does Not Prevent Graft-versus-Host Disease Induction. J. Immunol. 2012; 188(8):3804-3811.
24. Li H et al. Langerhans cells are not required for graft-versus-host disease. Blood 2011; 117(2):697-707.
25. Ashouri J F, Weiss A. Endogenous Nur77 is a specific indicator of antigen receptor signaling in human T and B cells. J. Immunol. Baltim. Md 1950 2017; 198(2):657-668.
26. Bantug G R, Galluzzi L, Kroemer G, Hess C. The spectrum of T cell metabolism in health and disease. Nat. Rev. Immunol. 2018; 18(1):19-34.
27. Nicholas D et al. Advances in the quantification of mitochondrial function in primary human immune cells through extracellular flux analysis. PLoS ONE 2017; 12(2):e0170975.
28. Macintyre A N et al. The Glucose Transporter Glut1 Is Selectively Essential for CD4 T Cell Activation and Effector Function. Cell Metab. 2014; 20(1):61-72.
29. Akkaya B et al. Increased Mitochondrial Biogenesis and Reactive Oxygen Species Production Accompany Prolonged CD4+ T Cell Activation. J. Immunol. 2018; 201(11):3294-3306.
30. Mookerjee S A, Nicholls D G, Brand M D. Determining Maximum Glycolytic Capacity Using Extracellular Flux Measurements [Internet]. PLoS ONE 2016; 11(3). doi:10.1371/journal.pone.0152016
31. Petrovic A et al. LPAM (α4β7 integrin) is an important homing integrin on alloreactive T cells in the development of intestinal graft-versus-host disease. Blood 2004; 103(4):1542-1547.
32. Davidson D, Shi X, Zhong M-C, Rhee I, Veillette A. The Phosphatase PTP-PEST Promotes Secondary T Cell Responses by Dephosphorylating the Protein Tyrosine Kinase Pyk2. Immunity 2010; 33(2):167-180.

1) A method for treating or preventing graft versus host disease, the method comprising administering to a subject having, or at risk of developing, graft versus host disease an agent that inhibits LRRC8A.

2) The method of claim 1, further comprising, prior to administering, the step of diagnosing a subject as having, or at risk of developing, graft versus host disease, or
the step of receiving the results from an assay that identifies a subject as having, or at risk of developing, graft versus host disease.

3) (canceled)

4) The method of claim 1, wherein subject is an organ transplant or hematopoietic stem cell transplant recipient.

5) The method of claim 1, wherein LRRC8A is inhibited in a T cell or antigen presenting cell.

6) (canceled)

7) The method of claim 1, wherein the agent that inhibits LRRC8A is selected from the group consisting of a small molecule, an antibody, a peptide, a genome editing system, an antisense oligonucleotide, and an RNAi.

8) The method of claim 7, wherein the antibody targets an antigen having a sequence selected from the group consisting of: SEQ ID NO: 3 and SEQ ID NO: 4.

9) The method of claim 7, wherein the RNAi is a microRNA, an siRNA, or a shRNA.

10) The method of claim 1, wherein inhibiting LRRC8A is inhibiting the expression level and/or activity of LRRC8A.

11) The method of claim 10, wherein the expression level and/or activity of LRRC8A is inhibited by at least 50%, at least 60%, at least 70%, at least 80%, at least 90%, or more as compared to an appropriate control.

12) The method of claim 1, further comprising administering at least a second therapeutic.

13) The method of claim 12, wherein the second therapeutic is Abatacept (Orencia®) or Belatacept (Nulojix®).

14)-19) (canceled)

20) A method for treating diabetes, the method comprising administering to a subject in need thereof an agent that inhibits LRRC8A.

21) The method of claim 20, further comprising, prior to administering, the step of diagnosing a subject as having diabetes.

22) The method of claim 20, further comprising, prior to administering, the step of receiving the results from an assay that identifies a subject as having diabetes.

23)-37) (canceled)

38) A composition comprising an agent that inhibits LRRC8A.

39) The composition of claim 38, further comprising at least a second therapeutic.

40) The composition of claim 38, wherein the second therapeutic is selected from the group consisting of: insulin, Abatacept (Orencia®) or Belatacept (Nulojix®).

41) The composition of any of claim 38, further comprising a pharmaceutically acceptable carrier or diluent.

42)-44) (canceled)

45) The composition of claim 38, wherein the agent that inhibits LRRC8A is selected from the group consisting of a small molecule, an antibody, a peptide, a genome editing system, an antisense oligonucleotide, and an RNAi.

46) The composition of claim 45, wherein the antibody binds an antigen having a sequence selected from the group consisting of: SEQ ID NO: 3 and SEQ ID NO: 4
